(19) United States
(12) Patent Application Publication
(10) Pub. No.: US 2006/0110803 A1
(43) Pub. Date: May 25, 2006

(54) Antigenic peptides of SARS coronavirus and uses thereof

(75) Inventors: Jan Henrik Ter Meulen, Amsterdam (NL); Jaap Goudsmit, Amsterdam (NL); Wouter Cornelis Puijk, Lelystad (NL); Jelle Wouter Slootstra, Lelystad (NL); Peter Timmerman, Lelystad (NL)

Correspondence Address: TRASK BRITT, P.O. BOX 2550, SALT LAKE CITY, UT, 84110, US

(73) Assignee: Crucell Holland B.V., Leiden (NL)

(21) Appl. No.: 11/295,192

(22) Filed: Dec. 6, 2005

Related U.S. Application Data
(63) Continuation of application No. PCT/EP04/51102, filed on Jun. 14, 2004.

(30) Foreign Application Priority Data
Jun. 13, 2003 (WO) PCT/EP03/50228
Jul. 28, 2003 (WO) PCT/EP03/50339
Sep. 3, 2003 (WO) PCT/EP03/50395
Oct. 27, 2003 (WO) PCT/EP03/50760
Nov. 17, 2003 (WO) PCT/EP03/50842

(51) Int. Cl. C12Q 1/68; C12P 19/34

(52) U.S. Cl. 435/91.1; 435/6

(57) ABSTRACT

The present invention pertains to antigenic peptides of SARS-CoV and their use in diagnostic test methods and in the treatment of condition resulting from SRAS-CoV. Furthermore, this invention provides antibodies capable of specifically recognizing the peptides of the invention. The antibodies can also advantageously be used in diagnostic test methods and in the treatment of condition resulting from SRAS-CoV.

### CROSS-REFERENCE TO RELATED APPLICATION

This application claims priority to International Application No. PCT/EP2004/051102, filed Jun. 14, 2004, published in English as PCT International Publication No. WO 2004/111081 A2 on Dec. 23, 2004, which application claims priority to International Application No. PCT/EP03/50228, filed Jun. 13, 2003, International Application No. PCT/EP03/50339, filed Jul. 28, 2003, International Application No. PCT/EP03/50395, filed Sep. 3, 2003, International Application No. PCT/EP03/50760, filed Oct. 27, 2003, and International Application No. PCT/EP03/50842, filed Nov. 17, 2003, the contents of each of which are incorporated by this reference.

### STATEMENT ACCORDING 37 C.F.R. § 1.52(e)(5)—SEQUENCE LISTING SUBMITTED ON COMPACT DISC

Pursuant to 37 C.F.R. § 1.52(e)(1)(iii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “2578-7530US-sequence listing.txt” which is 318 KB and created on Nov. 30, 2005.

### BACKGROUND OF THE INVENTION

### FIELD OF THE INVENTION

In general, embodiments of the present invention relate to biotechnology. The invention relates to medicine. In various embodiments, the invention relates to antigenic peptides of SARS coronavirus and uses thereof.

Recently a new and in several cases deadly clinical syndrome was observed in the human population, now called severe acute respiratory syndrome (SARS) (Holmes, 2003). The syndrome is caused by a novel coronavirus (Ksiazek et al., 2003), referred to as the SARS-CoV. The genome sequence of SARS-CoV has been determined (Rota et al., 2003; Marra et al., 2003). However, much remains to be learned about this virus, and means and methods for diagnostics, prevention and treatment of the virus and the syndrome are needed. The present invention provides means and methods for use in diagnostics, treatment and prevention of SARS-CoV.

### SUMMARY OF THE INVENTION

Embodiments of thehe present invention pertains to antigenic peptides of SARS-CoV. Furthermore, the invention provides fusion proteins comprising these peptides and antibodies against these peptides. The use of the peptides, fusion proteins and antibodies in the treatment of a condition resulting from SARS-CoV and a diagnostic test method for determining the presence of antibodies recognizing SARS-CoV in a sample or for determining the presence of SARS-CoV in a sample are also contemplated in the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: PEPSCAN-analysis of the spike-protein from SARS-CoV. The dark peaks show the binding of antibodies in the serum of a patient infected with SARS-CoV. The light peaks show the binding of antibodies in the serum of a patient recovered from SARS. Binding is tested in a PEPSCAN-based enzyme-linked immuno assay and quantified with a CCD-camera and an image processing system.

FIG. 2: Amino acid sequence of the spike protein from SARS-CoV (strain Urbani).

FIG. 3: Alignment of the spike glycoproteins of human enteric coronavirus OC43 and SARS coronavirus strain Urbani by means of the alignment program CLUSTAL W (“*” indicates identity between amino acids; “:” indicates chemically highly conserved amino acids; “.” indicates chemically less conserved amino acids). The boxed peptides indicated in the SARS-CoV spike glycoprotein are peptides that are recognized by sera of SARS-patients and by sera of control individuals and that have a high homology with corresponding peptides in the spike glycoprotein of OC43.

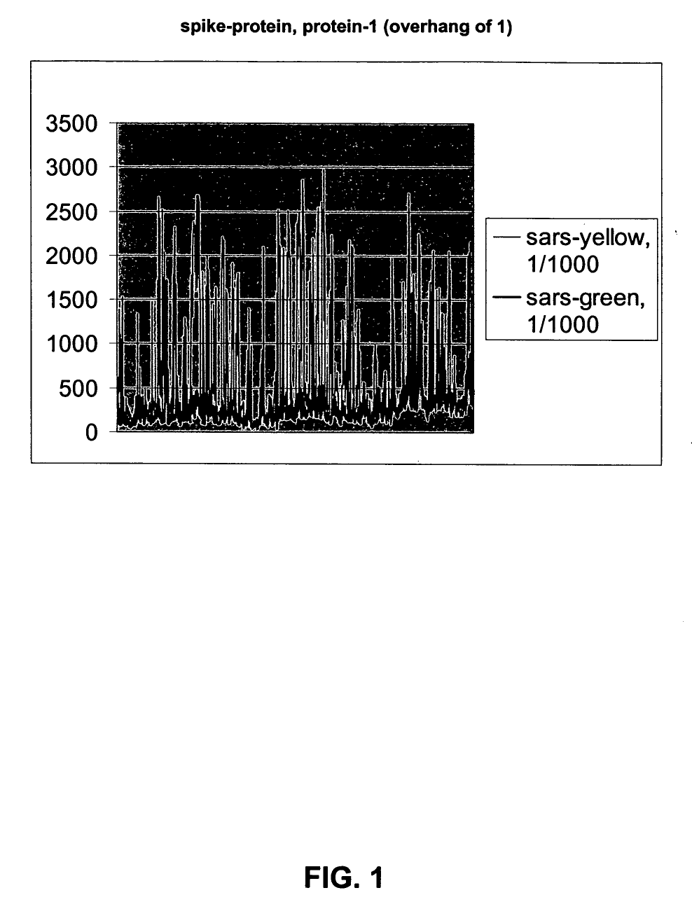

### DETAILED DESCRIPTION OF THE INVENTION

Several SARS-CoV strains including, but not limited to, the strains Urbani, TOR2, Frankfurt 1 and HSR 1, have been identified. The complete genome of these strains can be found in the EMBL-database and/or other databases. The genome of the strain called Urbani can be found under EMBL-database accession number AY278741. The coding sequence (CDS) of the (potential) proteins of SARS-CoV Urbani is also shown under EMBL-database accession number AY278741. The accession number in the EMBL-database of the complete genome of the strains TOR2, Frankfurt 1 and HSR 1 is AY274119, AY291315 and AY323977, respectively. Under these accession numbers the amino acid sequence of (potential) proteins of these strains can also be found.

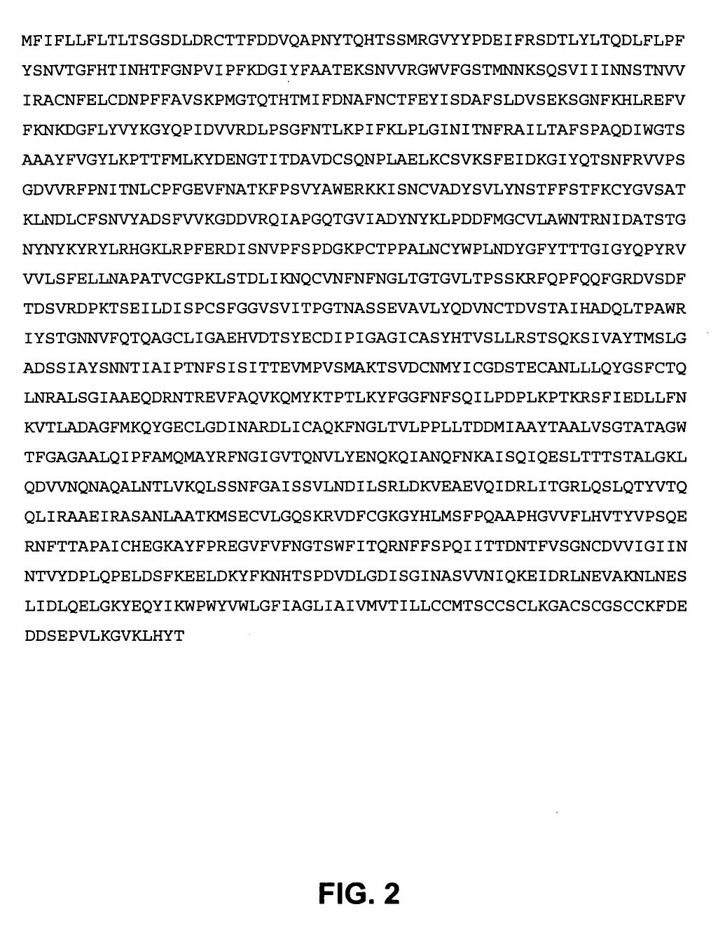

In a first aspect, the invention provides antigenic peptides of SARS-CoV. In the present invention, binding of sera from SARS patients to a series of overlapping 15-mer peptides, which were either in linear form or in looped/cyclic form, of the spike protein from SARS-CoV, in particular the SARS-CoV strain Urbani, was analyzed by means of PEPSCAN analysis (see inter alia WO 84/03564, WO 93/09872, Slootstra et al. 1996). The spike protein of SARS strain Urbani (the protein-id of the surface spike glycoprotein of SARS-CoV Urbani in the EMBL-database is AAP13441; for the amino acid sequence of the spike protein of Urbani see also FIG. 2 and SEQ ID NO: 39) is identical or highly homologous to the spike protein in other SARS-CoV strains. The protein-id of the surface spike glycoprotein of for instance the SARS-CoV strains TOR2, Frankfurt 1 and HSR 1 in the EMBL-database is AAP41037, AAP33697 and AAP72986. The antigenic peptides found in the present invention may not only be used for detection of the SARS-CoV strain Urbani and the prevention and/or treatment of a condition resulting from the SARS-CoV strain Urbani, but may also be useful in detecting SARS-CoV in general and preventing and/or treating a condition resulting from SARS-CoV in general.

In one embodiment, the invention provides a peptide having an amino acid sequence selected from the group consisting of

| | |
|---|---|
| MFIFLLFLTLTSGSD, | (SEQ ID NO:40) |
| FIFLLFLTLTSGSDL, | (SEQ ID NO:41) |
| IFLLFLTLTSGSDLD, | (SEQ ID NO:42) |
| FLLFLTLTSGSDLDR, | (SEQ ID NO:43) |
| LLFLTLTSGSDLDRC, | (SEQ ID NO:44) |
| LFLTLTSGSDLDRCT, | (SEQ ID NO:45) |
| FLTLTSGSDLDRCTT, | (SEQ ID NO:46) |
| LTLTSGSDLDRCTTF, | (SEQ ID NO:47) |
| TLTSGSDLDRCTTFD, | (SEQ ID NO:48) |
| LTSGSDLDRCTTFDD, | (SEQ ID NO:49) |
| TSGSDLDRCTTFDDV, | (SEQ ID NO:50) |
| SGSDLDRCTTFDDVQ, | (SEQ ID NO:51) |
| GSDLDRCTTFDDVQA, | (SEQ ID NO:52) |
| TQHTSSMRGVYYPDE, | (SEQ ID NO:70) |
| QHTSSMRGVYYPDEI, | (SEQ ID NO:71) |
| HTSSMRGVYYPDEIF, | (SEQ ID NO:72) |
| TSSMRGVYYPDEIFR, | (SEQ ID NO:73) |
| SSMRGVYYPDEIFRS, | (SEQ ID NO:74) |
| SMRGVYYPDEIFRSD, | (SEQ ID NO:75) |
| MRGVYYPDEIFRSDT, | (SEQ ID NO:76) |
| RGVYYPDEIFRSDTL, | (SEQ ID NO:77) |
| GVYYPDEIFRSDTLY, | (SEQ ID NO:78) |
| VYYPDEIFRSDTLYL, | (SEQ ID NO:79) |
| TGFHTINHTFGNPVI, | (SEQ ID NO:106) |
| GFHTINHTFGNPVIP, | (SEQ ID NO:107) |
| FHTINHTFGNPVIPF, | (SEQ ID NO:108) |
| HTINHTFGNPVIPFK, | (SEQ ID NO:109) |
| TINHTFGNPVIPFKD, | (SEQ ID NO:110) |
| INHTFGNPVIPFKDG, | (SEQ ID NO:111) |
| NHTFGNPVIPFKDGI, | (SEQ ID NO:112) |
| VSKPMGTQTHTMIFD, | (SEQ ID NO:179) |
| SKPMGTQTHTMIFDN, | (SEQ ID NO:180) |
| KPMGTQTHTMIFDNA, | (SEQ ID NO:181) |
| PMGTQTHTMIFDNAF, | (SEQ ID NO:182) |
| MGTQTHTMIFDNAFN, | (SEQ ID NO:183) |
| GTQTHTMIFDNAFNC, | (SEQ ID NO:184) |
| TQTHTMIFDNAFNCT, | (SEQ ID NO:185) |
| QTHTMIFDNAFNCTF, | (SEQ ID NO:186) |
| THTMIFDNAFNCTFE, | (SEQ ID NO:187) |
| AFSLDVSEKSGNFKH, | (SEQ ID NO:2) |
| FSLDVSEKSGNFKHL, | (SEQ ID NO:3) |
| SLDVSEKSGNFKHLR, | (SEQ ID NO:4) |
| LDVSEKSGNFKHLRE, | (SEQ ID NO:5) |
| DVSEKSGNFKHLREF, | (SEQ ID NO:6) |
| VSEKSGNFKHLREFV, | (SEQ ID NO:7) |
| SEKSGNFKHLREFVF, | (SEQ ID NO:8) |
| EKSGNFKHLREFVFK, | (SEQ ID NO:9) |
| ENGTITDAVDCSQNP, | (SEQ ID NO:296) |
| NGTITDAVDCSQNPL, | (SEQ ID NO:297) |
| GTITDAVDCSQNPLA, | (SEQ ID NO:298) |
| TITDAVDCSQNPLAE, | (SEQ ID NO:299) |
| ITDAVDCSQNPLAEL, | (SEQ ID NO:300) |
| TDAVDCSQNPLAELK, | (SEQ ID NO:301) |
| DAVDCSQNPLAELKC, | (SEQ ID NO:302) |
| AVDCSQNPLAELKCS, | (SEQ ID NO:303) |
| VDCSQNPLAELKCSV, | (SEQ ID NO:304) |
| DCSQNPLAELKCSVK, | (SEQ ID NO:305) |
| CSQNPLAELKCSVKS, | (SEQ ID NO:306) |
| SQNPLAELKCSVKSF, | (SEQ ID NO:307) |
| QNPLAELKCSVKSFE, | (SEQ ID NO:308) |
| NPLAELKCSVKSFEI, | (SEQ ID NO:309) |
| PLAELKCSVKSFEID, | (SEQ ID NO:310) |
| LAELKCSVKSFEIDK, | (SEQ ID NO:311) |
| QTSNFRVVPSGDVVR, | (SEQ ID NO:329) |
| TSNFRVVPSGDVVRF, | (SEQ ID NO:330) |
| SNFRVVPSGDVVRFP, | (SEQ ID NO:331) |
| NFRVVPSGDVVRFPN, | (SEQ ID NO:332) |
| FRVVPSGDVVRFPNI, | (SEQ ID NO:333) |
| RVVPSGDVVRFPNIT, | (SEQ ID NO:334) |
| VVPSGDVVRFPNITN, | (SEQ ID NO:335) |
| VPSGDVVRFPNITNL, | (SEQ ID NO:336) |
| PSGDVVRFPNITNLC, | (SEQ ID NO:337) |
| SGDVVRFPNITNLCP, | (SEQ ID NO:338) |
| GDVVRFPNITNLCPF, | (SEQ ID NO:339) |
| DVVRFPNITNLCPFG, | (SEQ ID NO:340) |
| VVRFPNITNLCPFGE, | (SEQ ID NO:341) |
| SNVPFSPDGKPCTPP, | (SEQ ID NO:484) |
| NVPFSPDGKPCTPPA, | (SEQ ID NO:485) |
| VPFSPDGKPCTPPAL, | (SEQ ID NO:486) |
| PFSPDGKPCTPPALN, | (SEQ ID NO:487) |
| FSPDGKPCTPPALNC, | (SEQ ID NO:488) |
| SPDGKPCTPPALNCY, | (SEQ ID NO:489) |
| PCTPPALNCYWPLND, | (SEQ ID NO:494) |
| CTPPALNCYWPLNDY, | (SEQ ID NO:495) |
| TPPALNCYWPLNDYG, | (SEQ ID NO:496) |
| PPALNCYWPLNDYGF, | (SEQ ID NO:497) |
| SFELLNAPATVCGPK, | (SEQ ID NO:528) |
| FELLNAPATVCGPKL, | (SEQ ID NO:529) |
| ELLNAPATVCGPKLS, | (SEQ ID NO:530) |
| LLNAPATVCGPKLST, | (SEQ ID NO:531) |
| LNAPATVCGPKLSTD, | (SEQ ID NO:532) |
| NAPATVCGPKLSTDL, | (SEQ ID NO:533) |
| APATVCGPKLSTDLI, | (SEQ ID NO:534) |
| PATVCGPKLSTDLIK, | (SEQ ID NO:535) |
| ATVCGPKLSTDLIKN, | (SEQ ID NO:536) |
| TVCGPKLSTDLIKNQ, | (SEQ ID NO:537) |
| VCGPKLSTDLIKNQC, | (SEQ ID NO:538) |
| CGPKLSTDLIKNQCV, | (SEQ ID NO:539) |
| GPKLSTDLIKNQCVN, | (SEQ ID NO:540) |
| PKLSTDLIKNQCVNF, | (SEQ ID NO:541) |
| KLSTDLIKNQCVNFN, | (SEQ ID NO:542) |
| SFGGVSVITPGTNAS, | (SEQ ID NO:605) |
| FGGVSVITPGTNASS, | (SEQ ID NO:606) |
| GGVSVITPGTNASSE, | (SEQ ID NO:607) |
| GVSVITPGTNASSEV, | (SEQ ID NO:608) |
| VSVITPGTNASSEVA, | (SEQ ID NO:609) |
| SVITPGTNASSEVAV, | (SEQ ID NO:610) |
| VITPGTNASSEVAVL, | (SEQ ID NO:611) |
| ITPGTNASSEVAVLY, | (SEQ ID NO:612) |
| TPGTNASSEVAVLYQ, | (SEQ ID NO:613) |
| PGTNASSEVAVLYQD, | (SEQ ID NO:614) |
| GTNASSEVAVLYQDV, | (SEQ ID NO:615) |
| TNASSEVAVLYQDVN, | (SEQ ID NO:616) |
| QAGCLIGAEHVDTSY, | (SEQ ID NO:660) |
| AGCLIGAEHVDTSYE, | (SEQ ID NO:661) |
| GCLIGAEHVDTSYEC, | (SEQ ID NO:662) |
| CLIGAEHVDTSYECD, | (SEQ ID NO:663) |
| LIGAEHVDTSYECDI, | (SEQ ID NO:664) |
| IGAEHVDTSYECDIP, | (SEQ ID NO:665) |
| GAEHVDTSYECDIPI, | (SEQ ID NO:666) |
| AEHVDTSYECDIPIG, | (SEQ ID NO:667) |
| EHVDTSYECDIPIGA, | (SEQ ID NO:668) |
| HVDTSYECDIPIGAG, | (SEQ ID NO:669) |
| VDTSYECDIPIGAGI, | (SEQ ID NO:670) |
| ITTEVMPVSMAKTSV, | (SEQ ID NO:732) |
| TTEVMPVSMAKTSVD, | (SEQ ID NO:733) |
| TEVMPVSMAKTSVDC, | (SEQ ID NO:734) |
| EVMPVSMAKTSVDCN, | (SEQ ID NO:735) |
| AKTSVDCNMYICGDS, | (SEQ ID NO:742) |
| KTSVDCNMYICGDST, | (SEQ ID NO:743) |
| TSVDCNMYICGDSTE, | (SEQ ID NO:744) |
| SVDCNMYICGDSTEC, | (SEQ ID NO:745) |
| VDCNMYICGDSTECA, | (SEQ ID NO:746) |
| DCNMYICGDSTECAN, | (SEQ ID NO:747) |
| CNMYICGDSTECANL, | (SEQ ID NO:748) |
| NMYICGDSTECANLL, | (SEQ ID NO:749) |
| MYICGDSTECANLLL, | (SEQ ID NO:750) |
| YICGDSTECANLLLQ, | (SEQ ID NO:751) |
| ICGDSTECANLLLQY, | (SEQ ID NO:752) |
| FNFSQILPDPLKPTK, | (SEQ ID NO:810) |
| NFSQILPDPLKPTKR, | (SEQ ID NO:811) |
| FSQILPDPLKPTKRS, | (SEQ ID NO:812) |
| SQILPDPLKPTKRSF, | (SEQ ID NO:813) |
| QILPDPLKPTKRSFI, | (SEQ ID NO:814) |
| ILPDPLKPTKRSFIE, | (SEQ ID NO:815) |
| LPDPLKPTKRSFIED, | (SEQ ID NO:816) |
| PDPLKPTKRSFIEDL, | (SEQ ID NO:817) |
| DPLKPTKRSFIEDLL, | (SEQ ID NO:818) |
| PLKPTKRSFIEDLLF, | (SEQ ID NO:819) |
| LKPTKRSFIEDLLFN, | (SEQ ID NO:820) |
| KPTKRSFIEDLLFNK, | (SEQ ID NO:821) |
| PTKRSFIEDLLFNKV, | (SEQ ID NO:822) |
| TKRSFIEDLLFNKVT, | (SEQ ID NO:823) |
| KRSFIEDLLFNKVTL, | (SEQ ID NO:824) |
| RSFIEDLLFNKVTLA, | (SEQ ID NO:825) |
| SFIEDLLFNKVTLAD, | (SEQ ID NO:826) |
| FIEDLLFNKVTLADA, | (SEQ ID NO:827) |
| IEDLLFNKVTLADAG, | (SEQ ID NO:828) |
| EDLLFNKVTLADAGF, | (SEQ ID NO:829) |
| DLLFNKVTLADAGFM, | (SEQ ID NO:830) |
| NKVTLADAGFMKQYG, | (SEQ ID NO:834) |
| KVTLADAGFMKQYGE, | (SEQ ID NO:835) |
| VTLADAGFMKQYGEC, | (SEQ ID NO:836) |
| TLADAGFMKQYGECL, | (SEQ ID NO:837) |
| LADAGFMKQYGECLG, | (SEQ ID NO:838) |
| ADAGFMKQYGECLGD, | (SEQ ID NO:839) |
| DAGFMKQYGECLGDI, | (SEQ ID NO:840) |
| AGFMKQYGECLGDIN, | (SEQ ID NO:841) |
| GFMKQYGECLGDINA, | (SEQ ID NO:842) |
| FMKQYGECLGDINAR, | (SEQ ID NO:843) |
| MKQYGECLGDINARD, | (SEQ ID NO:844) |
| KQYGECLGDINARDL, | (SEQ ID NO:845) |
| QYGECLGDINARDLI, | (SEQ ID NO:846) |
| YGECLGDINARDLIC, | (SEQ ID NO:847) |
| GECLGDINARDLICA, | (SEQ ID NO:848) |
| QKFNGLTVLPPLLTD, | (SEQ ID NO:863) |
| KFNGLTVLPPLLTDD, | (SEQ ID NO:864) |
| FNGLTVLPPLLTDDM, | (SEQ ID NO:865) |
| NGLTVLPPLLTDDMI, | (SEQ ID NO:866) |
| ALVSGTATAGWTFGA, | (SEQ ID NO:886) |
| LVSGTATAGWTFGAG, | (SEQ ID NO:887) |
| VSGTATAGWTFGAGA, | (SEQ ID NO:888) |
| SGTATAGWTFGAGAA, | (SEQ ID NO:889) |
| GTATAGWTFGAGAAL, | (SEQ ID NO:890) |
| TATAGWTFGAGAALQ, | (SEQ ID NO:891) |
| ATAGWTFGAGAALQI, | (SEQ ID NO:892) |
| TAGWTFGAGAALQIP, | (SEQ ID NO:893) |
| IGVTQNVLYENQKQI, | (SEQ ID NO:919) |
| GVTQNVLYENQKQIA, | (SEQ ID NO:920) |
| VTQNVLYENQKQIAN, | (SEQ ID NO:921) |
| TQNVLYENQKQIANQ, | (SEQ ID NO:922) |
| QNVLYENQKQIANQF, | (SEQ ID NO:923) |
| NVLYENQKQIANQFN, | (SEQ ID NO:924) |
| VLYENQKQIANQFNK, | (SEQ ID NO:925) |
| LYENQKQIANQFNKA, | (SEQ ID NO:926) |
| YENQKQIANQFNKAI, | (SEQ ID NO:927) |
| ENQKQIANQFNKAIS, | (SEQ ID NO:928) |
| NQKQIANQFNKAISQ, | (SEQ ID NO:929) |
| QKQIANQFNKAISQI, | (SEQ ID NO:930) |
| KQIANQFNKAISQIQ, | (SEQ ID NO:931) |
| QIQESLTTTSTALGK, | (SEQ ID NO:943) |
| IQESLTTTSTALGKL, | (SEQ ID NO:944) |
| QESLTTTSTALGKLQ, | (SEQ ID NO:945) |
| ESLTTTSTALGKLQD, | (SEQ ID NO:946) |
| SLTTTSTALGKLQDV, | (SEQ ID NO:947) |
| LTTTSTALGKLQDVV, | (SEQ ID NO:948) |
| TTTSTALGKLQDVVN, | (SEQ ID NO:949) |
| TTSTALGKLQDVVNQ, | (SEQ ID NO:950) |
| ALGKLQDVVNQNAQA, | (SEQ ID NO:954) |
| LGKLQDVVNQNAQAL, | (SEQ ID NO:955) |
| GKLQDVVNQNAQALN, | (SEQ ID NO:956) |
| KLQDVVNQNAQALNT, | (SEQ ID NO:957) |
| LQDVVNQNAQALNTL, | (SEQ ID NO:958) |
| QDVVNQNAQALNTLV, | (SEQ ID NO:959) |
| DVVNQNAQALNTLVK, | (SEQ ID NO:960) |
| VVNQNAQALNTLVKQ, | (SEQ ID NO:961) |
| VNQNAQALNTLVKQL, | (SEQ ID NO:962) |
| NQNAQALNTLVKQLS, | (SEQ ID NO:963) |
| QNAQALNTLVKQLSS, | (SEQ ID NO:964) |
| NAQALNTLVKQLSSN, | (SEQ ID NO:965) |
| AQALNTLVKQLSSNF, | (SEQ ID NO:966) |
| QALNTLVKQLSSNFG, | (SEQ ID NO:967) |
| ALNTLVKQLSSNFGA, | (SEQ ID NO:968) |
| SRLDKVEAEVQIDRL, | (SEQ ID NO:992) |
| RLDKVEAEVQIDRLI, | (SEQ ID NO:993) |
| LDKVEAEVQIDRLIT, | (SEQ ID NO:994) |
| DKVEAEVQIDRLITG, | (SEQ ID NO:995) |
| IRAAEIRASANLAAT, | (SEQ ID NO:1023) |
| RAAEIRASANLAATK, | (SEQ ID NO:14) |
| AAEIRASANLAATKM, | (SEQ ID NO:15) |
| AEIRASANLAATKMS, | (SEQ ID NO:16) |
| EIRASANLAATKMSE, | (SEQ ID NO:1024) |
| IRASANLAATKMSEC, | (SEQ ID NO:1025) |
| RASANLAATKMSECV, | (SEQ ID NO:1026) |
| ASANLAATKMSECVL, | (SEQ ID NO:1027) |
| SANLAATKMSECVLG, | (SEQ ID NO:1028) |
| ANLAATKMSECVLGQ, | (SEQ ID NO:1029) |
| NLAATKMSECVLGQS, | (SEQ ID NO:1030) |
| LAATKMSECVLGQSK, | (SEQ ID NO:1031) |
| GYHLMSFPQAAPHGV, | (SEQ ID NO:1052) |
| YHLMSFPQAAPHGVV, | (SEQ ID NO:18) |
| HLMSFPQAAPHGVVF, | (SEQ ID NO:19) |
| LMSFPQAAPHGVVFL, | (SEQ ID NO:1053) |
| MSFPQAAPHGVVFLH, | (SEQ ID NO:1054) |
| SFPQAAPHGVVFLHV, | (SEQ ID NO:20) |
| FPQAAPHGVVFLHVT, | (SEQ ID NO:1055) |
| PQAAPHGVVFLHVTY, | (SEQ ID NO:1056) |
| VTYVPSQERNFTTAP, | (SEQ ID NO:1068) |
| TYVPSQERNFTTAPA, | (SEQ ID NO:21) |
| YVPSQERNFTTAPAI, | (SEQ ID NO:22) |
| VPSQERNFTTAPAIC, | (SEQ ID NO:23) |
| PSQERNFTTAPAICH, | (SEQ ID NO:1069) |
| SQERNFTTAPAICHE, | (SEQ ID NO:24) |
| QERNFTTAPAICHEG, | (SEQ ID NO:1070) |
| ERNFTTAPAICHEGK, | (SEQ ID NO:1071) |
| RNFTTAPAICHEGKA, | (SEQ ID NO:1072) |
| NFTTAPAICHEGKAY, | (SEQ ID NO:25) |
| FTTAPAICHEGKAYF, | (SEQ ID NO:1073) |
| TTAPAICHEGKAYFP, | (SEQ ID NO:1074) |
| TAPAICHEGKAYFPR, | (SEQ ID NO:1075) |
| APAICHEGKAYFPRE, | (SEQ ID NO:1076) |
| PAICHEGKAYFPREG, | (SEQ ID NO:1077) |
| IINNTVYDPLQPELD, | (SEQ ID NO:1130) |
| INNTVYDPLQPELDS, | (SEQ ID NO:1131) |
| NNTVYDPLQPELDSF, | (SEQ ID NO:1132) |
| NTVYDPLQPELDSFK, | (SEQ ID NO:1133) |
| TVYDPLQPELDSFKE, | (SEQ ID NO:1134) |
| VYDPLQPELDSFKEE, | (SEQ ID NO:1135) |
| YDPLQPELDSFKEEL, | (SEQ ID NO:1136) |
| DPLQPELDSFKEELD, | (SEQ ID NO:1137) |
| PLQPELDSFKEELDK, | (SEQ ID NO:1138) |
| LQPELDSFKEELDKY, | (SEQ ID NO:1139) |
| QPELDSFKEELDKYF, | (SEQ ID NO:1140) |
| PELDSFKEELDKYFK, | (SEQ ID NO:1141) |
| ELDSFKEELDKYFKN, | (SEQ ID NO:1142) |
| LDSFKEELDKYFKNH, | (SEQ ID NO:1143) |
| DSFKEELDKYFKNHT, | (SEQ ID NO:1144) |
| ELDKYFKNHTSPDVD, | (SEQ ID NO:1147) |
| LDKYFKNHTSPDVDL, | (SEQ ID NO:1148) |
| DKYFKNHTSPDVDLG, | (SEQ ID NO:1149) |
| KYFKNHTSPDVDLGD, | (SEQ ID NO:1150) |
| YFKNHTSPDVDLGDI, | (SEQ ID NO:1151) |
| FKNHTSPDVDLGDIS, | (SEQ ID NO:1152) |
| KNHTSPDVDLGDISG, | (SEQ ID NO:1153) |
| NHTSPDVDLGDISGI, | (SEQ ID NO:1154) |
| HTSPDVDLGDISGIN, | (SEQ ID NO:1155) |
| TSPDVDLGDISGINA, | (SEQ ID NO:1156) |
| SPDVDLGDISGINAS, | (SEQ ID NO:1157) |
| DRLNEVAKNLNESLI, | (SEQ ID NO:1180) |
| RLNEVAKNLNESLID, | (SEQ ID NO:1181) |
| LNEVAKNLNESLIDL, | (SEQ ID NO:1182) |
| NEVAKNLNESLIDLQ, | (SEQ ID NO:1183) |
| EVAKNLNESLIDLQE, | (SEQ ID NO:1184) |
| VAKNLNESLIDLQEL, | (SEQ ID NO:1185) |
| AKNLNESLIDLQELG, | (SEQ ID NO:1186) |
| KNLNESLIDLQELGK, | (SEQ ID NO:1187) |
| NLNESLIDLQELGKY, | (SEQ ID NO:1188) |
| WYVWLGFIAGLIAIV, | (SEQ ID NO:1210) |
| YVWLGFIAGLIAIVM, | (SEQ ID NO:1211) |
| VWLGFIAGLIAIVMV, | (SEQ ID NO:1212) |
| WLGFIAGLIAIVMVT, | (SEQ ID NO:1213) |
| LGFIAGLIAIVMVTI, | (SEQ ID NO:1214) |
| GFIAGLIAIVMVTIL, | (SEQ ID NO:1215) |
| FIAGLIAIVMVTILL, | (SEQ ID NO:1216) |
| LCCMTSCCSCLKGAC, | (SEQ ID NO:1230) |
| CCMTSCCSCLKGACS, | (SEQ ID NO:1231) |
| CMTSCCSCLKGACSC, | (SEQ ID NO:1232) |
| MTSCCSCLKGACSCG, | (SEQ ID NO:1233) |
| TSCCSCLKGACSCGS, | (SEQ ID NO:1234) |
| SCCSCLKGACSCGSC, | (SEQ ID NO:26) |
| CCSCLKGACSCGSCC, | (SEQ ID NO:27) |
| CSCLKGACSCGSCCK, | (SEQ ID NO:28) |
| SCLKGACSCGSCCKF, | (SEQ ID NO:29) |
| CLKGACSCGSCCKFD, | (SEQ ID NO:30) |
| LKGACSCGSCCKFDE, | (SEQ ID NO:31) |
| KGACSCGSCCKFDED, | (SEQ ID NO:32) |
| GACSCGSCCKFDEDD, | (SEQ ID NO:33) |
| ACSCGSCCKFDEDDS, | (SEQ ID NO:34) |
| CSCGSCCKFDEDDSE, | (SEQ ID NO:35) |
| SCGSCCKFDEDDSEP, | (SEQ ID NO:36) |
| and | |
| CGSCCKFDEDDSEPV. | (SEQ ID NO:37) |

The peptides above are recognized in linear and/or looped/cyclic form by at least one of the following sera: a serum derived from an individual which has been infected by SARS-CoV and has recovered from SARS (the serum being called SARS-green); a serum derived from an individual in which the virus was still detectable by PCR and who suffered a prolonged and severe form of the illness (the serum being called SARS-yellow); sera form individuals which have been and/or are infected by SARS-CoV (the sera being called 1a (serum of 1 taken early in the course of the SARS-CoV infection), 1b (serum of 1 taken late in the course of the SARS-CoV infection), 2, 6, 37, 62 and London). It is clear for a person skilled in the art that the term “individuals which have been infected by SARS-CoV” as used herein also encompasses individuals which have been infected by SARS-CoV and are recovered from SARS.

Of the group of peptides presented above, the peptides having an amino acid sequence selected from the group consisting of SNVPFSPDGKPCTPP (SEQ ID NO:484),

| | |
|---|---|
| SNVPFSPDGKPCTPP, | (SEQ ID NO:484) |
| NVPFSPDGKPCTPPA, | (SEQ ID NO:485) |
| VPFSPDGKPCTPPAL, | (SEQ ID NO:486) |
| PFSPDGKPCTPPALN, | (SEQ ID NO:487) |
| FSPDGKPCTPPALNC, | (SEQ ID NO:488) |
| SPDGKPCTPPALNCY, | (SEQ ID NO:489) |
| AKTSVDCNMYICGDS, | (SEQ ID NO:742) |
| KTSVDCNMYICGDST, | (SEQ ID NO:743) |
| TSVDCNMYICGDSTE, | (SEQ ID NO:744) |
| SVDCNMYICGDSTEC, | (SEQ ID NO:745) |
| VDCNMYICGDSTECA, | (SEQ ID NO:746) |
| DCNMYICGDSTECAN, | (SEQ ID NO:747) |
| CNMYICGDSTECANL, | (SEQ ID NO:748) |
| NMYICGDSTECANLL, | (SEQ ID NO:749) |
| MYICGDSTECANLLL, | (SEQ ID NO:750) |
| YICGDSTECANLLLQ, | (SEQ ID NO:751) |
| ICGDSTECANLLLQY, | (SEQ ID NO:752) |
| ALVSGTATAGWTFGA, | (SEQ ID NO:886) |
| LVSGTATAGWTFGAG, | (SEQ ID NO:887) |
| VSGTATAGWTFGAGA, | (SEQ ID NO:888) |
| SGTATAGWTFGAGAA, | (SEQ ID NO:889) |
| GTATAGWTFGAGAAL, | (SEQ ID NO:890) |
| TATAGWTFGAGAALQ, | (SEQ ID NO:891) |
| ATAGWTFGAGAALQI, | (SEQ ID NO:892) |
| and | |
| TAGWTFGAGAALQIP | (SEQ ID NO:893) |

are recognized in linear form.

The peptides having an amino acid sequence selected from the group consisting of

| | |
|---|---|
| MFIFLLFLTLTSGSD, | (SEQ ID NO:40) |
| FIFLLFLTLTSGSDL, | (SEQ ID NO:41) |
| IFLLFLTLTSGSDLD, | (SEQ ID NO:42) |
| FLLFLTLTSGSDLDR, | (SEQ ID NO:43) |
| LLFLTLTSGSDLDRC, | (SEQ ID NO:44) |
| LFLTLTSGSDLDRCT, | (SEQ ID NO:45) |
| FLTLTSGSDLDRCTT, | (SEQ ID NO:46) |
| LTLTSGSDLDRCTTF, | (SEQ ID NO:47) |
| TLTSGSDLDRCTTFD, | (SEQ ID NO:48) |
| LTSGSDLDRCTTFDD, | (SEQ ID NO:49) |
| TSGSDLDRCTTFDDV, | (SEQ ID NO:50) |
| SGSDLDRCTTFDDVQ, | (SEQ ID NO:51) |
| GSDLDRCTTFDDVQA, | (SEQ ID NO:52) |
| TQHTSSMRGVYYPDE, | (SEQ ID NO:70) |
| QHTSSMRGVYYPDEI, | (SEQ ID NO:71) |
| HTSSMRGVYYPDEIF, | (SEQ ID NO:72) |
| TSSMRGVYYPDEIFR, | (SEQ ID NO:73) |
| SSMRGVYYPDEIFRS, | (SEQ ID NO:74) |
| SMRGVYYPDEIFRSD, | (SEQ ID NO:75) |
| MRGVYYPDEIFRSDT, | (SEQ ID NO:76) |
| RGVYYPDEIFRSDTL, | (SEQ ID NO:77) |
| GVYYPDEIFRSDTLY, | (SEQ ID NO:78) |
| VYYPDEIFRSDTLYL, | (SEQ ID NO:79) |
| PCTPPALNCYWPLND, | (SEQ ID NO:494) |
| CTPPALNCYWPLNDY, | (SEQ ID NO:495) |
| TPPALNCYWPLNDYG, | (SEQ ID NO:496) |
| PPALNCYWPLNDYGF, | (SEQ ID NO:497) |
| ITTEVMPVSMAKTSV, | (SEQ ID NO:732) |
| TTEVMPVSMAKTSVD, | (SEQ ID NO:733) |
| TEVMPVSMAKTSVDC, | (SEQ ID NO:734) |
| EVMPVSMAKTSVDCN, | (SEQ ID NO:735) |
| QKFNGLTVLPPLLTD, | (SEQ ID NO:863) |
| KFNGLTVLPPLLTDD, | (SEQ ID NO:864) |
| FNGLTVLPPLLTDDM, | (SEQ ID NO:865) |
| NGLTVLPPLLTDDMI, | (SEQ ID NO:866) |
| QIQESLTTTSTALGK, | (SEQ ID NO:943) |
| IQESLTTTSTALGKL, | (SEQ ID NO:944) |
| QESLTTTSTALGKLQ, | (SEQ ID NO:945) |
| ESLTTTSTALGKLQD, | (SEQ ID NO:946) |
| SLTTTSTALGKLQDV, | (SEQ ID NO:947) |
| LTTTSTALGKLQDVV, | (SEQ ID NO:948) |
| TTTSTALGKLQDVVN, | (SEQ ID NO:949) |
| TTSTALGKLQDVVNQ, | (SEQ ID NO:950) |
| ALGKLQDVVNQNAQA, | (SEQ ID NO:954) |
| LGKLQDVVNQNAQAL, | (SEQ ID NO:955) |
| GKLQDVVNQNAQALN, | (SEQ ID NO:956) |
| KLQDVVNQNAQALNT, | (SEQ ID NO:957) |
| LQDVVNQNAQALNTL, | (SEQ ID NO:958) |
| QDVVNQNAQALNTLV, | (SEQ ID NO:959) |
| DVVNQNAQALNTLVK, | (SEQ ID NO:960) |
| VVNQNAQALNTLVKQ, | (SEQ ID NO:961) |
| VNQNAQALNTLVKQL, | (SEQ ID NO:962) |
| NQNAQALNTLVKQLS, | (SEQ ID NO:963) |
| QNAQALNTLVKQLSS, | (SEQ ID NO:964) |
| NAQALNTLVKQLSSN, | (SEQ ID NO:965) |
| AQALNTLVKQLSSNF, | (SEQ ID NO:966) |
| QALNTLVKQLSSNFG, | (SEQ ID NO:967) |
| ALNTLVKQLSSNFGA, | (SEQ ID NO:968) |
| SRLDKVEAEVQIDRL, | (SEQ ID NO:992) |
| RLDKVEAEVQIDRLI, | (SEQ ID NO:993) |
| LDKVEAEVQIDRLIT, | (SEQ ID NO:994) |
| DKVEAEVQIDRLITG, | (SEQ ID NO:995) |
| IINNTVYDPLQPELD, | (SEQ ID NO:1130) |
| INNTVYDPLQPELDS, | (SEQ ID NO:1131) |
| NNTVYDPLQPELDSF, | (SEQ ID NO:1132) |
| NTVYDPLQPELDSFK, | (SEQ ID NO:1133) |
| TVYDPLQPELDSFKE, | (SEQ ID NO:1134) |
| VYDPLQPELDSFKEE, | (SEQ ID NO:1135) |
| YDPLQPELDSFKEEL, | (SEQ ID NO:1136) |
| DPLQPELDSFKEELD, | (SEQ ID NO:1137) |
| PLQPELDSFKEELDK, | (SEQ ID NO:1138) |
| LQPELDSFKEELDKY, | (SEQ ID NO:1139) |
| QPELDSFKEELDKYF, | (SEQ ID NO:1140) |
| PELDSFKEELDKYFK, | (SEQ ID NO:1141) |
| ELDSFKEELDKYFKN, | (SEQ ID NO:1142) |
| LDSFKEELDKYFKNH, | (SEQ ID NO:1143) |
| DSFKEELDKYFKNHT, | (SEQ ID NO:1144) |
| ELDKYFKNHTSPDVD, | (SEQ ID NO:1147) |
| LDKYFKNHTSPDVDL, | (SEQ ID NO:1148) |
| DKYFKNHTSPDVDLG, | (SEQ ID NO:1149) |
| KYFKNHTSPDVDLGD, | (SEQ ID NO:1150) |
| YFKNHTSPDVDLGDI, | (SEQ ID NO:1151) |
| FKNHTSPDVDLGDIS, | (SEQ ID NO:1152) |
| KNHTSPDVDLGDISG, | (SEQ ID NO:1153) |
| NHTSPDVDLGDISGI, | (SEQ ID NO:1154) |
| HTSPDVDLGDISGIN, | (SEQ ID NO:1155) |
| TSPDVDLGDISGINA, | (SEQ ID NO:1156) |
| SPDVDLGDISGINAS, | (SEQ ID NO:1157) |
| DRLNEVAKNLNESLI, | (SEQ ID NO:1180) |
| RLNEVAKNLNESLID, | (SEQ ID NO:1181) |
| LNEVAKNLNESLIDL, | (SEQ ID NO:1182) |
| NEVAKNLNESLIDLQ, | (SEQ ID NO:1183) |
| EVAKNLNESLIDLQE, | (SEQ ID NO:1184) |
| VAKNLNESLIDLQEL, | (SEQ ID NO:1185) |
| AKNLNESLIDLQELG, | (SEQ ID NO:1186) |
| KNLNESLIDLQELGK, | (SEQ ID NO:1187) |
| NLNESLIDLQELGKY, | (SEQ ID NO:1188) |
| WYVWLGFIAGLIAIV, | (SEQ ID NO:1210) |
| YVWLGFIAGLIAIVM, | (SEQ ID NO:1211) |
| VWLGFIAGLIAIVMV, | (SEQ ID NO:1212) |
| WLGFIAGLIAIVMVT, | (SEQ ID NO:1213) |
| LGFIAGLIAIVMVTI, | (SEQ ID NO:1214) |
| GFIAGLIAIVMVTIL, | (SEQ ID NO:1215) |
| and | |
| FIAGLIAIVMVTILL | (SEQ ID NO:1216) |

are recognized in looped/cyclic form.

All other peptides mentioned above are recognized in linear as well as looped/cyclic form.

Further embodiments of peptides comprise a peptide consisting of an amino acid sequence selected from the group consisting of:

| | |
|---|---|
| GTQTHTMIFDNAFNCT, | (SEQ ID NO:1248) |
| SLDVSEKSGNFKHLRE, | (SEQ ID NO:1249) |
| ENGTITDAVDCSQNPLAEL, | (SEQ ID NO:1250) |
| DAVDCSQNPLAELKCSVKSFEIDK, | (SEQ ID NO:1251) |
| SNVPFSPDGKPCTPPALNCY, | (SEQ ID NO:1252) |
| SFGGVSVITPGTNASSEVA, | (SEQ ID NO:1253) |
| QAGCLIGAEHVDTSYECDIP, | (SEQ ID NO:1254) |
| AKTSVDCNMYICGDSTECANL, | (SEQ ID NO:1255) |
| MYICGDSTECANLLLQY, | (SEQ ID NO:1256) |
| LKPTKRSFIEDLLFNKVTLA, | (SEQ ID NO:1257) |
| SCCSCLKGACSCGSCCK, | (SEQ ID NO:1258) |
| CLKGACSCGSCCKFDE, | (SEQ ID NO:1259) |
| TLTSGSDLDRCTTFDD, | (SEQ ID NO:1260) |
| FKNHTSPDVDLGDISG, | (SEQ ID NO:1261) |
| RLNEVAKNLNESLIDL, | (SEQ ID NO:1262) |
| VAKNLNESLIDLQELG, | (SEQ ID NO:1263) |
| and | |
| TSCCSCLKGACSCGSCC. | (SEQ ID NO:1264) |

The combined observations lead us to believe that the oligopeptides identified above are good candidates to represent epitopes of the SARS-CoV. The peptides of the invention may be advantageously used in diagnostic test methods as described herein. They may also be used in therapy and/or prevention of conditions resulting from an infection with SARS-CoV as described herein.

In a further aspect of the invention, peptides mentioned above may be coupled/linked to each other. Peptides of the embodiments of the invention may be linked/coupled to peptides of other embodiments of the invention or the same embodiment of the invention. The peptides may be linear and/or looped/cyclic. A combination peptide obtained this way may mimic/simulate a discontinuous and/or conformational epitope that is more antigenic than the single peptides. The combination peptide may also constitute more than two peptides. The peptides of the invention can be linked directly or indirectly via for instance a spacer of variable length. Furthermore, the peptides can be linked covalently or non-covalently. They may also be part of a fusion protein or conjugate. In general, the peptides should be in such a form as to be capable of mimicking/simulating a discontinuous and/or conformational epitope.

Obviously, the person skilled in the art may make modifications to the peptide without departing from the scope of the invention, e.g. by systematic length variation and/or replacement of residues and/or combination with other peptides. Peptides can be synthesized by known solid phase peptide synthesis techniques. The synthesis allows for one or more amino acids not corresponding to the original peptide sequence to be added to the amino or carboxyl terminus of the peptides. Such extra amino acids are useful for coupling the peptides to each other, to another peptide, to a large carrier protein or to a solid support. Amino acids that are inter alia useful for these purposes include tyrosine, lysine, glutamic acid, aspartic acid, cysteine and derivatives thereof. Additional protein modification techniques may be used, e.g., NH₂-acetylation or COOH-terminal amidation, to provide additional means for coupling the peptides to another protein or peptide molecule or to a support, for example, polystyrene or polyvinyl microtiter plates, glass tubes or glass beads or particles and chromatographic supports, such as paper, cellulose and cellulose derivates, and silica. If the peptide is coupled to such a support, it may also be used for affinity purification of SARS-CoV recognizing antibodies.

In an embodiment the peptides of the invention can have a looped/cyclic form. Linear peptides can be made by chemically converting the structures to looped/cyclic forms. It is well known in the art that cyclization of linear peptides can modulate bioactivity by increasing or decreasing the potency of binding to the target protein. Linear peptides are very flexible and tend to adopt many different conformations in solution. Cyclization acts to constrain the number of available conformations, and thus, favor the more active or inactive structures of the peptide. Cyclization of linear peptides is accomplished either by forming a peptide bond between the free N-terminal and C-terminal ends (homodetic cyclopeptides) or by forming a new covalent bond between amino acid backbone and/or side chain groups located near the N- or C-terminal ends (heterodetic cyclopeptides). The latter cyclizations use alternate chemical strategies to form covalent bonds, for example, disulfides, lactones, ethers, or thioethers. However, cyclization methods other than the ones described above can also be used to form cyclic/looped peptides. Generally, linear peptides of more than five residues can be cyclized relatively easily. The propensity of the peptide to form a beta-turn conformation in the central four residues facilitates the formation of both homo- and heterodetic cyclopeptides. The looped/cyclic peptides of the invention preferably comprise a cysteine residue at position 2 and 14. Preferably, they contain a linker between the cysteine residues. The looped/cyclic peptides of the invention are recognized by antibodies in the serum of individuals that have been and/or are infected with SARS-CoV.

Alternatively, the peptides of the invention may be prepared by expression of the peptides or of a larger peptide including the desired peptide from a corresponding gene (whether synthetic or natural in origin) in a suitable host. The larger peptide may contain a cleavage site whereby the peptide of interest may be released by cleavage of the fused molecule.

The resulting peptides may then be tested for binding to sera from subjects that have been previously infected with SARS-CoV, to sera form infected subjects or to purified SARS-CoV antibodies in a way essentially as described herein. If such a peptide can still be bound by the sera or antibody, it is considered as a functional fragment or analogue of the peptides according to the invention. Also, even stronger antigenic peptides may be identified in this manner, which peptides may be used for vaccination purposes or for generating strongly neutralizing antibodies for therapeutic and/or prophylactic purposes. The peptides may also be used in diagnostic tests. Therefore, the invention also provides the peptides comprising a part (or even consisting of a part) of a peptide according to the invention, wherein that part is recognized by antibodies present in serum derived from an individual that has been and/or is infected by SARS-CoV.

Furthermore, the invention provides peptides consisting of an analogue of a peptide according to the invention, wherein one or more amino acids are substituted for another amino acid, and wherein the analogue is recognized by antibodies present in serum derived from an individual that has been and/or is infected by SARS-CoV. Alternatively, analogues can be peptides of the present invention comprising an amino acid sequence containing insertions, deletions or combinations thereof of one or more amino acids compared to the amino acid sequences of the parent peptides. Furthermore, analogues can comprise truncations of the amino acid sequence at either or both the amino or carboxy termini of the peptides. Analogues according to the invention may have the same or different, either higher or lower, antigenic properties compared to the parent peptides, but are still recognized by antibodies present in serum derived from an individual that has been or is infected by SARS-CoV. That part of a 15-mer still representing immunogenic activity consists of about 6-12, preferably 8-10, more preferably nine amino acids within the 15-mer.

The peptides, parts thereof or analogues thereof according to the invention may be used directly as peptides, but may also be used conjugated to an immunogenic carrier, which may be, e.g., a polypeptide or polysaccharide. If the carrier is a polypeptide, the desired conjugate may be expressed as a fusion protein. Alternatively, the peptide and the carrier may be obtained separately and then conjugated. This conjugation may be covalently or non-covalently. A fusion protein is a chimeric protein, comprising the peptide according to the invention, and another protein or part thereof not being the SARS-CoV spike protein. Such fusion proteins may for instance be used to raise antibodies for diagnostic, prophylactic and/or therapeutic purposes or to directly immunize, i.e. vaccinate, humans or animals. Any protein or part thereof or even peptide may be used as fusion partner for the peptide according to the invention to form a fusion protein, and non-limiting examples are bovine serum albumin, keyhole limpet hemocyanin, etc.

The peptides may be labeled (signal-generating) or unlabeled. This depends on the type of assay used. Labels which may be coupled to the peptides are those known in the art and include, but are not limited to, enzymes, radionuclides, fluorogenic and chromogenic substrates, cofactors, biotin/avidin, colloidal gold, and magnetic particles.

It is another aspect of the invention to provide nucleic acid molecules encoding peptides, parts thereof or analogues thereof or fusion proteins according to the invention. Such nucleic acid molecules may suitably be used in the form of plasmids for propagation and expansion in bacterial or other hosts. Moreover, recombinant DNA techniques well known to the person skilled in the art can be used to obtain nucleic acid molecules encoding analogues of the peptides according to the invention, e.g. by mutagenesis of the sequences encoding the peptides according to the invention. The skilled man will appreciate that analogues of the nucleic acid molecules are also intended to be a part of the present invention. Analogues are nucleic acid sequences that can be directly translated, using the standard genetic code, to provide an amino acid sequence identical to that translated from the parent nucleic acid molecules. Another aspect of nucleic acid molecules according to the present invention, is their potential for use in gene-therapy or vaccination applications. Therefore, in another embodiment of the invention, nucleic acid molecules according to the invention are provided wherein the nucleic acid molecule is present in a gene delivery vehicle. A “gene delivery vehicle” as used herein refers to an entity that can be used to introduce nucleic acid molecules into cells, and includes liposomes, naked DNA, plasmid DNA, optionally coupled to a targeting moiety such as an antibody with specificity for an antigen presenting cell, recombinant viruses, and the like. Preferred gene therapy vehicles of the present invention will generally be viral vectors, such as comprised within a recombinant retrovirus, herpes simplex virus (HSV), adenovirus, adeno-associated virus (AAV), cytomegalovirus (CMV), and the like. Such applications of the nucleic acid sequences according to the invention are included in the present invention. The person skilled in the art will be aware of the possibilities of recombinant viruses for administering sequences of interest to cells. The administration of the nucleic acids of the invention to cells can result in an enhanced immune response. Alternatively, the nucleic acid encoding the peptides of the invention can be used as naked DNA vaccines, e.g. immunization by injection of purified nucleic acid molecules into humans or animals.

In another aspect, the invention provides antibodies recognizing the peptides, parts or analogues thereof of the invention. Antibodies can be obtained according to routine methods well known to the person skilled in the art, including but not limited to immunization of animals such as mice, rabbits, goats, and the like, or by antibody, phage or ribosome display methods (see e.g. Using Antibodies: A Laboratory Manual, Edited by: E. Harlow, D. Lane (1998), Cold Spring Harbor Laboratory Press, Cold Spring Harbor, N.Y.; Current Protocols in Immunology, Edited by: J. E. Coligan, A. M. Kruisbeek, D. H. Margulies, E. M. Shevach, W. Strober (2001), John Wiley & Sons Inc., New York; and Phage Display: A Laboratory Manual. Edited by: C. F. Barbas, D. R. Burton, J. K. Scott and G. J. Silverman (2001), Cold Spring Harbor Laboratory Press, Cold Spring Harbor, N.Y., the disclosures of which are incorporated herein by reference).

The antibodies of the invention can be intact immunoglobulin molecules such as polyclonal or monoclonal antibodies, in particular human monoclonal antibodies, or the antibodies can be functional fragments thereof, i.e. fragments that are still capable of binding to the antigen. These fragments include, but not limited to, Fab, F(ab′), F(ab′)₂, Fv, dAb, Fd, complementarity determining region (CDR) fragments, single-chain antibodies (scFv), bivalent single-chain antibodies, diabodies, triabodies, tetrabodies, and (poly)peptides that contain at least a fragment of an immunoglobulin that is sufficient to confer specific antigen binding to the (poly)peptides. The antibodies of the invention can be used in non-isolated or isolated form. Furthermore, the antibodies of the invention can be used alone or in a mixture/composition comprising at least one antibody (or variant or fragment thereof) of the invention. Antibodies of the invention include all the immunoglobulin classes and subclasses known in the art. Depending on the amino acid sequence of the constant domain of their heavy chains, binding molecules can be divided into the five major classes of intact antibodies: IgA, IgD, IgE, IgG, and IgM, and several of these may be further divided into subclasses (isotypes), e.g., IgA1, IgA2, IgG1, IgG2, IgG3 and IgG4. The above mentioned antigen-binding fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, Edited by: E. Harlow and D, Lane (1988), Cold Spring Harbor Laboratory, Cold Spring Harbor, N.Y., which is incorporated herein by reference. A binding molecule or antigen-binding fragment thereof may have one or more binding sites. If there is more than one binding site, the binding sites may be identical to one another or they may be different.

The antibodies of the invention can be naked or unconjugated antibodies. A naked or unconjugated antibody is intended to refer to an antibody that is not conjugated, operatively linked or otherwise physically or functionally associated with an effector moiety or tag, such as inter alia a toxic substance, a radioactive substance, a liposome, an enzyme. It will be understood that naked or unconjugated antibodies do not exclude antibodies that have been stabilized, multimerized, humanized or in any other way manipulated, other than by the attachment of an effector moiety or tag. Accordingly, all post-translationally modified naked and unconjugated antibodies are included herewith, including where the modifications are made in the natural antibody-producing cell environment, by a recombinant antibody-producing cell, and are introduced by the hand of man after initial antibody preparation. Of course, the term naked or unconjugated antibody does not exclude the ability of the antibody to form functional associations with effector cells and/or molecules after administration to the body, as some of such interactions are necessary in order to exert a biological effect. The lack of associated effector group or tag is therefore applied in definition to the naked or unconjugated binding molecule in vitro, not in vivo.

Alternatively, the antibodies as described in the present invention can be conjugated to tags and be used for detection and/or analytical and/or diagnostic purposes. The tags used to label the antibodies for those purposes depend on the specific detection/analysis/diagnosis techniques and/or methods used such as inter alia immunohistochemical staining of tissue samples, flow cytometric detection, scanning laser cytometric detection, fluorescent immunoassays, enzyme-linked immunosorbent assays (ELISAs), radioimmunoassays (RIAs), bioassays (e.g., neutralization assays, growth inhibition assays), Western blotting applications, etc. For immunohistochemical staining of tissue samples preferred labels are enzymes that catalyze production and local deposition of a detectable product. Enzymes typically conjugated to antibodies to permit their immunohistochemical visualization are well-known and include, but are not limited to, alkaline phosphatase, P-galactosidase, glucose oxidase, horseradish peroxidase, and urease. Typical substrates for production and deposition of visually detectable products include, but are not limited to, o-nitrophenyl-beta-D-galactopyranoside (ONPG), o-phenylenediamine dihydrochloride (OPD), p-nitrophenyl phosphate (PNPP), p-nitrophenyl-beta-D-galactopryanoside (PNPG), 3′, 3′-diaminobenzidine (DAB), 3-amino-9-ethylcarbazole (AEC), 4-chloro-1-naphthol (CN), 5-bromo-4-chloro-3-indolyl-phosphate (BCIP), ABTS, BluoGal, iodonitrotetrazolium (INT), nitroblue tetrazolium chloride (NBT), phenazine methosulfate (PMS), phenolphthalein monophosphate (PMP), tetramethyl benzidine (TMB), tetranitroblue tetrazolium (TNBT), X-Gal, X-Gluc, and X-glucoside. Other substrates that can be used to produce products for local deposition are luminescent substrates. For example, in the presence of hydrogen peroxide, horseradish peroxidase can catalyze the oxidation of cyclic diacylhydrazides such as luminol. Next to that, binding molecules of the immunoconjugate of the invention can also be labeled using colloidal gold or they can be labeled with radioisotopes, such as ³³p, ³²p, ³⁵S, ³H, and ¹²⁵I. When the antibodies of the present invention are used for flow cytometric detections, scanning laser cytometric detections, or fluorescent immunoassays, they can usefully be labeled with fluorophores. A wide variety of fluorophores useful for fluorescently labeling the antibodies of the present invention include, but are not limited to, Alexa Fluor and Alexa Fluor&commat dyes, BODIPY dyes, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Cy2, Cy3, Cy3.5, Cy5, Cy5.5, Cy7, fluorescein isothiocyanate (FITC), allophycocyanin (APC), R-phycoerythrin (PE), peridinin chlorophyll protein (PerCP), Texas Red, fluorescence resonance energy tandem fluorophores such as PerCP-Cy5.5, PE-Cy5, PE-Cy5.5, PE-Cy7, PE-Texas Red, and APC-Cy7. When the antibodies of the present invention are used for secondary detection using labeled avidin, streptavidin, captavidin or neutravidin, the antibodies may be labeled with biotin.

Next to that, the antibodies of the invention may be conjugated to photoactive agents or dyes such as fluorescent and other chromogens or dyes to use the so obtained immunoconjugates in photoradiation, phototherapy, or photodynamic therapy. The photoactive agents or dyes include, but are not limited to, photofrin.RTM, synthetic diporphyrins and dichlorins, phthalocyanines with or without metal substituents, chloroaluminum phthalocyanine with or without varying substituents, O-substituted tetraphenyl porphyrins, 3,1-meso tetrakis (o-propionamido phenyl) porphyrin, verdins, purpurins, tin and zinc derivatives of octaethylpurpurin, etiopurpurin, hydroporphyrins, bacteriochlorins of the tetra(hydroxyphenyl) porphyrin series, chlorins, chlorin e₆, mono-1-aspartyl derivative of chlorin e₆, di-1-aspartyl derivative of chlorin e₆, tin(IV) chlorin e₆, meta-tetrahydroxyphenylchlor- in, benzoporphyrin derivatives, benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin, sulfonated aluminum PC, sulfonated AlPc, disulfonated, tetrasulfonated derivative, sulfonated aluminum naphthalocyanines, naphthalocyanines with or without metal substituents and with or without varying substituents, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, naturally occurring porphyrins, hematoporphyrin, ALA-induced protoporphyrin IX, endogenous metabolic precursors, 5-aminolevulinic acid benzonaphthoporphyrazines, cationic imminium salts, tetracyclines, lutetium texaphyrin, tin-etio-purpurin, porphycenes, benzophenothiazinium and combinations thereof.

When the antibodies of the invention are used for in vivo diagnostic use, the antibodies can also be made detectable by conjugation to e.g. magnetic resonance imaging (MRI) contrast agents, such as gadolinium diethylenetriaminepentaacetic acid, to ultrasound contrast agents or to X-ray contrast agents, or by radioisotopic labeling.

The antibodies according to the invention may be capable of neutralizing SARS-CoV infectivity and are useful for therapeutic purposes against this virus. Assays to detect and measure virus neutralizing activity of antibodies are well known in the art. For example, a SARS-CoV neutralization assay can be performed on Vero cells (ATCC CCL 81). Antibodies of the invention are mixed with virus suspension and incubated for one hour at 37° C. The obtained suspension is then inoculated onto sub-confluent Vero cells (approx. 80% density) grown in 96-well cell-culture plates. The inoculated cells are cultured for 3-4 days at 37° C. and observed daily for the development of cytopathic effect (CPE). CPE is compared to the positive control (virus inoculated cells) and negative controls (mock-inoculated cells or cells incubated with antibody only). Alternatively, the antibodies may inhibit or down-regulate SARS-CoV replication, are complement fixing antibodies capable of assisting in the lysis of enveloped SARS-CoV and/or act as opsonins and augment phagocytosis of SARS-CoV either by promoting its uptake via Fc or C3b receptors or by agglutinating SARS-CoV to make it more easily phagocytosed.

The invention also provides nucleic acid molecules encoding the antibodies according to the invention.

It is another aspect of the invention to provide vectors, i.e. nucleic acid constructs, comprising one or more nucleic acid molecules according to the present invention. The nucleic acid molecule may either encode the peptides, parts or analogues thereof or fusion proteins of the invention or encode the antibodies of the invention. Vectors can be derived from plasmids such as inter alia F, R1, RP1, Co1, pBR322, TOL, Ti, etc; cosmids; phages such as lambda, lambdoid, M13, Mu, P1, P22, Q_{β}, T-even, T-odd, T2, T4, T7, etc; plant viruses such as inter alia alfalfa mosaic virus, bromovirus, capillovirus, carlavirus, carmovirus, caulivirus, clostervirus, comovirus, cryptovirus, cucumovirus, dianthovirus, fabavirus, fijivirus, furovirus, geminivirus, hordeivirus, ilarvirus, luteovirus, machlovirus, marafivirus, necrovirus, nepovirus, phytorepvirus, plant rhabdovirus, potexvirus, potyvirus, sobemovirus, tenuivirus, tobamovirus, tobravirus, tomato spotted wilt virus, tombusvirus, tymovirus, etc; or animal viruses such as inter alia adenovirus, arenaviridae, baculoviridae, birnaviridae, bunyaviridae, calciviridae, cardioviruses, coronaviridae, corticoviridae, cystoviridae, Epstein-Barr virus, enteroviruses, filoviridae, flaviviridae, Foot-and-Mouth disease virus, hepadnaviridae, hepatitis viruses, herpesviridae, immunodeficiency viruses, influenza virus, inoviridae, iridoviridae, orthomyxoviridae, papovaviruses, paramyxoviridae, parvoviridae, picornaviridae, poliovirus, polydnaviridae, poxviridae, reoviridae, retroviruses, rhabdoviridae, rhinoviruses, Semliki Forest virus, tetraviridae, togaviridae, toroviridae, vaccinia virus, vescular stomatitis virus, etc. Vectors can be used for cloning and/or for expression of the peptides, parts or analogues thereof of the invention or antibodies of the invention of the invention and might even be used for gene therapy purposes. Vectors comprising one or more nucleic acid molecules according to the invention operably linked to one or more expression-regulating nucleic acid molecules are also covered by the present invention. The choice of vector is dependent on the recombinant procedures followed and the host used. Introduction of vectors in host cells can be effected by inter alia calcium phosphate transfection, virus infection, DEAE-dextran mediated transfection, lipofectamin transfection or electroporation. Vectors may be autonomously replicating or may replicate together with the chromosome into which they have been integrated. Preferably, the vectors contain one or more selection markers. Useful markers are dependent on the host cells of choice and are well known to persons skilled in the art. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the peptides, parts or analogues thereof or antibodies as described above operably linked to one or more nucleic acid molecules encoding proteins or peptides that can be used to isolate these molecules are also covered by the invention. These proteins or peptides include, but are not limited to, glutathione-S-transferase, maltose binding protein, metal-binding polyhistidine, green fluorescent protein, luciferase and beta-galactosidase.

Hosts containing one or more copies of the vectors mentioned above are an additional subject of the present invention. Preferably, the hosts are cells. Preferably, the cells are suitably used for the manipulation and propagation of nucleic acid molecules. Suitable cells include, but are not limited to, cells of mammalian, plant, insect, fungal or bacterial origin. Bacterial cells include, but are not limited to, cells from Gram positive bacteria such as several species of the genera Bacillus, Streptomyces and Staphylococcus or cells of Gram negative bacteria such as several species of the genera Escherichia, such as Escherichia coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by known methods, for example, Agrobacterium-mediated gene transfer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells such as Chinese Hamster Ovary (CHO) cells, NS-0 cells, COS cells, BHK cells or Bowes melanoma cells are preferred in the present invention. Preferably, the cells are human retina cells that have been immortalized by adenovirus E1 sequences, such as PER.C6™ cells. PER.C6™ cells can be used for the expression of antibodies to high levels (see e.g. WO 00/63403) with human glycosylation patterns. The cells according to the invention may contain the nucleic acid molecule according to the invention in expressible format, such that the desired protein can be recombinantly expressed from the cells.

In a further aspect, the invention is directed to a peptide, part or analogue thereof according to the invention, preferably according to the first embodiment described above, or a fusion protein according to the invention or a nucleic acid molecule encoding a peptide, part or analogue thereof according to the invention or a nucleic acid molecule encoding a fusion protein of the invention for use as a medicament. In other words, the invention is directed to a method of prevention and/or treatment wherein a peptide, part or analogue thereof according to the invention, or a fusion protein according to the invention or a nucleic acid molecule encoding a peptide, part or analogue thereof according to the invention or a nucleic acid molecule encoding a fusion protein of the invention is used. Preferably, the peptides, parts or analogues thereof of the invention may for example be for use as an immunogen, preferably a vaccine.

If the peptides, parts and analogues thereof of the invention are in the form of a vaccine, they are preferably formulated into compositions. A composition may also comprise more than one peptide of the invention. These peptides may be different or identical and may be linked, covalently or non-covalently, to each other or not linked to each other. For formulation of such compositions, an immunogenically effective amount of at least one of the peptides of the invention is admixed with a physiologically acceptable carrier suitable for administration to animals including man. The peptides may be covalently attached to each other, to other peptides, to a protein carrier or to other carriers, incorporated into liposomes or other such vesicles, or complexed with an adjuvant or adsorbent as is known in the vaccine art. Alternatively, the peptides are not complexed with any of the above molecules and are merely admixed with a physiologically acceptable carrier such as normal saline or a buffering compound suitable for administration to animals including man. As with all immunogenic compositions for eliciting antibodies, the immunogenically effective amounts of the peptides of the invention must be determined. Factors to be considered include the immunogenicity of the native peptide, whether or not the peptide will be complexed with or covalently attached to an adjuvant or carrier protein or other carrier and route of administration for the composition, i.e. intravenous, intramuscular, subcutaneous, etc., and number of immunizing doses to be administered. Such factors are known in the vaccine art and it is well within the reach of a skilled artisan to make such determinations without undue experimentation. The peptides, parts or analogues thereof or compositions comprising these compounds may elicit an antibody response upon administrating to human or animal subjects. Such an antibody response protects against further infection by SARS-CoV and/or will retard the onset or progress of the symptoms associated with SARS.

Most preferably, they can be used in the treatment of a condition resulting from a SARS-CoV.

In yet another aspect, antibodies of the invention can be used as a medicament, preferably in the treatment of a condition resulting from a SARS-CoV. In a specific embodiment, they can be used with any other medicament available to treat a condition resulting from a SARS-CoV. In other words, the invention also pertains to a method of prevention and/or treatment, wherein the antibodies, fragments or functional variants thereof according to the invention are used.

The antibodies of the invention can also be used for detection of the SARS-CoV, e.g. for diagnostic purposes. Therefore, the invention provides a diagnostic test method for determining the presence of SARS-CoV in a sample, characterized in that the sample is put into contact with an antibody according to the invention. Preferably the antibody is contacted with the sample under conditions which allow the formation of an immunological complex between the antibodies and SARS-CoV or fragments or (poly)peptides thereof that may be present in the sample. The formation of an immunological complex, if any, indicating the presence of SARS-CoV in the sample, is then detected and measured by suitable means. The sample may be a biological sample including, but not limited to blood, serum, urine, tissue or other biological material from (potentially) infected subjects, or a nonbiological sample such as water, drink, etc. The (potentially) infected subjects may be human subjects, but also animals that are suspected as carriers of SARS-CoV might be tested for the presence of SARS-CoV using these antibodies. Detection of binding may be according to standard techniques known to a person skilled in the art, such as an ELISA, Western blot, RIA, etc. The antibodies may suitably be included in kits for diagnostic purposes. It is therefore another aspect of the invention to provide a kit of parts for the detection of SARS-CoV comprising an antibody according to the invention.

The antibodies of the invention may be used to purify SARS-CoV or a fragment thereof. Antibodies against peptides of the spike protein of SARS-CoV may also be used to purify the spike protein. Purification techniques for viruses and proteins are well known to the skilled artisan.

Also the peptide can be used directly for the detection of SARS-CoV recognizing antibodies, for instance for diagnostic purposes. It is therefore an object of the invention to provide methods for determining the presence of antibodies recognizing SARS-CoV in a sample, characterized in that the sample is put into contact with a peptide of the invention, preferably a peptide of the second embodiment described above. Preferably the peptide is contacted with the sample under conditions which allow the formation of an immunological complex between the peptide and any antibodies to SARS-CoV that may be present in the sample. The formation of an immunological complex, if any, indicating the presence of antibodies to SARS-CoV in the sample, is then detected and measured by suitable means. Such methods include, inter alia, homogeneous and heterogeneous binding immunoassays, such as radioimmunoassays (RIA), ELISA and Western blot analyses. Further, the assay protocols using the novel peptides allow for competitive and non-competitive binding studies to be performed. The sample used in the diagnostic test method may for instance be blood, tissue material or other material from potentially infected subjects. The peptide may however also be used to diagnose prior exposure to the SARS-CoV. Preferred assay techniques, especially for large-scale clinical screening of patient sera and blood and blood-derived products are ELISA and Western blot techniques. ELISA tests are particularly preferred. For use as reagents in these assays, the peptides of the invention are conveniently bonded to the inside surface of microtiter wells. The peptides may be directly bonded to the microtiter well. However, maximum binding of the peptides to the wells might be accomplished by pretreating the wells with polylysine prior to the addition of the peptides. Furthermore, the novel peptides may be covalently attached by known means to a carrier protein, such as BSA, with the resulting conjugate being used to coat the wells. Generally the peptides are used in a concentration of between 0.01 to 100 μg/ml for coating, although higher as well as lower amounts may also be used. Samples are then added to the peptide coated wells where an immunological complex forms if antibodies to SARS-CoV are present in the sample. A signal generating means may be added to aid detection of complex formation. A detectable signal is produced if SARS-CoV specific antibodies are present in the sample.

### EXAMPLES

PEPSCAN-ELISA

15-mer linear and looped/cyclic peptides were synthesized from the spike protein of SARS-CoV (see FIG. 2 and SEQ ID NO:39 for amino acid of surface spike glycoprotein of SARS-CoV; see also EMBL-datase accession number AY278741, “SARS coronavirus Urbani, complete genome.” The protein-id of the surface spike glycoprotein is AAP13441) and screened using credit-card format mini-PEPSCAN cards (455 peptide formats/card) as described previously (Slootstra et al., 1996; WO 93/09872). All peptides were acetylated at the amino terminus.

In all looped peptides position-2 and position-14 were replaced by a cysteine (acetyl-XCXXXXXXXXXXXCX-minicard). If other cysteines besides the cysteines at position-2 and position-14 were present in a prepared peptide, the other cysteines were replaced by an alanine. The looped peptides were synthesized using standard Fmoc-chemistry and deprotected using trifluoric acid with scavengers. Subsequently, the deprotected peptides were reacted on the cards with an 0.5 mM solution of 1,3-bis(bromomethyl)benzene in ammonium bicarbonate (20 mM, pH 7.9/acetonitril (1:1 (v/v)). The cards were gently shaken in the solution for 30-60 minutes, while completely covered in the solution. Finally, the cards were washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes.

The binding of antibodies to each linear and looped peptide was tested in a PEPSCAN-based enzyme-linked immuno assay (ELISA). The 455-well creditcard-format polypropylene cards, containing the covalently linked peptides, were incubated with serum (diluted 1/1000 in blocking solution which contains 5% horse-serum (v/v) and 5% ovalbumin (w/v)) (4° C., overnight). Before use, the serum was heat-inactivated at 56° C. for 1 hour. After washing the peptides were incubated with anti-human antibody peroxidase (dilution 1/1000) (1 hour, 25° C.), and subsequently, after washing the peroxidase substrate 2,2′-azino-di-3-ethylbenzthiazoline sulfonate (ABTS) and 2 μl/ml 3% H₂O₂ were added. After 1 hour the color development was measured. The color development of the ELISA was quantified with a CCD-camera and an image processing system. The setup consists of a CCD-camera and a 55 mm lens (Sony CCD Video Camera XC-77RR, Nikon micro-nikkor 55 mm f/2.8 lens), a camera adaptor (Sony Camera adaptor DC-77RR) and the Image Processing Software package Optimas, version 6.5 (Media Cybernetics, Silver Spring, Md. 20910, U.S.A.). Optimas runs on a pentium II computer system.

### RESULTS

The serum derived from an individual that has been infected by SARS-CoV and has recovered from SARS (serum called SARS-green) and the serum derived from an individual in which the virus was still detectable by PCR and who suffered a prolonged and severe form of the illness (serum called SARS-yellow) and the sera derived from individuals which have been and/or are still infected by SARS-CoV (the sera called 1a (1, early serum), 1b (1, late serum), 2, 6, 37, 62 and London were tested for binding to the 15-mer linear and looped/cyclic peptides synthesized as described supra. Additionally, two control sera were tested for binding the 15-mer linear and looped/cyclic peptides synthesized as described supra. One control serum was a pooled serum of 10 healthy LUMC (Leids Universitair Medisch Centrum) hospital workers and the second control serum was a commercial negative donor pooled serum from the Dutch blood bank. Next to that, a rabbit serum obtained by immunizing a rabbit with the SARS-CoV strain Frankfurt 1 was tested for binding the 15-mer linear and looped/cyclic peptides synthesized as described supra. The SARS-CoV was concentrated and partially purified by sucrose-gradient ultracentrifugation. After that, the purified SARS-CoV was gamma-irradiated for inactivation (approx. 35 kGy), mixed with complete Freund adjuvants and used for immunization purposes. Immunization was performed according to the art well known to the skilled artisan.

See Table 1 for results of the binding of the sera called SARS-yellow and SARS-green to the linear peptides of the spike protein of SARS-CoV Urbani. In Table 1 the SEQ ID NOs of the peptides are shown (see right column of Table 1).

See Table 2 for results of the binding of the different above sera to linear peptides of the spike protein of SARS-CoV Urbani. See Table 3 for results of the binding of the different above sera to looped/cyclic peptides of the spike protein of SARS-CoV Urbani.

See Table 4 for results of the binding of the two control sera to linear and looped/cyclic peptides of the spike protein of SARS-CoV Urbani. The following peptides were recognized by at least one of the control sera in linear form, looped/cyclic form or in both forms:

| | |
|---|---|
| VITPGTNASSEVAVL, | (SEQ ID NO:611) |
| ITPGTNASSEVAVLY, | (SEQ ID NO:612) |
| TPGTNASSEVAVLYQ, | (SEQ ID NO:613) |
| VSTAIHADQLTPAWR, | (SEQ ID NO:634) |
| STAIHADQLTPAWRI, | (SEQ ID NO:635) |
| TAIHADQLTPAWRIY, | (SEQ ID NO:636) |
| NTREVFAQVKQMYKT, | (SEQ ID NO:787) |
| TREVFAQVKQMYKTP, | (SEQ ID NO:788) |
| REVFAQVKQMYKTPT, | (SEQ ID NO:789) |
| EVFAQVKQMYKTPTL, | (SEQ ID NO:790) |
| VFAQVKQMYKTPTLK, | (SEQ ID NO:791) |
| FAQVKQMYKTPTLKY, | (SEQ ID NO:792) |
| AQVKQMYKTPTLKYF, | (SEQ ID NO:793) |
| QVKQMYKTPTLKYFG, | (SEQ ID NO:794) |
| SQILPDPLKPTKRSF, | (SEQ ID NO:813) |
| QILPDPLKPTKRSFI, | (SEQ ID NO:814) |
| ILPDPLKPTKRSFIE, | (SEQ ID NO:815) |
| LPDPLKPTKRSFIED, | (SEQ ID NO:816) |
| PDPLKPTKRSFIEDL, | (SEQ ID NO:817) |
| DPLKPTKRSFIEDLL, | (SEQ ID NO:818) |
| VLYENQKQIANQFNK, | (SEQ ID NO:925) |
| LYENQKQIANQFNKA, | (SEQ ID NO:926) |
| YENQKQIANQFNKAI, | (SEQ ID NO:927) |
| ENQKQIANQFNKAIS, | (SEQ ID NO:928) |
| NQKQIANQFNKAISQ, | (SEQ ID NO:929) |
| QKQIANQFNKAISQI, | (SEQ ID NO:930) |
| KQIANQFNKAISQIQ, | (SEQ ID NO:931) |
| QIANQFNKAISQIQE, | (SEQ ID NO:932) |
| IRAAEIRASANLAAT, | (SEQ ID NO:1023) |
| RAAEIRASANLAATK, | (SEQ ID NO:14) |
| AAEIRASANLAATKM, | (SEQ ID NO:15) |
| AEIRASANLAATKMS, | (SEQ ID NO:16) |
| SECVLGQSKRVDFCG, | (SEQ ID NO:1037) |
| ECVLGQSKRVDFCGK, | (SEQ ID NO:1038) |
| CVLGQSKRVDFCGKG, | (SEQ ID NO:1039) |
| VLGQSKRVDFCGKGY, | (SEQ ID NO:1040) |
| LGQSKRVDFCGKGYH, | (SEQ ID NO:1041) |
| GQSKRVDFCGKGYHL, | (SEQ ID NO:17) |
| QSKRVDFCGKGYHLM, | (SEQ ID NO:1042) |
| SKRVDFCGKGYHLMS, | (SEQ ID NO:1043) |
| YHLMSFPQAAPHGVV, | (SEQ ID NO:18) |
| HLMSFPQAAPHGVVF, | (SEQ ID NO:19) |
| LMSFPQAAPHGVVFL, | (SEQ ID NO:1053) |
| MSFPQAAPHGVVFLH, | (SEQ ID NO:1054) |
| SFPQAAPHGVVFLHV, | (SEQ ID NO:20) |
| FPQAAPHGVVFLHVT, | (SEQ ID NO:1055) |
| VTYVPSQERNFTTAP, | (SEQ ID NO:1068) |
| TYVPSQERNFTTAPA, | (SEQ ID NO:21) |
| YVPSQERNFTTAPAI, | (SEQ ID NO:22) |
| VPSQERNFTTAPAIC, | (SEQ ID NO:23) |
| PSQERNFTTAPAICH, | (SEQ ID NO:1069) |
| SQERNETTAPAICHE, | (SEQ ID NO:24) |
| QERNFTTAPAICHEG, | (SEQ ID NO:1070) |
| ERNFTTAPAICHEGK, | (SEQ ID NO:1071) |
| RNFTTAPAICHEGKA, | (SEQ ID NO:1072) |
| NFTTAPAICHEGKAY, | (SEQ ID NO:25) |
| FTTAPAICHEGKAYF, | (SEQ ID NO:1073) |
| TTAPAICHEGKAYFP, | (SEQ ID NO:1074) |
| TAPAICHEGKAYFPR, | (SEQ ID NO:1075) |
| APAICHEGKAYFPRE, | (SEQ ID NO:1076) |
| PAICHEGKAYFPREG, | (SEQ ID NO:1077) |
| TTDNTFVSGNCDVVI, | (SEQ ID NO:1114) |
| and | |
| TDNTFVSGNCDVVIG. | (SEQ ID NO:1115) |

In Table 5 the results of the binding of the rabbit serum to linear and looped/cyclic peptides of the spike protein of SARS-CoV Urbani are shown. The following peptides were recognized by the rabbit serum in linear form, looped/cyclic form or in both forms:

| | |
|---|---|
| LDRCTTFDDVQAPNY, | (SEQ ID NO:55) |
| DRCTTFDDVQAPNYT, | (SEQ ID NO:56) |
| RCTTFDDVQAPNYTQ, | (SEQ ID NO:57) |
| CTTFDDVQAPNYTQH, | (SEQ ID NO:58) |
| TTFDDVQAPNYTQHT, | (SEQ ID NO:59) |
| TFDDVQAPNYTQHTS, | (SEQ ID NO:60) |
| FDDVQAPNYTQHTSS, | (SEQ ID NO:61) |
| DDVQAPNYTQHTSSM, | (SEQ ID NO:62) |
| DVQAPNYTQHTSSMR, | (SEQ ID NO:63) |
| VQAPNYTQHTSSMRG, | (SEQ ID NO:64) |
| QAPNYTQHTSSMRGV, | (SEQ ID NO:65) |
| CDNPFFAVSKPMGTQ, | (SEQ ID NO:172) |
| DNPFFAVSKPMGTQT, | (SEQ ID NO:173) |
| NPFFAVSKPMGTQTH, | (SEQ ID NO:174) |
| PTTFMLKYDENGTIT, | (SEQ ID NO:287) |
| TTFMLKYDENGTITD, | (SEQ ID NO:288) |
| TFMLKYDENGTITDA, | (SEQ ID NO:289) |
| FMLKYDENGTITDAV, | (SEQ ID NO:290) |
| MLKYDENGTITDAVD, | (SEQ ID NO:291) |
| LKYDENGTITDAVDC, | (SEQ ID NO:292) |
| KYDENGTITDAVDCS, | (SEQ ID NO:293) |
| GIGYQPYRVVVLSFE, | (SEQ ID NO:516) |
| IGYQPYRVVVLSFEL, | (SEQ ID NO:517) |
| GYQPYRVVVLSFELL, | (SEQ ID NO:518) |
| SDFTDSVRDPKTSEI, | (SEQ ID NO:584) |
| DFTDSVRDPKTSEIL, | (SEQ ID NO:585) |
| FTDSVRDPKTSEILD, | (SEQ ID NO:586) |
| TDSVRDPKTSEILDI, | (SEQ ID NO:587) |
| DSVRDPKTSEILDIS, | (SEQ ID NO:588) |
| TTSTALGKLQDVVNQ, | (SEQ ID NO:950) |
| TSTALGKLQDVVNQN, | (SEQ ID NO:951) |
| STALGKLQDVVNQNA, | (SEQ ID NO:952) |
| TALGKLQDVVNQNAQ, | (SEQ ID NO:953) |
| ALGKLQDVVNQNAQA, | (SEQ ID NO:954) |
| LGKLQDVVNQNAQAL, | (SEQ ID NO:955) |
| GKLQDVVNQNAQALN, | (SEQ ID NO:956) |
| and | |
| KLQDVVNQNAQALNT. | (SEQ ID NO:957) |

The oligopeptides identified by the rabbit serum might be (additional) good candidates to represent epitopes of the SARS-CoV. The peptides may be advantageously used in diagnostic test methods as described herein. They may also be used in therapy and/or prevention of conditions resulting from an infection with SARS-CoV as described herein.

Relevant binding of a peptide to a serum was calculated as follows. The average OD-value for each serum was calculated for the spike protein (sum of OD-values of all peptides/total number of peptides). Next, the standard deviation of this average was calculated. The standard deviation was multiplied by 2 and the obtained value was added to the average value to obtain the correction factor. The OD-value of each peptide was then divided by this correction factor. If a value of 0.9 or higher was found, then relevant binding was considered to be present between the specific peptide and the respective serum. Particularly, domains (response of clustering of reactive peptides reactive with several individual sera) comprising several relevant peptides were claimed in the present invention. These domains are indicated (colored grey) in the the Tables 2, 3, 4 and 5.

Any of the above peptides could form the basis for diagnostic kits comprising the peptides, vaccines (as peptide, DNA, or vector vaccine) or for raising neutralizing antibodies to treat and/or prevent SARS.

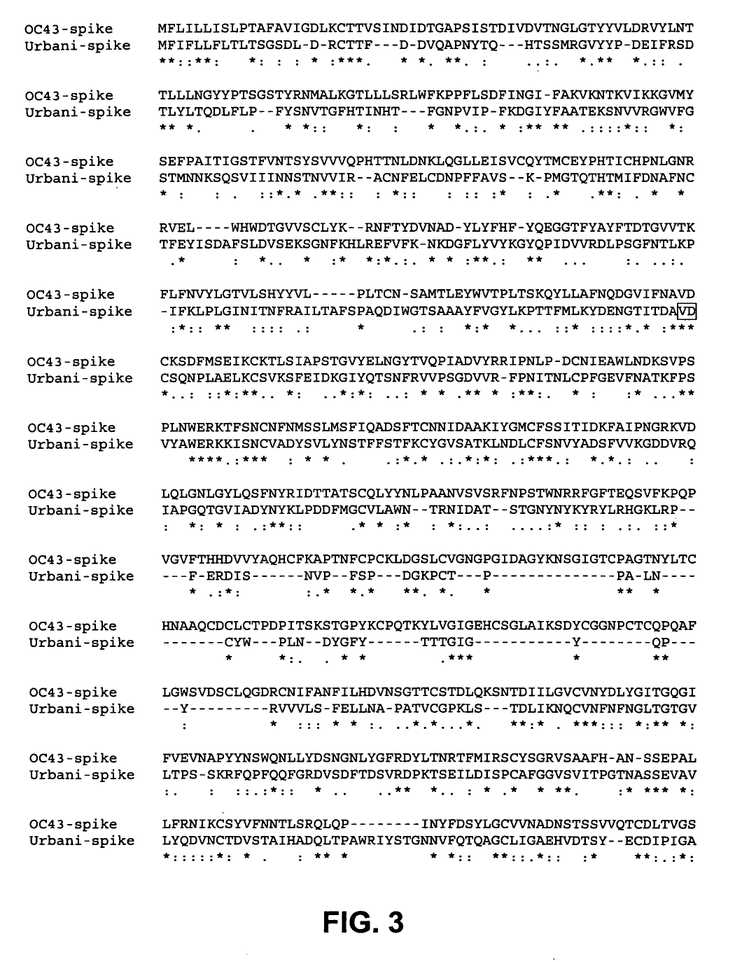

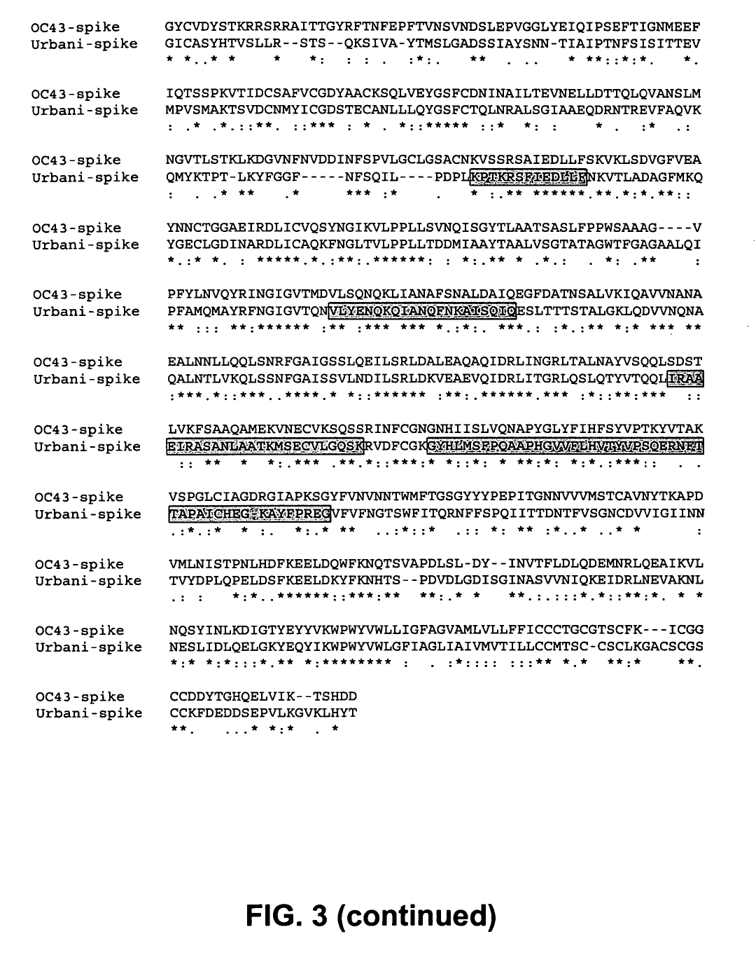

In FIG. 3 an alignment of the amino acid sequence of the spike glycoprotein of the human enteric coronavirus OC43 with the amino acid sequence of the spike protein of the SARS-CoV strain Urbani is shown. The human coronavirus OC43 gene for the surface protein can be found under the Genbank accession number Z32768 (see SEQ ID NO:1243 for the amino acid sequence of the spike glycoprotein of the human enteric coronavirus OC43). The alignment indicated that the C-terminal part of the spike glycoprotein of SARS-CoV strain Urbani showed high homology with the C terminal part of the spike glycoprotein of the human enteric coronavirus OC43. Besides that, the C-terminal part of the spike glycoprotein of the SARS-CoV strain Urbani contains several peptides recognized by sera derived from individuals which have been and/or are still infected by SARS-CoV (see Tables 1-3) and recognized by control sera derived from individuals which have not been infected by SARS-CoV (see Table 4). Based on these combined findings it is suggested that peptides of the spike glycoprotein of SARS-CoV recognized by sera derived from individuals which have been and/or are still infected by SARS-CoV and recognized by control sera derived from individuals which have not been infected by SARS-CoV, the peptides being highly homologous with corresponding peptides of other human coronaviruses, are useful for diagnosis, prevention and/or treatment of human coronavirus infections including, but not limited to, infections of SARS-CoV, OC43 and 22E. Peptides of the spike glycoprotein of SARS-CoV that fulfil all three requirements, i.e. a) recognition by sera derived from individuals which have been and/or are still infected by SARS-CoV, b) recognition by control sera derived from individuals which have not been infected by SARS-CoV, and c) being highly homologous with corresponding peptides of other human coronaviruses, such as for instance OC43, are indicated in colored boxes in FIG. 3. The identified peptides have the following amino acid sequences:

(SEQ ID NO:1244)

KPTKRSFIEDLLF,

(SEQ ID NO:1245)

VLYENQKQIANQFNKAISQIQ,

(SEQ ID NO:1246)

IRAAEIRASANLAATKMSECVLGQSK,
and

(SEQ ID NO:1247)

GYHLMSFPQAAPHGVVFLHVTYVPSQERNFTTAPAICHEGKAYFPREG.

It is clear for a skilled artisan that parts, variants or analogues of the peptides and peptides comprising the identified peptides are also a part of the invention.

Table 1: Binding of serum of infected and recovered patients to linear peptides of the spike protein of SARS-CoV. Applicants specifically incorporate by reference Table 1, in its entirety, from International Application No. PCT/EP2004/051102, filed Jun. 14, 2004, published in English as PCT International Publication No. WO 2004/111081 A2 on Dec. 23, 2004, located on pages 39-63 of the published application.

Table 2: Binding of the sera called SARS-yellow, SARS-green, 1a, 1b, 2, 6, 37, 62 and London to linear peptides of the spike protein of SARS-CoV Urbani. Applicants specifically incorporate by reference Table 2, in its entirety, from International Application No. PCT/EP2004/051102, filed Jun. 14, 2004, published in English as PCT International Publication No. WO 2004/111081 A2 on Dec. 23, 2004, located on pages 63-85 of the published application.

Table 3: Binding of the sera called SARS-yellow, SARS-green, 1a, 1b, 2, 6, 37, 62 and London to looped/cyclic peptides of the spike protein of SARS-CoV Urbani. Applicants specifically incorporate by reference Table 3, in its entirety, from International Application No. PCT/EP2004/051102, filed Jun. 14, 2004, published in English as PCT International Publication No. WO 2004/111081 A2 on Dec. 23, 2004, located on pages 85-108 of the published application.

Table 4: Binding of two control sera to linear and looped/cyclic peptides of the spike protein of SARS-CoV Urbani. Applicants specifically incorporate by reference Table 4, in its entirety, from International Application No. PCT/EP2004/051102, filed Jun. 14, 2004, published in English as PCT International Publication No. WO 2004/111081 A2 on Dec. 23, 2004, located on pages 108-131 of the published application.

Table 5: Binding of a rabbit serum to linear and looped/cyclic peptides of the spike protein of SARS-CoV Urbani. Applicants specifically incorporate by reference Table 5, in its entirety, from International Application No. PCT/EP2004/051102, filed Jun. 14, 2004, published in English as PCT International Publication No. WO 2004/111081 A2 on Dec. 23, 2004, located on pages 131-154 of the published application.

### REFERENCES

Holmes K. V. (2003) SARS coronavirus: a new challenge for prevention and therapy. J. Clin. Invest. 111, 1605-1609.
Ksiazek T. G., et al. (2003) A novel coronavirus associated with severe acute respiratory syndrome. N. Eng. J. Med. 348, 1953-1966.
Marra M. A., et al. (2003) The genome sequence of the SARS-associated coronavirus. Science 300, 1399-1404.
Posthumus W. P. A., et al. (1990) Analysis and simulation of a neutralizing epitope of Transmissible Gastroenteritis Virus. J. Virol. 64, 3304-3309.
Posthumus W. P. A., et al. (1991) Immunogenicity of peptides simulating a neutralization epitope of Transmissible Gastroenteritis Virus. Virol. 182, 371-375.
Rota P. A., et al. (2003) Characterization of a novel coronavirus associated with severe acute respiratory syndrome. Science 300, 1394-1399.
Slootstra J. W., et al. (1996) Structural aspects of antibody-antigen interaction revealed through small random peptide libraries. Mol. Divers. 1, 87-96.

1. An isolated oligopeptide consisting of an amino acid sequence selected from the group consisting: SEQ ID NO:40, SEQ ID NO:42, SEQ ID NO:44, SEQ ID NO:48-SEQ ID NO:51, SEQ ID NO:58, SEQ ID NO:70, SEQ ID NO:72, SEQ ID NO:75, SEQ ID NO:77, SEQ ID NO:107, SEQ ID NO:111 SEQ ID NO:112, SEQ ID NO:117, SEQ ID NO:118, SEQ ID NO:120, SEQ ID NO:123-SEQ ID NO:125, SEQ ID NO:127, SEQ ID NO:130, SEQ ID NO:135, SEQ ID NO:137, SEQ ID NO:138, SEQ ID NO:139, SEQ ID NO:141-SEQ ID NO: 143, SEQ ID NO:151, SEQ ID NO:153, SEQ ID NO:155, SEQ ID NO:156, SEQ ID NO:159-SEQ ID NO:164, SEQ ID NO:169, SEQ ID NO:173-SEQ ID NO:175, SEQ ID NO:179, SEQ ID NO:181, SEQ ID NO:184-SEQ ID NO:187, SEQ ID NO:189, SEQ ID NO:192-SEQ ID NO:194, SEQ ID NO:196, SEQ ID NO:197, SEQ ID NO:201-SEQ ID NO:203, SEQ ID NO:2, SEQ ID NO:4, SEQ ID NO:5, SEQ ID NO:7, SEQ ID NO:8, SEQ ID NO:11, SEQ ID NO:206, SEQ ID NO:208, SEQ ID NO:217-SEQ ID NO:219, SEQ ID NO:222, SEQ ID NO:225, SEQ ID NO:226, SEQ ID NO:231, SEQ ID NO:232, SEQ ID NO:236-SEQ ID NO:238, SEQ ID NO:246, SEQ ID NO:249, SEQ ID NO:257, SEQ ID NO:263), TAFSPAQDIWGTSAA (SEQ ID NO:264-SEQ ID NO:266, SEQ ID NO:268, SEQ ID NO:272, SEQ ID NO:273, SEQ ID NO:277, SEQ ID NO:279, SEQ ID NO:281-SEQ ID NO:284, SEQ ID NO:286-SEQ ID NO:293, SEQ ID NO:296-SEQ ID NO:314, SEQ ID NO:316, SEQ ID NO:323-SEQ ID NO:326, SEQ ID NO:330, SEQ ID NO:332, SEQ ID NO:336, SEQ ID NO:341, SEQ ID NO:345, SEQ ID NO:351, SEQ ID NO:354-SEQ ID NO:356, SEQ ID NO:360, SEQ ID NO:363, SEQ ID NO:366, SEQ ID NO:385, SEQ ID NO:390, SEQ ID NO:392-SEQ ID NO:395, SEQ ID NO:397, SEQ ID NO:399-SEQ ID NO:401, SEQ ID NO:411-SEQ ID NO:413, SEQ ID NO:415, SEQ ID NO:416, SEQ ID NO:421, SEQ ID NO:422, SEQ ID NO:425, SEQ ID NO:428-SEQ ID NO:430, SEQ ID NO:432-SEQ ID NO:436, SEQ ID NO:438, SEQ ID NO:441, SEQ ID NO:442, SEQ ID NO:444, SEQ ID NO:446-SEQ ID NO:448, SEQ ID NO:456, SEQ ID NO:461, SEQ ID NO:463, SEQ ID NO:464, SEQ ID NO:468, SEQ ID NO:469, SEQ ID NO:474, SEQ ID NO:484-SEQ ID NO:489, SEQ ID NO:494, SEQ ID NO:503, SEQ ID NO:504, SEQ ID NO:508, SEQ ID NO:509, SEQ ID NO:511-SEQ ID NO:515, SEQ BD NO:528, SEQ ID NO:529, SEQ ID NO:532, SEQ ID NO:534, SEQ ID NO:535, SEQ ID NO:538, SEQ ID NO:540, SEQ ID NO:542, SEQ ID NO:545, SEQ ID NO:547, SEQ ID NO:556-SEQ ID NO:559, SEQ ID NO:562-SEQ ID NO:570, SEQ ID NO:574, SEQ ID NO:578, SEQ ID NO:580, SEQ ID NO:582, SEQ ID NO:584-SEQ ID NO:586, SEQ ID NO:588-SEQ ID NO:594, SEQ ID NO:596, SEQ ID NO:597, SEQ ID NO:600-SEQ ID NO:602, SEQ ID NO:605-SEQ ID NO:609, SEQ ID NO:614-SEQ ID NO:616, SEQ ID NO:619, SEQ ID NO:621, SEQ ID NO:623-SEQ ID NO:634, -SEQ ID NO:640, SEQ ID NO:642, SEQ ID NO:647, SEQ ID NO:649-SEQ ID NO:651, SEQ ID NO:655-SEQ ID NO:665, SEQ ID NO:667, SEQ ID NO:670, SEQ ID NO:671, SEQ ID NO:673-SEQ ID NO:703, SEQ ID NO:705-SEQ ID NO:71 1, SEQ ID NO:714-SEQ ID NO:719, SEQ ID NO:724-SEQ ID NO:730, SEQ ID NO:732-SEQ ID NO:734, SEQ ID NO:736-EQ ID NO:738, SEQ ID NO:741-EQ ID NO:754, SEQ ID NO:756, SEQ ID NO:762, SEQ ID NO:765, SEQ ID NO:766, SEQ ID NO:768, SEQ ID NO:776-SEQ ID NO:778, SEQ ID NO:780, SEQ ID NO:781, SEQ ID NO:784-SEQ ID NO:786, SEQ ID NO:791, SEQ ID NO:792, SEQ ID NO:795, SEQ ID NO:796, SEQ ID NO:798, SEQ ID NO:800, SEQ ID NO:804, SEQ ID NO:806, SEQ ID NO:808, SEQ ID NO:810-SEQ ID NO:812, SEQ ID NO:817, SEQ ID NO:819-SEQ ID NO:825, SEQ ID NO:827, SEQ ID NO:828, SEQ ID NO:831, SEQ ID NO:833, SEQ ID NO:835, SEQ ID NO:836, SEQ ID NO:839, SEQ ID NO:842-SEQ ID NO:844, SEQ ID NO:846-SEQ ID NO:859, SEQ ID NO:866, SEQ ID NO:871, SEQ ID NO:873, SEQ ID NO:877, SEQ ID NO:879, SEQ ID NO:880, SEQ ID NO:882, SEQ ID NO:886-SEQ ID NO:889, SEQ ID NO:891-SEQ ID NO:893, SEQ ID NO:896, SEQ ID NO:898 SEQ ID NO:899, SEQ ID NO:904, SEQ ID NO:905, SEQ ID NO:909, SEQ ID NO:919, SEQ ID NO:921-SEQ ID NO:924, SEQ ID NO:938, SEQ ID NO:939, SEQ ID NO:947, SEQ ID NO:955-SEQ ID NO:957, SEQ ID NO:964, SEQ ID NO:966, SEQ ID NO:968, SEQ ID NO:981, SEQ ID NO:982, SEQ ID NO:983, SEQ ID NO:993, SEQ ID NO:994, SEQ ID NO:999, SEQ ID NO:1009-SEQ ID NO:1012, SEQ ID NO:1017, SEQ ID NO:1024, SEQ ID NO:1036, SEQ ID NO:1048, SEQ ID NO:1049, SEQ ID NO:1052, SEQ ID NO:1056, SEQ ID NO:1060, SEQ ID NO:1063, SEQ ID NO:1067, SEQ ID NO:1070, SEQ ID NO:1074, SEQ ID NO:1079-SEQ ID NO:1081, SEQ ID NO:1083, SEQ ID NO:1085, SEQ ID NO:1086, SEQ ID NO:1088, SEQ ID NO:1091, SEQ ID NO:1094, SEQ ID NO:1102-SEQ ID NO:1105, SEQ ID NO:1107, SEQ ID NO:1108, SEQ ID NO:1112, SEQ ID NO:1116, SEQ ID NO:1118, SEQ ID NO:1123, SEQ ID NO:1127, SEQ ID NO:1128, SEQ ID NO:1135, SEQ ID NO:1136, SEQ ID NO:1137, SEQ ID NO:1141, SEQ ID NO:1144-SEQ ID NO:1146, SEQ ID NO:12, SEQ ID NO:13, SEQ ID NO:1147, SEQ ID NO:1149, SEQ ID NO:1152, SEQ ID NO:1153, SEQ ID NO:1156, SEQ ID NO:1160, SEQ ID NO:1165, SEQ ID NO:1167-SEQ ID NO:1170, SEQ ID NO:1172, SEQ ID NO:1179-SEQ ID NO:1183, SEQ ID NO:1185, SEQ ID NO:1186, SEQ ID NO:1188, SEQ ID NO:1190-SEQ ID NO:1192, SEQ ID NO:1194, SEQ ID NO:1229, SEQ ID NO:1232, SEQ ID NO:1234, SEQ ID NO:26-SEQ ID NO:28, SEQ ID NO:30, SEQ ID NO:31, SEQ ID NO:35, SEQ ID NO:36-SEQ ID NO:38,SEQ ID NO:1238, and SEQ ID NO:1242.

2. An isolated peptide consisting of an amino acid sequence selected from the group consisting of: SEQ ID NO: 1248-SEQ ID NO: 1264.

3. An isolated peptide consisting of an analogue of the peptide of claim 1, wherein one or more amino acids are substituted, inserted, deleted or combinations thereof, and wherein said analogue is recognized by antibodies present in serum derived from an individual that has been or is infected by SARS-CoV.

4. An isolated peptide consisting of an amino acid sequence selected from the group consisting of: SEQ ID NO:56-SEQ ID NO:65, SEQ ID NO:516-SEQ ID NO:518, and SEQ ID NO:953.

5. An isolated peptide consisting of an analogue of the peptide of claim 4, wherein one or more amino acids are substituted, inserted, deleted or combinations thereof, and wherein said analogue is recognized by antibodies present in serum derived from a subject that has been immunized with inactivated SARS-CoV.

6. An isolated fusion protein comprising the peptide of claim 1.

7. An isolated nucleic acid molecule encoding the peptide of claim 1.

8. An isolated antibody capable of binding to the peptide of claim 1.

9. The isolated antibody of claim 8, wherein said antibody is a monoclonal antibody.

10. The isolated antibody of claim 9, wherein said monoclonal antibody is a human monoclonal antibody.

11. The isolated antibody of claim 8, characterized in that the antibody has SARS-CoV neutralizing activity.

12. An isolated nucleic acid molecule encoding the antibody of claim 8.

13. A vector comprising at least one nucleic acid molecule of claim 7.

14. A host comprising at least one vector of claim 13.

15. The host of claim 14, wherein the host is a cell.

16. (canceled)

17. An immunogenic composition comprising the isolated oligopeptide of claim 1 and a carrier or diluent.

18. An immunogenic composition comprising an oligopeptide means for recognition by antibodies present in serum from a subject that has been or is infected by SARS-CoV and a carrier or diluent.

19. An immunogenic composition comprising the isolated oligopeptide of claim 2 and a carrier or diluent.

20-22. (canceled)

23. A diagnostic test method for determining the presence of an antibody recognizing SARS-CoV in a sample, said method comprising the steps of:
contacting said sample with the peptide of claim 1 and
determining whether the antibody in the sample binds to said peptide.

24. A diagnostic test method for determining the presence of a SARS-CoV molecule in a sample, said method comprising the steps of:
contacting said sample with the antibody of claim 8 and
determining whether the antibody binds to the SARS-CoV molecule.

25. The diagnostic test method of claim 23, wherein the sample is from a human subject

26. A method for determining a neutralizing epitope on a protein of an infectious agent causing disease in a human, said method comprising the steps of
comparing the binding of epitopes on said protein by serum or antibodies drawn from a first human infected with said infectious agent that has not fully recovered, or had a prolonged or more severe disease history as compared with the disease history of a second human, with serum or antibodies drawn from the second human infected by said infectious agent that has fully recovered and/or had a less prolonged and/or less severe disease history as compared with the disease history of the first human and
determining which epitope on the protein of the infectious agent has a greater binding affinity with the serum or antibodies of the second human.

27. The method of claim 26, characterized in that the infectious agent is a virus.

28. The method of claim 27, characterized in that the infectious agent is SARS-CoV.

29. The method of claim 28, characterized in that the protein is the S protein.

30. An isolated nucleic acid molecule encoding the fusion protein of claim 7.

31. An isolated peptide consisting of an analogue of the peptide of claim 2, wherein one or more amino acids are substituted, inserted, deleted or combinations thereof, and wherein said analogue is recognized by antibodies present in serum derived from an individual that has been or is infected by SARS-CoV.

32. An isolated antibody capable of binding to the fusion protein of claim 7.

33. A vector comprising at least one nucleic acid molecule of claim 13.

34. A host cell comprising at least one vector of claim 32.

35. A method for treating or preventing a condition resulting from a SARS-CoV in a subject, said method comprising the step of administering an effective amount of a medicament comprising the antibody of claim 8 to the subject.

36. A diagnostic test method for determining the presence of an antibody recognizing SARS-CoV in a sample, said method comprising the steps of:
contacting said sample with the fusion protein of claim 6 and
determining whether the antibody in the sample binds to said peptide.


SEQUENCE LISTING (1264 sequences)

SEQ ID NO: 1 - protein, 15 aa, Artificial sequence
  Synthetic peptide
DAFSLDVSEKSGNFK

SEQ ID NO: 2 - protein, 15 aa, Artificial sequence
  Synthetic peptide
AFSLDVSEKSGNFKH

SEQ ID NO: 3 - protein, 15 aa, Artificial sequence
  Synthetic peptide
FSLDVSEKSGNFKHL

SEQ ID NO: 4 - protein, 15 aa, Artificial sequence
  Synthetic peptide
SLDVSEKSGNFKHLR

SEQ ID NO: 5 - protein, 15 aa, Artificial sequence
  Synthetic peptide
LDVSEKSGNFKHLRE

SEQ ID NO: 6 - protein, 15 aa, Artificial sequence
  Synthetic peptide
DVSEKSGNFKHLREF

SEQ ID NO: 7 - protein, 15 aa, Artificial sequence
  Synthetic peptide
VSEKSGNFKHLREFV

SEQ ID NO: 8 - protein, 15 aa, Artificial sequence
  Synthetic peptide
SEKSGNFKHLREFVF

SEQ ID NO: 9 - protein, 15 aa, Artificial sequence
  Synthetic peptide
EKSGNFKHLREFVFK

SEQ ID NO: 10 - protein, 15 aa, Artificial sequence
  Synthetic peptide
KSGNFKHLREFVFKN

SEQ ID NO: 11 - protein, 15 aa, Artificial sequence
  Synthetic peptide
SGNFKHLREFVFKNK

SEQ ID NO: 12 - protein, 15 aa, Artificial sequence
  Synthetic peptide
KEELDKYFKNHTSPD

SEQ ID NO: 13 - protein, 15 aa, Artificial sequence
  Synthetic peptide
EELDKYFKNHTSPDV

SEQ ID NO: 14 - protein, 15 aa, Artificial sequence
  Synthetic peptide
RAAEIRASANLAATK

SEQ ID NO: 15 - protein, 15 aa, Artificial sequence
  Synthetic peptide
AAEIRASANLAATKM

SEQ ID NO: 16 - protein, 15 aa, Artificial sequence
  Synthetic peptide
AEIRASANLAATKMS

SEQ ID NO: 17 - protein, 15 aa, Artificial sequence
  Synthetic peptide
GQSKRVDFCGKGYHL

SEQ ID NO: 18 - protein, 15 aa, Artificial sequence
  Synthetic peptide
YHLMSFPQAAPHGVV

SEQ ID NO: 19 - protein, 15 aa, Artificial sequence
  Synthetic peptide
HLMSFPQAAPHGVVF

SEQ ID NO: 20 - protein, 15 aa, Artificial sequence
  Synthetic peptide
SFPQAAPHGVVFLHV

SEQ ID NO: 21 - protein, 15 aa, Artificial sequence
  Synthetic peptide
TYVPSQERNFTTAPA

SEQ ID NO: 22 - protein, 15 aa, Artificial sequence
  Synthetic peptide
YVPSQERNFTTAPAI

SEQ ID NO: 23 - protein, 15 aa, Artificial sequence
  Synthetic peptide
VPSQERNFTTAPAIC

SEQ ID NO: 24 - protein, 15 aa, Artificial sequence
  Synthetic peptide
SQERNFTTAPAICHE

SEQ ID NO: 25 - protein, 15 aa, Artificial sequence
  Synthetic peptide
NFTTAPAICHEGKAY

SEQ ID NO: 26 - protein, 15 aa, Artificial sequence
  Synthetic peptide
SCCSCLKGACSCGSC

SEQ ID NO: 27 - protein, 15 aa, Artificial sequence
  Synthetic peptide
CCSCLKGACSCGSCC

SEQ ID NO: 28 - protein, 15 aa, Artificial sequence
  Synthetic peptide
CSCLKGACSCGSCCK

SEQ ID NO: 29 - protein, 15 aa, Artificial sequence
  Synthetic peptide
SCLKGACSCGSCCKF

SEQ ID NO: 30 - protein, 15 aa, Artificial sequence
  Synthetic peptide
CLKGACSCGSCCKFD

SEQ ID NO: 31 - protein, 15 aa, Artificial sequence
  Synthetic peptide
LKGACSCGSCCKFDE

SEQ ID NO: 32 - protein, 15 aa, Artificial sequence
  Synthetic peptide
KGACSCGSCCKFDED

SEQ ID NO: 33 - protein, 15 aa, Artificial sequence
  Synthetic peptide
GACSCGSCCKFDEDD

SEQ ID NO: 34 - protein, 15 aa, Artificial sequence
  Synthetic peptide
ACSCGSCCKFDEDDS

SEQ ID NO: 35 - protein, 15 aa, Artificial sequence
  Synthetic peptide
CSCGSCCKFDEDDSE

SEQ ID NO: 36 - protein, 15 aa, Artificial sequence
  Synthetic peptide
SCGSCCKFDEDDSEP

SEQ ID NO: 37 - protein, 15 aa, Artificial sequence
  Synthetic peptide
CGSCCKFDEDDSEPV

SEQ ID NO: 38 - protein, 15 aa, Artificial sequence
  Synthetic peptide
GSCCKFDEDDSEPVL

SEQ ID NO: 39 - protein, 1255 aa, SARS-CoV
  MISC_FEATURE: Amino acid sequence of surface spike glycoprotein of SARS-CoV
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 40 - protein, 15 aa, Artificial sequence
  Synthetic peptide
MFIFLLFLTLTSGSD

SEQ ID NO: 41 - protein, 15 aa, Artificial sequence
  Synthetic peptide
FIFLLFLTLTSGSDL

SEQ ID NO: 42 - protein, 15 aa, Artificial sequence
  Synthetic peptide
IFLLFLTLTSGSDLD

SEQ ID NO: 43 - protein, 15 aa, Artificial sequence
  Synthetic peptide
FLLFLTLTSGSDLDR

SEQ ID NO: 44 - protein, 15 aa, Artificial sequence
  Synthetic peptide
LLFLTLTSGSDLDRC

SEQ ID NO: 45 - protein, 15 aa, Artificial sequence
  Synthetic peptide
LFLTLTSGSDLDRCT

SEQ ID NO: 46 - protein, 15 aa, Artificial sequence
  Synthetic peptide
FLTLTSGSDLDRCTT

SEQ ID NO: 47 - protein, 15 aa, Artificial sequence
  Synthetic peptide
LTLTSGSDLDRCTTF

SEQ ID NO: 48 - protein, 15 aa, Artificial sequence
  Synthetic peptide
TLTSGSDLDRCTTFD

SEQ ID NO: 49 - protein, 15 aa, Artificial sequence
  Synthetic peptide
LTSGSDLDRCTTFDD

SEQ ID NO: 50 - protein, 15 aa, Artificial sequence
  Synthetic peptide
TSGSDLDRCTTFDDV

SEQ ID NO: 51 - protein, 15 aa, Artificial sequence
  Synthetic peptide
SGSDLDRCTTFDDVQ

SEQ ID NO: 52 - protein, 15 aa, Artificial sequence
  Synthetic peptide
GSDLDRCTTFDDVQA

SEQ ID NO: 53 - protein, 15 aa, Artificial sequence
  Synthetic peptide
SDLDRCTTFDDVQAP

SEQ ID NO: 54 - protein, 15 aa, Artificial sequence
  Synthetic peptide
DLDRCTTFDDVQAPN

SEQ ID NO: 55 - protein, 15 aa, Artificial sequence
  Synthetic peptide
LDRCTTFDDVQAPNY

SEQ ID NO: 56 - protein, 15 aa, Artificial sequence
  Synthetic peptide
DRCTTFDDVQAPNYT

SEQ ID NO: 57 - protein, 15 aa, Artificial sequence
  Synthetic peptide
RCTTFDDVQAPNYTQ

SEQ ID NO: 58 - protein, 15 aa, Artificial sequence
  Synthetic peptide
CTTFDDVQAPNYTQH

SEQ ID NO: 59 - protein, 15 aa, Artificial sequence
  Synthetic peptide
TTFDDVQAPNYTQHT

SEQ ID NO: 60 - protein, 15 aa, Artificial sequence
  Synthetic peptide
TFDDVQAPNYTQHTS

SEQ ID NO: 61 - protein, 15 aa, Artificial sequence
  Synthetic peptide
FDDVQAPNYTQHTSS

SEQ ID NO: 62 - protein, 15 aa, Artificial sequence
  Synthetic peptide
DDVQAPNYTQHTSSM

SEQ ID NO: 63 - protein, 15 aa, Artificial sequence
  Synthetic peptide
DVQAPNYTQHTSSMR

SEQ ID NO: 64 - protein, 15 aa, Artificial sequence
  Synthetic peptide
VQAPNYTQHTSSMRG

SEQ ID NO: 65 - protein, 15 aa, Artificial sequence
  Synthetic peptide
QAPNYTQHTSSMRGV

SEQ ID NO: 66 - protein, 15 aa, Artificial sequence
  Synthetic peptide
APNYTQHTSSMRGVY

SEQ ID NO: 67 - protein, 15 aa, Artificial sequence
  Synthetic peptide
PNYTQHTSSMRGVYY

SEQ ID NO: 68 - protein, 15 aa, Artificial sequence
  Synthetic peptide
NYTQHTSSMRGVYYP

SEQ ID NO: 69 - protein, 15 aa, Artificial sequence
  Synthetic peptide
YTQHTSSMRGVYYPD

SEQ ID NO: 70 - protein, 15 aa, Artificial sequence
  Synthetic peptide
TQHTSSMRGVYYPDE

SEQ ID NO: 71 - protein, 15 aa, Artificial sequence
  Synthetic peptide
QHTSSMRGVYYPDEI

SEQ ID NO: 72 - protein, 15 aa, Artificial sequence
  Synthetic peptide
HTSSMRGVYYPDEIF

SEQ ID NO: 73 - protein, 15 aa, Artificial sequence
  Synthetic peptide
TSSMRGVYYPDEIFR

SEQ ID NO: 74 - protein, 15 aa, Artificial sequence
  Synthetic peptide
SSMRGVYYPDEIFRS

SEQ ID NO: 75 - protein, 15 aa, Artificial sequence
  Synthetic peptide
SMRGVYYPDEIFRSD

SEQ ID NO: 76 - protein, 15 aa, Artificial sequence
  Synthetic peptide
MRGVYYPDEIFRSDT

SEQ ID NO: 77 - protein, 15 aa, Artificial sequence
  Synthetic peptide
RGVYYPDEIFRSDTL

SEQ ID NO: 78 - protein, 15 aa, Artificial sequence
  Synthetic peptide
GVYYPDEIFRSDTLY

SEQ ID NO: 79 - protein, 15 aa, Artificial sequence
  Synthetic peptide
VYYPDEIFRSDTLYL

SEQ ID NO: 80 - protein, 15 aa, Artificial sequence
  Synthetic peptide
YYPDEIFRSDTLYLT

SEQ ID NO: 81 - protein, 15 aa, Artificial sequence
  Synthetic peptide
YPDEIFRSDTLYLTQ

SEQ ID NO: 82 - protein, 15 aa, Artificial sequence
  Synthetic peptide
PDEIFRSDTLYLTQD

SEQ ID NO: 83 - protein, 15 aa, Artificial sequence
  Synthetic peptide
DEIFRSDTLYLTQDL

SEQ ID NO: 84 - protein, 15 aa, Artificial sequence
  Synthetic peptide
EIFRSDTLYLTQDLF

SEQ ID NO: 85 - protein, 15 aa, Artificial sequence
  Synthetic peptide
IFRSDTLYLTQDLFL

SEQ ID NO: 86 - protein, 15 aa, Artificial sequence
  Synthetic peptide
FRSDTLYLTQDLFLP

SEQ ID NO: 87 - protein, 15 aa, Artificial sequence
  Synthetic peptide
RSDTLYLTQDLFLPF

SEQ ID NO: 88 - protein, 15 aa, Artificial sequence
  Synthetic peptide
SDTLYLTQDLFLPFY

SEQ ID NO: 89 - protein, 15 aa, Artificial sequence
  Synthetic peptide
DTLYLTQDLFLPFYS

SEQ ID NO: 90 - protein, 15 aa, Artificial sequence
  Synthetic peptide
TLYLTQDLFLPFYSN

SEQ ID NO: 91 - protein, 15 aa, Artificial sequence
  Synthetic peptide
LYLTQDLFLPFYSNV

SEQ ID NO: 92 - protein, 15 aa, Artificial sequence
  Synthetic peptide
YLTQDLFLPFYSNVT

SEQ ID NO: 93 - protein, 15 aa, Artificial sequence
  Synthetic peptide
LTQDLFLPFYSNVTG

SEQ ID NO: 94 - protein, 15 aa, Artificial sequence
  Synthetic peptide
TQDLFLPFYSNVTGF

SEQ ID NO: 95 - protein, 15 aa, Artificial sequence
  Synthetic peptide
QDLFLPFYSNVTGFH

SEQ ID NO: 96 - protein, 15 aa, Artificial sequence
  Synthetic peptide
DLFLPFYSNVTGFHT

SEQ ID NO: 97 - protein, 15 aa, Artificial sequence
  Synthetic peptide
LFLPFYSNVTGFHTI

SEQ ID NO: 98 - protein, 15 aa, Artificial sequence
  Synthetic peptide
FLPFYSNVTGFHTIN

SEQ ID NO: 99 - protein, 15 aa, Artificial sequence
  Synthetic peptide
LPFYSNVTGFHTINH

SEQ ID NO: 100 - protein, 15 aa, Artificial sequence
  Synthetic peptide
PFYSNVTGFHTINHT

SEQ ID NO: 101 - protein, 15 aa, Artificial sequence
  Synthetic peptide
FYSNVTGFHTINHTF

SEQ ID NO: 102 - protein, 15 aa, Artificial sequence
  Synthetic peptide
YSNVTGFHTINHTFG

SEQ ID NO: 103 - protein, 15 aa, Artificial sequence
  Synthetic peptide
SNVTGFHTINHTFGN

SEQ ID NO: 104 - protein, 15 aa, Artificial sequence
  Synthetic peptide
NVTGFHTINHTFGNP

SEQ ID NO: 105 - protein, 15 aa, Artificial sequence
  Synthetic peptide
VTGFHTINHTFGNPV

SEQ ID NO: 106 - protein, 15 aa, Artificial sequence
  Synthetic peptide
TGFHTINHTFGNPVI

SEQ ID NO: 107 - protein, 15 aa, Artificial sequence
  Synthetic peptide
GFHTINHTFGNPVIP

SEQ ID NO: 108 - protein, 15 aa, Artificial sequence
  Synthetic peptide
FHTINHTFGNPVIPF

SEQ ID NO: 109 - protein, 15 aa, Artificial sequence
  Synthetic peptide
HTINHTFGNPVIPFK

SEQ ID NO: 110 - protein, 15 aa, Artificial sequence
  Synthetic peptide
TINHTFGNPVIPFKD

SEQ ID NO: 111 - protein, 15 aa, Artificial sequence
  Synthetic peptide
INHTFGNPVIPFKDG

SEQ ID NO: 112 - protein, 15 aa, Artificial sequence
  Synthetic peptide
NHTFGNPVIPFKDGI

SEQ ID NO: 113 - protein, 15 aa, Artificial sequence
  Synthetic peptide
HTFGNPVIPFKDGIY

SEQ ID NO: 114 - protein, 15 aa, Artificial sequence
  Synthetic peptide
TFGNPVIPFKDGIYF

SEQ ID NO: 115 - protein, 15 aa, Artificial sequence
  Synthetic peptide
FGNPVIPFKDGIYFA

SEQ ID NO: 116 - protein, 15 aa, Artificial sequence
  Synthetic peptide
GNPVIPFKDGIYFAA

SEQ ID NO: 117 - protein, 15 aa, Artificial sequence
  Synthetic peptide
NPVIPFKDGIYFAAT

SEQ ID NO: 118 - protein, 15 aa, Artificial sequence
  Synthetic peptide
PVIPFKDGIYFAATE

SEQ ID NO: 119 - protein, 15 aa, Artificial sequence
  Synthetic peptide
VIPFKDGIYFAATEK

SEQ ID NO: 120 - protein, 15 aa, Artificial sequence
  Synthetic peptide
IPFKDGIYFAATEKS

SEQ ID NO: 121 - protein, 15 aa, Artificial sequence
  Synthetic peptide
PFKDGIYFAATEKSN

SEQ ID NO: 122 - protein, 15 aa, Artificial sequence
  Synthetic peptide
FKDGIYFAATEKSNV

SEQ ID NO: 123 - protein, 15 aa, Artificial sequence
  Synthetic peptide
KDGIYFAATEKSNVV

SEQ ID NO: 124 - protein, 15 aa, Artificial sequence
  Synthetic peptide
DGIYFAATEKSNVVR

SEQ ID NO: 125 - protein, 15 aa, Artificial sequence
  Synthetic peptide
GIYFAATEKSNVVRG

SEQ ID NO: 126 - protein, 15 aa, Artificial sequence
  Synthetic peptide
IYFAATEKSNVVRGW

SEQ ID NO: 127 - protein, 15 aa, Artificial sequence
  Synthetic peptide
YFAATEKSNVVRGWV

SEQ ID NO: 128 - protein, 15 aa, Artificial sequence
  Synthetic peptide
FAATEKSNVVRGWVF

SEQ ID NO: 129 - protein, 15 aa, Artificial sequence
  Synthetic peptide
AATEKSNVVRGWVFG

SEQ ID NO: 130 - protein, 15 aa, Artificial sequence
  Synthetic peptide
ATEKSNVVRGWVFGS

SEQ ID NO: 131 - protein, 15 aa, Artificial sequence
  Synthetic peptide
TEKSNVVRGWVFGST

SEQ ID NO: 132 - protein, 15 aa, Artificial sequence
  Synthetic peptide
EKSNVVRGWVFGSTM

SEQ ID NO: 133 - protein, 15 aa, Artificial sequence
  Synthetic peptide
KSNVVRGWVFGSTMN

SEQ ID NO: 134 - protein, 15 aa, Artificial sequence
  Synthetic peptide
SNVVRGWVFGSTMNN

SEQ ID NO: 135 - protein, 15 aa, Artificial sequence
  Synthetic peptide
NVVRGWVFGSTMNNK

SEQ ID NO: 136 - protein, 15 aa, Artificial sequence
  Synthetic peptide
VVRGWVFGSTMNNKS

SEQ ID NO: 137 - protein, 15 aa, Artificial sequence
  Synthetic peptide
VRGWVFGSTMNNKSQ

SEQ ID NO: 138 - protein, 15 aa, Artificial sequence
  Synthetic peptide
RGWVFGSTMNNKSQS

SEQ ID NO: 139 - protein, 15 aa, Artificial sequence
  Synthetic peptide
GWVFGSTMNNKSQSV

SEQ ID NO: 140 - protein, 15 aa, Artificial sequence
  Synthetic peptide
WVFGSTMNNKSQSVI

SEQ ID NO: 141 - protein, 15 aa, Artificial sequence
  Synthetic peptide
VFGSTMNNKSQSVII

SEQ ID NO: 142 - protein, 15 aa, Artificial sequence
  Synthetic peptide
FGSTMNNKSQSVIII

SEQ ID NO: 143 - protein, 15 aa, Artificial sequence
  Synthetic peptide
GSTMNNKSQSVIIIN

SEQ ID NO: 144 - protein, 15 aa, Artificial sequence
  Synthetic peptide
STMNNKSQSVIIINN

SEQ ID NO: 145 - protein, 15 aa, Artificial sequence
  Synthetic peptide
TMNNKSQSVIIINNS

SEQ ID NO: 146 - protein, 15 aa, Artificial sequence
  Synthetic peptide
MNNKSQSVIIINNST

SEQ ID NO: 147 - protein, 15 aa, Artificial sequence
  Synthetic peptide
NNKSQSVIIINNSTN

SEQ ID NO: 148 - protein, 15 aa, Artificial sequence
  Synthetic peptide
NKSQSVIIINNSTNV

SEQ ID NO: 149 - protein, 15 aa, Artificial sequence
  Synthetic peptide
KSQSVIIINNSTNVV

SEQ ID NO: 150 - protein, 15 aa, Artificial sequence
  Synthetic peptide
SQSVIIINNSTNVVI

SEQ ID NO: 151 - protein, 15 aa, Artificial sequence
  Synthetic peptide
QSVIIINNSTNVVIR

SEQ ID NO: 152 - protein, 15 aa, Artificial sequence
  Synthetic peptide
SVIIINNSTNVVIRA

SEQ ID NO: 153 - protein, 15 aa, Artificial sequence
  Synthetic peptide
VIIINNSTNVVIRAC

SEQ ID NO: 154 - protein, 15 aa, Artificial sequence
  Synthetic peptide
IIINNSTNVVIRACN

SEQ ID NO: 155 - protein, 15 aa, Artificial sequence
  Synthetic peptide
IINNSTNVVIRACNF

SEQ ID NO: 156 - protein, 15 aa, Artificial sequence
  Synthetic peptide
INNSTNVVIRACNFE

SEQ ID NO: 157 - protein, 15 aa, Artificial sequence
  Synthetic peptide
NNSTNVVIRACNFEL

SEQ ID NO: 158 - protein, 15 aa, Artificial sequence
  Synthetic peptide
NSTNVVIRACNFELC

SEQ ID NO: 159 - protein, 15 aa, Artificial sequence
  Synthetic peptide
STNVVIRACNFELCD

SEQ ID NO: 160 - protein, 15 aa, Artificial sequence
  Synthetic peptide
TNVVIRACNFELCDN

SEQ ID NO: 161 - protein, 15 aa, Artificial sequence
  Synthetic peptide
NVVIRACNFELCDNP

SEQ ID NO: 162 - protein, 15 aa, Artificial sequence
  Synthetic peptide
VVIRACNFELCDNPF

SEQ ID NO: 163 - protein, 15 aa, Artificial sequence
  Synthetic peptide
VIRACNFELCDNPFF

SEQ ID NO: 164 - protein, 15 aa, Artificial sequence
  Synthetic peptide
IRACNFELCDNPFFA

SEQ ID NO: 165 - protein, 15 aa, Artificial sequence
  Synthetic peptide
RACNFELCDNPFFAV

SEQ ID NO: 166 - protein, 15 aa, Artificial sequence
  Synthetic peptide
ACNFELCDNPFFAVS

SEQ ID NO: 167 - protein, 15 aa, Artificial sequence
  Synthetic peptide
CNFELCDNPFFAVSK

SEQ ID NO: 168 - protein, 15 aa, Artificial sequence
  Synthetic peptide
NFELCDNPFFAVSKP

SEQ ID NO: 169 - protein, 15 aa, Artificial sequence
  Synthetic peptide
FELCDNPFFAVSKPM

SEQ ID NO: 170 - protein, 15 aa, Artificial sequence
  Synthetic peptide
ELCDNPFFAVSKPMG

SEQ ID NO: 171 - protein, 15 aa, Artificial sequence
  Synthetic peptide
LCDNPFFAVSKPMGT

SEQ ID NO: 172 - protein, 15 aa, Artificial sequence
  Synthetic peptide
CDNPFFAVSKPMGTQ

SEQ ID NO: 173 - protein, 15 aa, Artificial sequence
  Synthetic peptide
DNPFFAVSKPMGTQT

SEQ ID NO: 174 - protein, 15 aa, Artificial sequence
  Synthetic peptide
NPFFAVSKPMGTQTH

SEQ ID NO: 175 - protein, 15 aa, Artificial sequence
  Synthetic peptide
PFFAVSKPMGTQTHT

SEQ ID NO: 176 - protein, 15 aa, Artificial sequence
  Synthetic peptide
FFAVSKPMGTQTHTM

SEQ ID NO: 177 - protein, 15 aa, Artificial sequence
  Synthetic peptide
FAVSKPMGTQTHTMI

SEQ ID NO: 178 - protein, 15 aa, Artificial sequence
  Synthetic peptide
AVSKPMGTQTHTMIF

SEQ ID NO: 179 - protein, 15 aa, Artificial sequence
  Synthetic peptide
VSKPMGTQTHTMIFD

SEQ ID NO: 180 - protein, 15 aa, Artificial sequence
  Synthetic peptide
SKPMGTQTHTMIFDN

SEQ ID NO: 181 - protein, 15 aa, Artificial sequence
  Synthetic peptide
KPMGTQTHTMIFDNA

SEQ ID NO: 182 - protein, 15 aa, Artificial sequence
  Synthetic peptide
PMGTQTHTMIFDNAF

SEQ ID NO: 183 - protein, 15 aa, Artificial sequence
  Synthetic peptide
MGTQTHTMIFDNAFN

SEQ ID NO: 184 - protein, 15 aa, Artificial sequence
  Synthetic peptide
GTQTHTMIFDNAFNC

SEQ ID NO: 185 - protein, 15 aa, Artificial sequence
  Synthetic peptide
TQTHTMIFDNAFNCT

SEQ ID NO: 186 - protein, 15 aa, Artificial sequence
  Synthetic peptide
QTHTMIFDNAFNCTF

SEQ ID NO: 187 - protein, 15 aa, Artificial sequence
  Synthetic peptide
THTMIFDNAFNCTFE

SEQ ID NO: 188 - protein, 15 aa, Artificial sequence
  Synthetic peptide
HTMIFDNAFNCTFEY

SEQ ID NO: 189 - protein, 15 aa, Artificial sequence
  Synthetic peptide
TMIFDNAFNCTFEYI

SEQ ID NO: 190 - protein, 15 aa, Artificial sequence
  Synthetic peptide
MIFDNAFNCTFEYIS

SEQ ID NO: 191 - protein, 15 aa, Artificial sequence
  Synthetic peptide
IFDNAFNCTFEYISD

SEQ ID NO: 192 - protein, 15 aa, Artificial sequence
  Synthetic peptide
FDNAFNCTFEYISDA

SEQ ID NO: 193 - protein, 15 aa, Artificial sequence
  Synthetic peptide
DNAFNCTFEYISDAF

SEQ ID NO: 194 - protein, 15 aa, Artificial sequence
  Synthetic peptide
NAFNCTFEYISDAFS

SEQ ID NO: 195 - protein, 15 aa, Artificial sequence
  Synthetic peptide
AFNCTFEYISDAFSL

SEQ ID NO: 196 - protein, 15 aa, Artificial sequence
  Synthetic peptide
FNCTFEYISDAFSLD

SEQ ID NO: 197 - protein, 15 aa, Artificial sequence
  Synthetic peptide
NCTFEYISDAFSLDV

SEQ ID NO: 198 - protein, 15 aa, Artificial sequence
  Synthetic peptide
CTFEYISDAFSLDVS

SEQ ID NO: 199 - protein, 15 aa, Artificial sequence
  Synthetic peptide
TFEYISDAFSLDVSE

SEQ ID NO: 200 - protein, 15 aa, Artificial sequence
  Synthetic peptide
FEYISDAFSLDVSEK

SEQ ID NO: 201 - protein, 15 aa, Artificial sequence
  Synthetic peptide
EYISDAFSLDVSEKS

SEQ ID NO: 202 - protein, 15 aa, Artificial sequence
  Synthetic peptide
YISDAFSLDVSEKSG

SEQ ID NO: 203 - protein, 15 aa, Artificial sequence
  Synthetic peptide
ISDAFSLDVSEKSGN

SEQ ID NO: 204 - protein, 15 aa, Artificial sequence
  Synthetic peptide
SDAFSLDVSEKSGNF

SEQ ID NO: 205 - protein, 15 aa, Artificial sequence
  Synthetic peptide
GNFKHLREFVFKNKD

SEQ ID NO: 206 - protein, 15 aa, Artificial sequence
  Synthetic peptide
NFKHLREFVFKNKDG

SEQ ID NO: 207 - protein, 15 aa, Artificial sequence
  Synthetic peptide
FKHLREFVFKNKDGF

SEQ ID NO: 208 - protein, 15 aa, Artificial sequence
  Synthetic peptide
KHLREFVFKNKDGFL

SEQ ID NO: 209 - protein, 15 aa, Artificial sequence
  Synthetic peptide
HLREFVFKNKDGFLY

SEQ ID NO: 210 - protein, 15 aa, Artificial sequence
  Synthetic peptide
LREFVFKNKDGFLYV

SEQ ID NO: 211 - protein, 15 aa, Artificial sequence
  Synthetic peptide
REFVFKNKDGFLYVY

SEQ ID NO: 212 - protein, 15 aa, Artificial sequence
  Synthetic peptide
EFVFKNKDGFLYVYK

SEQ ID NO: 213 - protein, 15 aa, Artificial sequence
  Synthetic peptide
FVFKNKDGFLYVYKG

SEQ ID NO: 214 - protein, 15 aa, Artificial sequence
  Synthetic peptide
VFKNKDGFLYVYKGY

SEQ ID NO: 215 - protein, 15 aa, Artificial sequence
  Synthetic peptide
FKNKDGFLYVYKGYQ

SEQ ID NO: 216 - protein, 15 aa, Artificial sequence
  Synthetic peptide
KNKDGFLYVYKGYQP

SEQ ID NO: 217 - protein, 15 aa, Artificial sequence
  Synthetic peptide
NKDGFLYVYKGYQPI

SEQ ID NO: 218 - protein, 15 aa, Artificial sequence
  Synthetic peptide
KDGFLYVYKGYQPID

SEQ ID NO: 219 - protein, 15 aa, Artificial sequence
  Synthetic peptide
DGFLYVYKGYQPIDV

SEQ ID NO: 220 - protein, 15 aa, Artificial sequence
  Synthetic peptide
GFLYVYKGYQPIDVV

SEQ ID NO: 221 - protein, 15 aa, Artificial sequence
  Synthetic peptide
FLYVYKGYQPIDVVR

SEQ ID NO: 222 - protein, 15 aa, Artificial sequence
  Synthetic peptide
LYVYKGYQPIDVVRD

SEQ ID NO: 223 - protein, 15 aa, Artificial sequence
  Synthetic peptide
YVYKGYQPIDVVRDL

SEQ ID NO: 224 - protein, 15 aa, Artificial sequence
  Synthetic peptide
VYKGYQPIDVVRDLP

SEQ ID NO: 225 - protein, 15 aa, Artificial sequence
  Synthetic peptide
YKGYQPIDVVRDLPS

SEQ ID NO: 226 - protein, 15 aa, Artificial sequence
  Synthetic peptide
KGYQPIDVVRDLPSG

SEQ ID NO: 227 - protein, 15 aa, Artificial sequence
  Synthetic peptide
GYQPIDVVRDLPSGF

SEQ ID NO: 228 - protein, 15 aa, Artificial sequence
  Synthetic peptide
YQPIDVVRDLPSGFN

SEQ ID NO: 229 - protein, 15 aa, Artificial sequence
  Synthetic peptide
QPIDVVRDLPSGFNT

SEQ ID NO: 230 - protein, 15 aa, Artificial sequence
  Synthetic peptide
PIDVVRDLPSGFNTL

SEQ ID NO: 231 - protein, 15 aa, Artificial sequence
  Synthetic peptide
IDVVRDLPSGFNTLK

SEQ ID NO: 232 - protein, 15 aa, Artificial sequence
  Synthetic peptide
DVVRDLPSGFNTLKP

SEQ ID NO: 233 - protein, 15 aa, Artificial sequence
  Synthetic peptide
VVRDLPSGFNTLKPI

SEQ ID NO: 234 - protein, 15 aa, Artificial sequence
  Synthetic peptide
VRDLPSGFNTLKPIF

SEQ ID NO: 235 - protein, 15 aa, Artificial sequence
  Synthetic peptide
RDLPSGFNTLKPIFK

SEQ ID NO: 236 - protein, 15 aa, Artificial sequence
  Synthetic peptide
DLPSGFNTLKPIFKL

SEQ ID NO: 237 - protein, 15 aa, Artificial sequence
  Synthetic peptide
LPSGFNTLKPIFKLP

SEQ ID NO: 238 - protein, 15 aa, Artificial sequence
  Synthetic peptide
PSGFNTLKPIFKLPL

SEQ ID NO: 239 - protein, 15 aa, Artificial sequence
  Synthetic peptide
SGFNTLKPIFKLPLG

SEQ ID NO: 240 - protein, 15 aa, Artificial sequence
  Synthetic peptide
GFNTLKPIFKLPLGI

SEQ ID NO: 241 - protein, 15 aa, Artificial sequence
  Synthetic peptide
FNTLKPIFKLPLGIN

SEQ ID NO: 242 - protein, 15 aa, Artificial sequence
  Synthetic peptide
NTLKPIFKLPLGINI

SEQ ID NO: 243 - protein, 15 aa, Artificial sequence
  Synthetic peptide
TLKPIFKLPLGINIT

SEQ ID NO: 244 - protein, 15 aa, Artificial sequence
  Synthetic peptide
LKPIFKLPLGINITN

SEQ ID NO: 245 - protein, 15 aa, Artificial sequence
  Synthetic peptide
KPIFKLPLGINITNF

SEQ ID NO: 246 - protein, 15 aa, Artificial sequence
  Synthetic peptide
PIFKLPLGINITNFR

SEQ ID NO: 247 - protein, 15 aa, Artificial sequence
  Synthetic peptide
IFKLPLGINITNFRA

SEQ ID NO: 248 - protein, 15 aa, Artificial sequence
  Synthetic peptide
FKLPLGINITNFRAI

SEQ ID NO: 249 - protein, 15 aa, Artificial sequence
  Synthetic peptide
KLPLGINITNFRAIL

SEQ ID NO: 250 - protein, 15 aa, Artificial sequence
  Synthetic peptide
LPLGINITNFRAILT

SEQ ID NO: 251 - protein, 15 aa, Artificial sequence
  Synthetic peptide
PLGINITNFRAILTA

SEQ ID NO: 252 - protein, 15 aa, Artificial sequence
  Synthetic peptide
LGINITNFRAILTAF

SEQ ID NO: 253 - protein, 15 aa, Artificial sequence
  Synthetic peptide
GINITNFRAILTAFS

SEQ ID NO: 254 - protein, 15 aa, Artificial sequence
  Synthetic peptide
INITNFRAILTAFSP

SEQ ID NO: 255 - protein, 15 aa, Artificial sequence
  Synthetic peptide
NITNFRAILTAFSPA

SEQ ID NO: 256 - protein, 15 aa, Artificial sequence
  Synthetic peptide
ITNFRAILTAFSPAQ

SEQ ID NO: 257 - protein, 15 aa, Artificial sequence
  Synthetic peptide
TNFRAILTAFSPAQD

SEQ ID NO: 258 - protein, 15 aa, Artificial sequence
  Synthetic peptide
NFRAILTAFSPAQDI

SEQ ID NO: 259 - protein, 15 aa, Artificial sequence
  Synthetic peptide
FRAILTAFSPAQDIW

SEQ ID NO: 260 - protein, 15 aa, Artificial sequence
  Synthetic peptide
RAILTAFSPAQDIWG

SEQ ID NO: 261 - protein, 15 aa, Artificial sequence
  Synthetic peptide
AILTAFSPAQDIWGT

SEQ ID NO: 262 - protein, 15 aa, Artificial sequence
  Synthetic peptide
ILTAFSPAQDIWGTS

SEQ ID NO: 263 - protein, 15 aa, Artificial sequence
  Synthetic peptide
LTAFSPAQDIWGTSA

SEQ ID NO: 264 - protein, 15 aa, Artificial sequence
  Synthetic peptide
TAFSPAQDIWGTSAA

SEQ ID NO: 265 - protein, 15 aa, Artificial sequence
  Synthetic peptide
AFSPAQDIWGTSAAA

SEQ ID NO: 266 - protein, 15 aa, Artificial sequence
  Synthetic peptide
FSPAQDIWGTSAAAY

SEQ ID NO: 267 - protein, 15 aa, Artificial sequence
  Synthetic peptide
SPAQDIWGTSAAAYF

SEQ ID NO: 268 - protein, 15 aa, Artificial sequence
  Synthetic peptide
PAQDIWGTSAAAYFV

SEQ ID NO: 269 - protein, 15 aa, Artificial sequence
  Synthetic peptide
AQDIWGTSAAAYFVG

SEQ ID NO: 270 - protein, 15 aa, Artificial sequence
  Synthetic peptide
QDIWGTSAAAYFVGY

SEQ ID NO: 271 - protein, 15 aa, Artificial sequence
  Synthetic peptide
DIWGTSAAAYFVGYL

SEQ ID NO: 272 - protein, 15 aa, Artificial sequence
  Synthetic peptide
IWGTSAAAYFVGYLK

SEQ ID NO: 273 - protein, 15 aa, Artificial sequence
  Synthetic peptide
WGTSAAAYFVGYLKP

SEQ ID NO: 274 - protein, 15 aa, Artificial sequence
  Synthetic peptide
GTSAAAYFVGYLKPT

SEQ ID NO: 275 - protein, 15 aa, Artificial sequence
  Synthetic peptide
TSAAAYFVGYLKPTT

SEQ ID NO: 276 - protein, 15 aa, Artificial sequence
  Synthetic peptide
SAAAYFVGYLKPTTF

SEQ ID NO: 277 - protein, 15 aa, Artificial sequence
  Synthetic peptide
AAAYFVGYLKPTTFM

SEQ ID NO: 278 - protein, 15 aa, Artificial sequence
  Synthetic peptide
AAYFVGYLKPTTFML

SEQ ID NO: 279 - protein, 15 aa, Artificial sequence
  Synthetic peptide
AYFVGYLKPTTFMLK

SEQ ID NO: 280 - protein, 15 aa, Artificial sequence
  Synthetic peptide
YFVGYLKPTTFMLKY

SEQ ID NO: 281 - protein, 15 aa, Artificial sequence
  Synthetic peptide
FVGYLKPTTFMLKYD

SEQ ID NO: 282 - protein, 15 aa, Artificial sequence
  Synthetic peptide
VGYLKPTTFMLKYDE

SEQ ID NO: 283 - protein, 15 aa, Artificial sequence
  Synthetic peptide
GYLKPTTFMLKYDEN

SEQ ID NO: 284 - protein, 15 aa, Artificial sequence
  Synthetic peptide
YLKPTTFMLKYDENG

SEQ ID NO: 285 - protein, 15 aa, Artificial sequence
  Synthetic peptide
LKPTTFMLKYDENGT

SEQ ID NO: 286 - protein, 15 aa, Artificial sequence
  Synthetic peptide
KPTTFMLKYDENGTI

SEQ ID NO: 287 - protein, 15 aa, Artificial sequence
  Synthetic peptide
PTTFMLKYDENGTIT

SEQ ID NO: 288 - protein, 15 aa, Artificial sequence
  Synthetic peptide
TTFMLKYDENGTITD

SEQ ID NO: 289 - protein, 15 aa, Artificial sequence
  Synthetic peptide
TFMLKYDENGTITDA

SEQ ID NO: 290 - protein, 15 aa, Artificial sequence
  Synthetic peptide
FMLKYDENGTITDAV

SEQ ID NO: 291 - protein, 15 aa, Artificial sequence
  Synthetic peptide
MLKYDENGTITDAVD

SEQ ID NO: 292 - protein, 15 aa, Artificial sequence
  Synthetic peptide
LKYDENGTITDAVDC

SEQ ID NO: 293 - protein, 15 aa, Artificial sequence
  Synthetic peptide
KYDENGTITDAVDCS

SEQ ID NO: 294 - protein, 15 aa, Artificial sequence
  Synthetic peptide
YDENGTITDAVDCSQ

SEQ ID NO: 295 - protein, 15 aa, Artificial sequence
  Synthetic peptide
DENGTITDAVDCSQN

SEQ ID NO: 296 - protein, 15 aa, Artificial sequence
  Synthetic peptide
ENGTITDAVDCSQNP

SEQ ID NO: 297 - protein, 15 aa, Artificial sequence
  Synthetic peptide
NGTITDAVDCSQNPL

SEQ ID NO: 298 - protein, 15 aa, Artificial sequence
  Synthetic peptide
GTITDAVDCSQNPLA

SEQ ID NO: 299 - protein, 15 aa, Artificial sequence
  Synthetic peptide
TITDAVDCSQNPLAE

SEQ ID NO: 300 - protein, 15 aa, Artificial sequence
  Synthetic peptide
ITDAVDCSQNPLAEL

SEQ ID NO: 301 - protein, 15 aa, Artificial sequence
  Synthetic peptide
TDAVDCSQNPLAELK

SEQ ID NO: 302 - protein, 15 aa, Artificial sequence
  Synthetic peptide
DAVDCSQNPLAELKC

SEQ ID NO: 303 - protein, 15 aa, Artificial sequence
  Synthetic peptide
AVDCSQNPLAELKCS

SEQ ID NO: 304 - protein, 15 aa, Artificial sequence
  Synthetic peptide
VDCSQNPLAELKCSV

SEQ ID NO: 305 - protein, 15 aa, Artificial sequence
  Synthetic peptide
DCSQNPLAELKCSVK

SEQ ID NO: 306 - protein, 15 aa, Artificial sequence
  Synthetic peptide
CSQNPLAELKCSVKS

SEQ ID NO: 307 - protein, 15 aa, Artificial sequence
  Synthetic peptide
SQNPLAELKCSVKSF

SEQ ID NO: 308 - protein, 15 aa, Artificial sequence
  Synthetic peptide
QNPLAELKCSVKSFE

SEQ ID NO: 309 - protein, 15 aa, Artificial sequence
  Synthetic peptide
NPLAELKCSVKSFEI

SEQ ID NO: 310 - protein, 15 aa, Artificial sequence
  Synthetic peptide
PLAELKCSVKSFEID

SEQ ID NO: 311 - protein, 15 aa, Artificial sequence
  Synthetic peptide
LAELKCSVKSFEIDK

SEQ ID NO: 312 - protein, 15 aa, Artificial sequence
  Synthetic peptide
AELKCSVKSFEIDKG

SEQ ID NO: 313 - protein, 15 aa, Artificial sequence
  Synthetic peptide
ELKCSVKSFEIDKGI

SEQ ID NO: 314 - protein, 15 aa, Artificial sequence
  Synthetic peptide
LKCSVKSFEIDKGIY

SEQ ID NO: 315 - protein, 15 aa, Artificial sequence
  Synthetic peptide
KCSVKSFEIDKGIYQ

SEQ ID NO: 316 - protein, 15 aa, Artificial sequence
  Synthetic peptide
CSVKSFEIDKGIYQT

SEQ ID NO: 317 - protein, 15 aa, Artificial sequence
  Synthetic peptide
SVKSFEIDKGIYQTS

SEQ ID NO: 318 - protein, 15 aa, Artificial sequence
  Synthetic peptide
VKSFEIDKGIYQTSN

SEQ ID NO: 319 - protein, 15 aa, Artificial sequence
  Synthetic peptide
KSFEIDKGIYQTSNF

SEQ ID NO: 320 - protein, 15 aa, Artificial sequence
  Synthetic peptide
SFEIDKGIYQTSNFR

SEQ ID NO: 321 - protein, 15 aa, Artificial sequence
  Synthetic peptide
FEIDKGIYQTSNFRV

SEQ ID NO: 322 - protein, 15 aa, Artificial sequence
  Synthetic peptide
EIDKGIYQTSNFRVV

SEQ ID NO: 323 - protein, 15 aa, Artificial sequence
  Synthetic peptide
IDKGIYQTSNFRVVP

SEQ ID NO: 324 - protein, 15 aa, Artificial sequence
  Synthetic peptide
DKGIYQTSNFRVVPS

SEQ ID NO: 325 - protein, 15 aa, Artificial sequence
  Synthetic peptide
KGIYQTSNFRVVPSG

SEQ ID NO: 326 - protein, 15 aa, Artificial sequence
  Synthetic peptide
GIYQTSNFRVVPSGD

SEQ ID NO: 327 - protein, 15 aa, Artificial sequence
  Synthetic peptide
IYQTSNFRVVPSGDV

SEQ ID NO: 328 - protein, 15 aa, Artificial sequence
  Synthetic peptide
YQTSNFRVVPSGDVV

SEQ ID NO: 329 - protein, 15 aa, Artificial sequence
  Synthetic peptide
QTSNFRVVPSGDVVR

SEQ ID NO: 330 - protein, 15 aa, Artificial sequence
  Synthetic peptide
TSNFRVVPSGDVVRF

SEQ ID NO: 331 - protein, 15 aa, Artificial sequence
  Synthetic peptide
SNFRVVPSGDVVRFP

SEQ ID NO: 332 - protein, 15 aa, Artificial sequence
  Synthetic peptide
NFRVVPSGDVVRFPN

SEQ ID NO: 333 - protein, 15 aa, Artificial sequence
  Synthetic peptide
FRVVPSGDVVRFPNI

SEQ ID NO: 334 - protein, 15 aa, Artificial sequence
  Synthetic peptide
RVVPSGDVVRFPNIT

SEQ ID NO: 335 - protein, 15 aa, Artificial sequence
  Synthetic peptide
VVPSGDVVRFPNITN

SEQ ID NO: 336 - protein, 15 aa, Artificial sequence
  Synthetic peptide
VPSGDVVRFPNITNL

SEQ ID NO: 337 - protein, 15 aa, Artificial sequence
  Synthetic peptide
PSGDVVRFPNITNLC

SEQ ID NO: 338 - protein, 15 aa, Artificial sequence
  Synthetic peptide
SGDVVRFPNITNLCP

SEQ ID NO: 339 - protein, 15 aa, Artificial sequence
  Synthetic peptide
GDVVRFPNITNLCPF

SEQ ID NO: 340 - protein, 15 aa, Artificial sequence
  Synthetic peptide
DVVRFPNITNLCPFG

SEQ ID NO: 341 - protein, 15 aa, Artificial sequence
  Synthetic peptide
VVRFPNITNLCPFGE

SEQ ID NO: 342 - protein, 15 aa, Artificial sequence
  Synthetic peptide
VRFPNITNLCPFGEV

SEQ ID NO: 343 - protein, 15 aa, Artificial sequence
  Synthetic peptide
RFPNITNLCPFGEVF

SEQ ID NO: 344 - protein, 15 aa, Artificial sequence
  Synthetic peptide
FPNITNLCPFGEVFN

SEQ ID NO: 345 - protein, 15 aa, Artificial sequence
  Synthetic peptide
PNITNLCPFGEVFNA

SEQ ID NO: 346 - protein, 15 aa, Artificial sequence
  Synthetic peptide
NITNLCPFGEVFNAT

SEQ ID NO: 347 - protein, 15 aa, Artificial sequence
  Synthetic peptide
ITNLCPFGEVFNATK

SEQ ID NO: 348 - protein, 15 aa, Artificial sequence
  Synthetic peptide
TNLCPFGEVFNATKF

SEQ ID NO: 349 - protein, 15 aa, Artificial sequence
  Synthetic peptide
NLCPFGEVFNATKFP

SEQ ID NO: 350 - protein, 15 aa, Artificial sequence
  Synthetic peptide
LCPFGEVFNATKFPS

SEQ ID NO: 351 - protein, 15 aa, Artificial sequence
  Synthetic peptide
CPFGEVFNATKFPSV

SEQ ID NO: 352 - protein, 15 aa, Artificial sequence
  Synthetic peptide
PFGEVFNATKFPSVY

SEQ ID NO: 353 - protein, 15 aa, Artificial sequence
  Synthetic peptide
FGEVFNATKFPSVYA

SEQ ID NO: 354 - protein, 15 aa, Artificial sequence
  Synthetic peptide
GEVFNATKFPSVYAW

SEQ ID NO: 355 - protein, 15 aa, Artificial sequence
  Synthetic peptide
EVFNATKFPSVYAWE

SEQ ID NO: 356 - protein, 15 aa, Artificial sequence
  Synthetic peptide
VFNATKFPSVYAWER

SEQ ID NO: 357 - protein, 15 aa, Artificial sequence
  Synthetic peptide
FNATKFPSVYAWERK

SEQ ID NO: 358 - protein, 15 aa, Artificial sequence
  Synthetic peptide
NATKFPSVYAWERKK

SEQ ID NO: 359 - protein, 15 aa, Artificial sequence
  Synthetic peptide
ATKFPSVYAWERKKI

SEQ ID NO: 360 - protein, 15 aa, Artificial sequence
  Synthetic peptide
TKFPSVYAWERKKIS

SEQ ID NO: 361 - protein, 15 aa, Artificial sequence
  Synthetic peptide
KFPSVYAWERKKISN

SEQ ID NO: 362 - protein, 15 aa, Artificial sequence
  Synthetic peptide
FPSVYAWERKKISNC

SEQ ID NO: 363 - protein, 15 aa, Artificial sequence
  Synthetic peptide
PSVYAWERKKISNCV

SEQ ID NO: 364 - protein, 15 aa, Artificial sequence
  Synthetic peptide
SVYAWERKKISNCVA

SEQ ID NO: 365 - protein, 15 aa, Artificial sequence
  Synthetic peptide
VYAWERKKISNCVAD

SEQ ID NO: 366 - protein, 15 aa, Artificial sequence
  Synthetic peptide
YAWERKKISNCVADY

SEQ ID NO: 367 - protein, 15 aa, Artificial sequence
  Synthetic peptide
AWERKKISNCVADYS

SEQ ID NO: 368 - protein, 15 aa, Artificial sequence
  Synthetic peptide
WERKKISNCVADYSV

SEQ ID NO: 369 - protein, 15 aa, Artificial sequence
  Synthetic peptide
ERKKISNCVADYSVL

SEQ ID NO: 370 - protein, 15 aa, Artificial sequence
  Synthetic peptide
RKKISNCVADYSVLY

SEQ ID NO: 371 - protein, 15 aa, Artificial sequence
  Synthetic peptide
KKISNCVADYSVLYN

SEQ ID NO: 372 - protein, 15 aa, Artificial sequence
  Synthetic peptide
KISNCVADYSVLYNS

SEQ ID NO: 373 - protein, 15 aa, Artificial sequence
  Synthetic peptide
ISNCVADYSVLYNST

SEQ ID NO: 374 - protein, 15 aa, Artificial sequence
  Synthetic peptide
SNCVADYSVLYNSTF

SEQ ID NO: 375 - protein, 15 aa, Artificial sequence
  Synthetic peptide
NCVADYSVLYNSTFF

SEQ ID NO: 376 - protein, 15 aa, Artificial sequence
  Synthetic peptide
CVADYSVLYNSTFFS

SEQ ID NO: 377 - protein, 15 aa, Artificial sequence
  Synthetic peptide
VADYSVLYNSTFFST

SEQ ID NO: 378 - protein, 15 aa, Artificial sequence
  Synthetic peptide
ADYSVLYNSTFFSTF

SEQ ID NO: 379 - protein, 15 aa, Artificial sequence
  Synthetic peptide
DYSVLYNSTFFSTFK

SEQ ID NO: 380 - protein, 15 aa, Artificial sequence
  Synthetic peptide
YSVLYNSTFFSTFKC

SEQ ID NO: 381 - protein, 15 aa, Artificial sequence
  Synthetic peptide
SVLYNSTFFSTFKCY

SEQ ID NO: 382 - protein, 15 aa, Artificial sequence
  Synthetic peptide
VLYNSTFFSTFKCYG

SEQ ID NO: 383 - protein, 15 aa, Artificial sequence
  Synthetic peptide
LYNSTFFSTFKCYGV

SEQ ID NO: 384 - protein, 15 aa, Artificial sequence
  Synthetic peptide
YNSTFFSTFKCYGVS

SEQ ID NO: 385 - protein, 15 aa, Artificial sequence
  Synthetic peptide
NSTFFSTFKCYGVSA

SEQ ID NO: 386 - protein, 15 aa, Artificial sequence
  Synthetic peptide
STFFSTFKCYGVSAT

SEQ ID NO: 387 - protein, 15 aa, Artificial sequence
  Synthetic peptide
TFFSTFKCYGVSATK

SEQ ID NO: 388 - protein, 15 aa, Artificial sequence
  Synthetic peptide
FFSTFKCYGVSATKL

SEQ ID NO: 389 - protein, 15 aa, Artificial sequence
  Synthetic peptide
FSTFKCYGVSATKLN

SEQ ID NO: 390 - protein, 15 aa, Artificial sequence
  Synthetic peptide
STFKCYGVSATKLND

SEQ ID NO: 391 - protein, 15 aa, Artificial sequence
  Synthetic peptide
TFKCYGVSATKLNDL

SEQ ID NO: 392 - protein, 15 aa, Artificial sequence
  Synthetic peptide
FKCYGVSATKLNDLC

SEQ ID NO: 393 - protein, 15 aa, Artificial sequence
  Synthetic peptide
KCYGVSATKLNDLCF

SEQ ID NO: 394 - protein, 15 aa, Artificial sequence
  Synthetic peptide
CYGVSATKLNDLCFS

SEQ ID NO: 395 - protein, 15 aa, Artificial sequence
  Synthetic peptide
YGVSATKLNDLCFSN

SEQ ID NO: 396 - protein, 15 aa, Artificial sequence
  Synthetic peptide
GVSATKLNDLCFSNV

SEQ ID NO: 397 - protein, 15 aa, Artificial sequence
  Synthetic peptide
VSATKLNDLCFSNVY

SEQ ID NO: 398 - protein, 15 aa, Artificial sequence
  Synthetic peptide
SATKLNDLCFSNVYA

SEQ ID NO: 399 - protein, 15 aa, Artificial sequence
  Synthetic peptide
ATKLNDLCFSNVYAD

SEQ ID NO: 400 - protein, 15 aa, Artificial sequence
  Synthetic peptide
TKLNDLCFSNVYADS

SEQ ID NO: 401 - protein, 15 aa, Artificial sequence
  Synthetic peptide
KLNDLCFSNVYADSF

SEQ ID NO: 402 - protein, 15 aa, Artificial sequence
  Synthetic peptide
LNDLCFSNVYADSFV

SEQ ID NO: 403 - protein, 15 aa, Artificial sequence
  Synthetic peptide
NDLCFSNVYADSFVV

SEQ ID NO: 404 - protein, 15 aa, Artificial sequence
  Synthetic peptide
DLCFSNVYADSFVVK

SEQ ID NO: 405 - protein, 15 aa, Artificial sequence
  Synthetic peptide
LCFSNVYADSFVVKG

SEQ ID NO: 406 - protein, 15 aa, Artificial sequence
  Synthetic peptide
CFSNVYADSFVVKGD

SEQ ID NO: 407 - protein, 15 aa, Artificial sequence
  Synthetic peptide
FSNVYADSFVVKGDD

SEQ ID NO: 408 - protein, 15 aa, Artificial sequence
  Synthetic peptide
SNVYADSFVVKGDDV

SEQ ID NO: 409 - protein, 15 aa, Artificial sequence
  Synthetic peptide
NVYADSFVVKGDDVR

SEQ ID NO: 410 - protein, 15 aa, Artificial sequence
  Synthetic peptide
VYADSFVVKGDDVRQ

SEQ ID NO: 411 - protein, 15 aa, Artificial sequence
  Synthetic peptide
YADSFVVKGDDVRQI

SEQ ID NO: 412 - protein, 15 aa, Artificial sequence
  Synthetic peptide
ADSFVVKGDDVRQIA

SEQ ID NO: 413 - protein, 15 aa, Artificial sequence
  Synthetic peptide
DSFVVKGDDVRQIAP

SEQ ID NO: 414 - protein, 15 aa, Artificial sequence
  Synthetic peptide
SFVVKGDDVRQIAPG

SEQ ID NO: 415 - protein, 15 aa, Artificial sequence
  Synthetic peptide
FVVKGDDVRQIAPGQ

SEQ ID NO: 416 - protein, 15 aa, Artificial sequence
  Synthetic peptide
VVKGDDVRQIAPGQT

SEQ ID NO: 417 - protein, 15 aa, Artificial sequence
  Synthetic peptide
VKGDDVRQIAPGQTG

SEQ ID NO: 418 - protein, 15 aa, Artificial sequence
  Synthetic peptide
KGDDVRQIAPGQTGV

SEQ ID NO: 419 - protein, 15 aa, Artificial sequence
  Synthetic peptide
GDDVRQIAPGQTGVI

SEQ ID NO: 420 - protein, 15 aa, Artificial sequence
  Synthetic peptide
DDVRQIAPGQTGVIA

SEQ ID NO: 421 - protein, 15 aa, Artificial sequence
  Synthetic peptide
DVRQIAPGQTGVIAD

SEQ ID NO: 422 - protein, 15 aa, Artificial sequence
  Synthetic peptide
VRQIAPGQTGVIADY

SEQ ID NO: 423 - protein, 15 aa, Artificial sequence
  Synthetic peptide
RQIAPGQTGVIADYN

SEQ ID NO: 424 - protein, 15 aa, Artificial sequence
  Synthetic peptide
QIAPGQTGVIADYNY

SEQ ID NO: 425 - protein, 15 aa, Artificial sequence
  Synthetic peptide
IAPGQTGVIADYNYK

SEQ ID NO: 426 - protein, 15 aa, Artificial sequence
  Synthetic peptide
APGQTGVIADYNYKL

SEQ ID NO: 427 - protein, 15 aa, Artificial sequence
  Synthetic peptide
PGQTGVIADYNYKLP

SEQ ID NO: 428 - protein, 15 aa, Artificial sequence
  Synthetic peptide
GQTGVIADYNYKLPD

SEQ ID NO: 429 - protein, 15 aa, Artificial sequence
  Synthetic peptide
QTGVIADYNYKLPDD

SEQ ID NO: 430 - protein, 15 aa, Artificial sequence
  Synthetic peptide
TGVIADYNYKLPDDF

SEQ ID NO: 431 - protein, 15 aa, Artificial sequence
  Synthetic peptide
GVIADYNYKLPDDFM

SEQ ID NO: 432 - protein, 15 aa, Artificial sequence
  Synthetic peptide
VIADYNYKLPDDFMG

SEQ ID NO: 433 - protein, 15 aa, Artificial sequence
  Synthetic peptide
IADYNYKLPDDFMGC

SEQ ID NO: 434 - protein, 15 aa, Artificial sequence
  Synthetic peptide
ADYNYKLPDDFMGCV

SEQ ID NO: 435 - protein, 15 aa, Artificial sequence
  Synthetic peptide
DYNYKLPDDFMGCVL

SEQ ID NO: 436 - protein, 15 aa, Artificial sequence
  Synthetic peptide
YNYKLPDDFMGCVLA

SEQ ID NO: 437 - protein, 15 aa, Artificial sequence
  Synthetic peptide
NYKLPDDFMGCVLAW

SEQ ID NO: 438 - protein, 15 aa, Artificial sequence
  Synthetic peptide
YKLPDDFMGCVLAWN

SEQ ID NO: 439 - protein, 15 aa, Artificial sequence
  Synthetic peptide
KLPDDFMGCVLAWNT

SEQ ID NO: 440 - protein, 15 aa, Artificial sequence
  Synthetic peptide
LPDDFMGCVLAWNTR

SEQ ID NO: 441 - protein, 15 aa, Artificial sequence
  Synthetic peptide
PDDFMGCVLAWNTRN

SEQ ID NO: 442 - protein, 15 aa, Artificial sequence
  Synthetic peptide
DDFMGCVLAWNTRNI

SEQ ID NO: 443 - protein, 15 aa, Artificial sequence
  Synthetic peptide
DFMGCVLAWNTRNID

SEQ ID NO: 444 - protein, 15 aa, Artificial sequence
  Synthetic peptide
FMGCVLAWNTRNIDA

SEQ ID NO: 445 - protein, 15 aa, Artificial sequence
  Synthetic peptide
MGCVLAWNTRNIDAT

SEQ ID NO: 446 - protein, 15 aa, Artificial sequence
  Synthetic peptide
GCVLAWNTRNIDATS

SEQ ID NO: 447 - protein, 15 aa, Artificial sequence
  Synthetic peptide
CVLAWNTRNIDATST

SEQ ID NO: 448 - protein, 15 aa, Artificial sequence
  Synthetic peptide
VLAWNTRNIDATSTG

SEQ ID NO: 449 - protein, 15 aa, Artificial sequence
  Synthetic peptide
LAWNTRNIDATSTGN

SEQ ID NO: 450 - protein, 15 aa, Artificial sequence
  Synthetic peptide
AWNTRNIDATSTGNY

SEQ ID NO: 451 - protein, 15 aa, Artificial sequence
  Synthetic peptide
WNTRNIDATSTGNYN

SEQ ID NO: 452 - protein, 15 aa, Artificial sequence
  Synthetic peptide
NTRNIDATSTGNYNY

SEQ ID NO: 453 - protein, 15 aa, Artificial sequence
  Synthetic peptide
TRNIDATSTGNYNYK

SEQ ID NO: 454 - protein, 15 aa, Artificial sequence
  Synthetic peptide
RNIDATSTGNYNYKY

SEQ ID NO: 455 - protein, 15 aa, Artificial sequence
  Synthetic peptide
NIDATSTGNYNYKYR

SEQ ID NO: 456 - protein, 15 aa, Artificial sequence
  Synthetic peptide
IDATSTGNYNYKYRY

SEQ ID NO: 457 - protein, 15 aa, Artificial sequence
  Synthetic peptide
DATSTGNYNYKYRYL

SEQ ID NO: 458 - protein, 15 aa, Artificial sequence
  Synthetic peptide
ATSTGNYNYKYRYLR

SEQ ID NO: 459 - protein, 15 aa, Artificial sequence
  Synthetic peptide
TSTGNYNYKYRYLRH

SEQ ID NO: 460 - protein, 15 aa, Artificial sequence
  Synthetic peptide
STGNYNYKYRYLRHG

SEQ ID NO: 461 - protein, 15 aa, Artificial sequence
  Synthetic peptide
TGNYNYKYRYLRHGK

SEQ ID NO: 462 - protein, 15 aa, Artificial sequence
  Synthetic peptide
GNYNYKYRYLRHGKL

SEQ ID NO: 463 - protein, 15 aa, Artificial sequence
  Synthetic peptide
NYNYKYRYLRHGKLR

SEQ ID NO: 464 - protein, 15 aa, Artificial sequence
  Synthetic peptide
YNYKYRYLRHGKLRP

SEQ ID NO: 465 - protein, 15 aa, Artificial sequence
  Synthetic peptide
NYKYRYLRHGKLRPF

SEQ ID NO: 466 - protein, 15 aa, Artificial sequence
  Synthetic peptide
YKYRYLRHGKLRPFE

SEQ ID NO: 467 - protein, 15 aa, Artificial sequence
  Synthetic peptide
KYRYLRHGKLRPFER

SEQ ID NO: 468 - protein, 15 aa, Artificial sequence
  Synthetic peptide
YRYLRHGKLRPFERD

SEQ ID NO: 469 - protein, 15 aa, Artificial sequence
  Synthetic peptide
RYLRHGKLRPFERDI

SEQ ID NO: 470 - protein, 15 aa, Artificial sequence
  Synthetic peptide
YLRHGKLRPFERDIS

SEQ ID NO: 471 - protein, 15 aa, Artificial sequence
  Synthetic peptide
LRHGKLRPFERDISN

SEQ ID NO: 472 - protein, 15 aa, Artificial sequence
  Synthetic peptide
RHGKLRPFERDISNV

SEQ ID NO: 473 - protein, 15 aa, Artificial sequence
  Synthetic peptide
HGKLRPFERDISNVP

SEQ ID NO: 474 - protein, 15 aa, Artificial sequence
  Synthetic peptide
GKLRPFERDISNVPF

SEQ ID NO: 475 - protein, 15 aa, Artificial sequence
  Synthetic peptide
KLRPFERDISNVPFS

SEQ ID NO: 476 - protein, 15 aa, Artificial sequence
  Synthetic peptide
LRPFERDISNVPFSP

SEQ ID NO: 477 - protein, 15 aa, Artificial sequence
  Synthetic peptide
RPFERDISNVPFSPD

SEQ ID NO: 478 - protein, 15 aa, Artificial sequence
  Synthetic peptide
PFERDISNVPFSPDG

SEQ ID NO: 479 - protein, 15 aa, Artificial sequence
  Synthetic peptide
FERDISNVPFSPDGK

SEQ ID NO: 480 - protein, 15 aa, Artificial sequence
  Synthetic peptide
ERDISNVPFSPDGKP

SEQ ID NO: 481 - protein, 15 aa, Artificial sequence
  Synthetic peptide
RDISNVPFSPDGKPC

SEQ ID NO: 482 - protein, 15 aa, Artificial sequence
  Synthetic peptide
DISNVPFSPDGKPCT

SEQ ID NO: 483 - protein, 15 aa, Artificial sequence
  Synthetic peptide
ISNVPFSPDGKPCTP

SEQ ID NO: 484 - protein, 15 aa, Artificial sequence
  Synthetic peptide
SNVPFSPDGKPCTPP

SEQ ID NO: 485 - protein, 15 aa, Artificial sequence
  Synthetic peptide
NVPFSPDGKPCTPPA

SEQ ID NO: 486 - protein, 15 aa, Artificial sequence
  Synthetic peptide
VPFSPDGKPCTPPAL

SEQ ID NO: 487 - protein, 15 aa, Artificial sequence
  Synthetic peptide
PFSPDGKPCTPPALN

SEQ ID NO: 488 - protein, 15 aa, Artificial sequence
  Synthetic peptide
FSPDGKPCTPPALNC

SEQ ID NO: 489 - protein, 15 aa, Artificial sequence
  Synthetic peptide
SPDGKPCTPPALNCY

SEQ ID NO: 490 - protein, 15 aa, Artificial sequence
  Synthetic peptide
PDGKPCTPPALNCYW

SEQ ID NO: 491 - protein, 15 aa, Artificial sequence
  Synthetic peptide
DGKPCTPPALNCYWP

SEQ ID NO: 492 - protein, 15 aa, Artificial sequence
  Synthetic peptide
GKPCTPPALNCYWPL

SEQ ID NO: 493 - protein, 15 aa, Artificial sequence
  Synthetic peptide
KPCTPPALNCYWPLN

SEQ ID NO: 494 - protein, 15 aa, Artificial sequence
  Synthetic peptide
PCTPPALNCYWPLND

SEQ ID NO: 495 - protein, 15 aa, Artificial sequence
  Synthetic peptide
CTPPALNCYWPLNDY

SEQ ID NO: 496 - protein, 15 aa, Artificial sequence
  Synthetic peptide
TPPALNCYWPLNDYG

SEQ ID NO: 497 - protein, 15 aa, Artificial sequence
  Synthetic peptide
PPALNCYWPLNDYGF

SEQ ID NO: 498 - protein, 15 aa, Artificial sequence
  Synthetic peptide
PALNCYWPLNDYGFY

SEQ ID NO: 499 - protein, 15 aa, Artificial sequence
  Synthetic peptide
ALNCYWPLNDYGFYT

SEQ ID NO: 500 - protein, 15 aa, Artificial sequence
  Synthetic peptide
LNCYWPLNDYGFYTT

SEQ ID NO: 501 - protein, 15 aa, Artificial sequence
  Synthetic peptide
NCYWPLNDYGFYTTT

SEQ ID NO: 502 - protein, 15 aa, Artificial sequence
  Synthetic peptide
CYWPLNDYGFYTTTG

SEQ ID NO: 503 - protein, 15 aa, Artificial sequence
  Synthetic peptide
YWPLNDYGFYTTTGI

SEQ ID NO: 504 - protein, 15 aa, Artificial sequence
  Synthetic peptide
WPLNDYGFYTTTGIG

SEQ ID NO: 505 - protein, 15 aa, Artificial sequence
  Synthetic peptide
PLNDYGFYTTTGIGY

SEQ ID NO: 506 - protein, 15 aa, Artificial sequence
  Synthetic peptide
LNDYGFYTTTGIGYQ

SEQ ID NO: 507 - protein, 15 aa, Artificial sequence
  Synthetic peptide
NDYGFYTTTGIGYQP

SEQ ID NO: 508 - protein, 15 aa, Artificial sequence
  Synthetic peptide
DYGFYTTTGIGYQPY

SEQ ID NO: 509 - protein, 15 aa, Artificial sequence
  Synthetic peptide
YGFYTTTGIGYQPYR

SEQ ID NO: 510 - protein, 15 aa, Artificial sequence
  Synthetic peptide
GFYTTTGIGYQPYRV

SEQ ID NO: 511 - protein, 15 aa, Artificial sequence
  Synthetic peptide
FYTTTGIGYQPYRVV

SEQ ID NO: 512 - protein, 15 aa, Artificial sequence
  Synthetic peptide
YTTTGIGYQPYRVVV

SEQ ID NO: 513 - protein, 15 aa, Artificial sequence
  Synthetic peptide
TTTGIGYQPYRVVVL

SEQ ID NO: 514 - protein, 15 aa, Artificial sequence
  Synthetic peptide
TTGIGYQPYRVVVLS

SEQ ID NO: 515 - protein, 15 aa, Artificial sequence
  Synthetic peptide
TGIGYQPYRVVVLSF

SEQ ID NO: 516 - protein, 15 aa, Artificial sequence
  Synthetic peptide
GIGYQPYRVVVLSFE

SEQ ID NO: 517 - protein, 15 aa, Artificial sequence
  Synthetic peptide
IGYQPYRVVVLSFEL

SEQ ID NO: 518 - protein, 15 aa, Artificial sequence
  Synthetic peptide
GYQPYRVVVLSFELL

SEQ ID NO: 519 - protein, 15 aa, Artificial sequence
  Synthetic peptide
YQPYRVVVLSFELLN

SEQ ID NO: 520 - protein, 15 aa, Artificial sequence
  Synthetic peptide
QPYRVVVLSFELLNA

SEQ ID NO: 521 - protein, 15 aa, Artificial sequence
  Synthetic peptide
PYRVVVLSFELLNAP

SEQ ID NO: 522 - protein, 15 aa, Artificial sequence
  Synthetic peptide
YRVVVLSFELLNAPA

SEQ ID NO: 523 - protein, 15 aa, Artificial sequence
  Synthetic peptide
RVVVLSFELLNAPAT

SEQ ID NO: 524 - protein, 15 aa, Artificial sequence
  Synthetic peptide
VVVLSFELLNAPATV

SEQ ID NO: 525 - protein, 15 aa, Artificial sequence
  Synthetic peptide
VVLSFELLNAPATVC

SEQ ID NO: 526 - protein, 15 aa, Artificial sequence
  Synthetic peptide
VLSFELLNAPATVCG

SEQ ID NO: 527 - protein, 15 aa, Artificial sequence
  Synthetic peptide
LSFELLNAPATVCGP

SEQ ID NO: 528 - protein, 15 aa, Artificial sequence
  Synthetic peptide
SFELLNAPATVCGPK

SEQ ID NO: 529 - protein, 15 aa, Artificial sequence
  Synthetic peptide
FELLNAPATVCGPKL

SEQ ID NO: 530 - protein, 15 aa, Artificial sequence
  Synthetic peptide
ELLNAPATVCGPKLS

SEQ ID NO: 531 - protein, 15 aa, Artificial sequence
  Synthetic peptide
LLNAPATVCGPKLST

SEQ ID NO: 532 - protein, 15 aa, Artificial sequence
  Synthetic peptide
LNAPATVCGPKLSTD

SEQ ID NO: 533 - protein, 15 aa, Artificial sequence
  Synthetic peptide
NAPATVCGPKLSTDL

SEQ ID NO: 534 - protein, 15 aa, Artificial sequence
  Synthetic peptide
APATVCGPKLSTDLI

SEQ ID NO: 535 - protein, 15 aa, Artificial sequence
  Synthetic peptide
PATVCGPKLSTDLIK

SEQ ID NO: 536 - protein, 15 aa, Artificial sequence
  Synthetic peptide
ATVCGPKLSTDLIKN

SEQ ID NO: 537 - protein, 15 aa, Artificial sequence
  Synthetic peptide
TVCGPKLSTDLIKNQ

SEQ ID NO: 538 - protein, 15 aa, Artificial sequence
  Synthetic peptide
VCGPKLSTDLIKNQC

SEQ ID NO: 539 - protein, 15 aa, Artificial sequence
  Synthetic peptide
CGPKLSTDLIKNQCV

SEQ ID NO: 540 - protein, 15 aa, Artificial sequence
  Synthetic peptide
GPKLSTDLIKNQCVN

SEQ ID NO: 541 - protein, 15 aa, Artificial sequence
  Synthetic peptide
PKLSTDLIKNQCVNF

SEQ ID NO: 542 - protein, 15 aa, Artificial sequence
  Synthetic peptide
KLSTDLIKNQCVNFN

SEQ ID NO: 543 - protein, 15 aa, Artificial sequence
  Synthetic peptide
LSTDLIKNQCVNFNF

SEQ ID NO: 544 - protein, 15 aa, Artificial sequence
  Synthetic peptide
STDLIKNQCVNFNFN

SEQ ID NO: 545 - protein, 15 aa, Artificial sequence
  Synthetic peptide
TDLIKNQCVNFNFNG

SEQ ID NO: 546 - protein, 15 aa, Artificial sequence
  Synthetic peptide
DLIKNQCVNFNFNGL

SEQ ID NO: 547 - protein, 15 aa, Artificial sequence
  Synthetic peptide
LIKNQCVNFNFNGLT

SEQ ID NO: 548 - protein, 15 aa, Artificial sequence
  Synthetic peptide
IKNQCVNFNFNGLTG

SEQ ID NO: 549 - protein, 15 aa, Artificial sequence
  Synthetic peptide
KNQCVNFNFNGLTGT

SEQ ID NO: 550 - protein, 15 aa, Artificial sequence
  Synthetic peptide
NQCVNFNFNGLTGTG

SEQ ID NO: 551 - protein, 15 aa, Artificial sequence
  Synthetic peptide
QCVNFNFNGLTGTGV

SEQ ID NO: 552 - protein, 15 aa, Artificial sequence
  Synthetic peptide
CVNFNFNGLTGTGVL

SEQ ID NO: 553 - protein, 15 aa, Artificial sequence
  Synthetic peptide
VNFNFNGLTGTGVLT

SEQ ID NO: 554 - protein, 15 aa, Artificial sequence
  Synthetic peptide
NFNFNGLTGTGVLTP

SEQ ID NO: 555 - protein, 15 aa, Artificial sequence
  Synthetic peptide
FNFNGLTGTGVLTPS

SEQ ID NO: 556 - protein, 15 aa, Artificial sequence
  Synthetic peptide
NFNGLTGTGVLTPSS

SEQ ID NO: 557 - protein, 15 aa, Artificial sequence
  Synthetic peptide
FNGLTGTGVLTPSSK

SEQ ID NO: 558 - protein, 15 aa, Artificial sequence
  Synthetic peptide
NGLTGTGVLTPSSKR

SEQ ID NO: 559 - protein, 15 aa, Artificial sequence
  Synthetic peptide
GLTGTGVLTPSSKRF

SEQ ID NO: 560 - protein, 15 aa, Artificial sequence
  Synthetic peptide
LTGTGVLTPSSKRFQ

SEQ ID NO: 561 - protein, 15 aa, Artificial sequence
  Synthetic peptide
TGTGVLTPSSKRFQP

SEQ ID NO: 562 - protein, 15 aa, Artificial sequence
  Synthetic peptide
GTGVLTPSSKRFQPF

SEQ ID NO: 563 - protein, 15 aa, Artificial sequence
  Synthetic peptide
TGVLTPSSKRFQPFQ

SEQ ID NO: 564 - protein, 15 aa, Artificial sequence
  Synthetic peptide
GVLTPSSKRFQPFQQ

SEQ ID NO: 565 - protein, 15 aa, Artificial sequence
  Synthetic peptide
VLTPSSKRFQPFQQF

SEQ ID NO: 566 - protein, 15 aa, Artificial sequence
  Synthetic peptide
LTPSSKRFQPFQQFG

SEQ ID NO: 567 - protein, 15 aa, Artificial sequence
  Synthetic peptide
TPSSKRFQPFQQFGR

SEQ ID NO: 568 - protein, 15 aa, Artificial sequence
  Synthetic peptide
PSSKRFQPFQQFGRD

SEQ ID NO: 569 - protein, 15 aa, Artificial sequence
  Synthetic peptide
SSKRFQPFQQFGRDV

SEQ ID NO: 570 - protein, 15 aa, Artificial sequence
  Synthetic peptide
SKRFQPFQQFGRDVS

SEQ ID NO: 571 - protein, 15 aa, Artificial sequence
  Synthetic peptide
KRFQPFQQFGRDVSD

SEQ ID NO: 572 - protein, 15 aa, Artificial sequence
  Synthetic peptide
RFQPFQQFGRDVSDF

SEQ ID NO: 573 - protein, 15 aa, Artificial sequence
  Synthetic peptide
FQPFQQFGRDVSDFT

SEQ ID NO: 574 - protein, 15 aa, Artificial sequence
  Synthetic peptide
QPFQQFGRDVSDFTD

SEQ ID NO: 575 - protein, 15 aa, Artificial sequence
  Synthetic peptide
PFQQFGRDVSDFTDS

SEQ ID NO: 576 - protein, 15 aa, Artificial sequence
  Synthetic peptide
FQQFGRDVSDFTDSV

SEQ ID NO: 577 - protein, 15 aa, Artificial sequence
  Synthetic peptide
QQFGRDVSDFTDSVR

SEQ ID NO: 578 - protein, 15 aa, Artificial sequence
  Synthetic peptide
QFGRDVSDFTDSVRD

SEQ ID NO: 579 - protein, 15 aa, Artificial sequence
  Synthetic peptide
FGRDVSDFTDSVRDP

SEQ ID NO: 580 - protein, 15 aa, Artificial sequence
  Synthetic peptide
GRDVSDFTDSVRDPK

SEQ ID NO: 581 - protein, 15 aa, Artificial sequence
  Synthetic peptide
RDVSDFTDSVRDPKT

SEQ ID NO: 582 - protein, 15 aa, Artificial sequence
  Synthetic peptide
DVSDFTDSVRDPKTS

SEQ ID NO: 583 - protein, 15 aa, Artificial sequence
  Synthetic peptide
VSDFTDSVRDPKTSE

SEQ ID NO: 584 - protein, 15 aa, Artificial sequence
  Synthetic peptide
SDFTDSVRDPKTSEI

SEQ ID NO: 585 - protein, 15 aa, Artificial sequence
  Synthetic peptide
DFTDSVRDPKTSEIL

SEQ ID NO: 586 - protein, 15 aa, Artificial sequence
  Synthetic peptide
FTDSVRDPKTSEILD

SEQ ID NO: 587 - protein, 15 aa, Artificial sequence
  Synthetic peptide
TDSVRDPKTSEILDI

SEQ ID NO: 588 - protein, 15 aa, Artificial sequence
  Synthetic peptide
DSVRDPKTSEILDIS

SEQ ID NO: 589 - protein, 15 aa, Artificial sequence
  Synthetic peptide
SVRDPKTSEILDISP

SEQ ID NO: 590 - protein, 15 aa, Artificial sequence
  Synthetic peptide
VRDPKTSEILDISPC

SEQ ID NO: 591 - protein, 15 aa, Artificial sequence
  Synthetic peptide
RDPKTSEILDISPCS

SEQ ID NO: 592 - protein, 15 aa, Artificial sequence
  Synthetic peptide
DPKTSEILDISPCSF

SEQ ID NO: 593 - protein, 15 aa, Artificial sequence
  Synthetic peptide
PKTSEILDISPCSFG

SEQ ID NO: 594 - protein, 15 aa, Artificial sequence
  Synthetic peptide
KTSEILDISPCSFGG

SEQ ID NO: 595 - protein, 15 aa, Artificial sequence
  Synthetic peptide
TSEILDISPCSFGGV

SEQ ID NO: 596 - protein, 15 aa, Artificial sequence
  Synthetic peptide
SEILDISPCSFGGVS

SEQ ID NO: 597 - protein, 15 aa, Artificial sequence
  Synthetic peptide
EILDISPCSFGGVSV

SEQ ID NO: 598 - protein, 15 aa, Artificial sequence
  Synthetic peptide
ILDISPCSFGGVSVI

SEQ ID NO: 599 - protein, 15 aa, Artificial sequence
  Synthetic peptide
LDISPCSFGGVSVIT

SEQ ID NO: 600 - protein, 15 aa, Artificial sequence
  Synthetic peptide
DISPCSFGGVSVITP

SEQ ID NO: 601 - protein, 15 aa, Artificial sequence
  Synthetic peptide
ISPCSFGGVSVITPG

SEQ ID NO: 602 - protein, 15 aa, Artificial sequence
  Synthetic peptide
SPCSFGGVSVITPGT

SEQ ID NO: 603 - protein, 15 aa, Artificial sequence
  Synthetic peptide
PCSFGGVSVITPGTN

SEQ ID NO: 604 - protein, 15 aa, Artificial sequence
  Synthetic peptide
CSFGGVSVITPGTNA

SEQ ID NO: 605 - protein, 15 aa, Artificial sequence
  Synthetic peptide
SFGGVSVITPGTNAS

SEQ ID NO: 606 - protein, 15 aa, Artificial sequence
  Synthetic peptide
FGGVSVITPGTNASS

SEQ ID NO: 607 - protein, 15 aa, Artificial sequence
  Synthetic peptide
GGVSVITPGTNASSE

SEQ ID NO: 608 - protein, 15 aa, Artificial sequence
  Synthetic peptide
GVSVITPGTNASSEV

SEQ ID NO: 609 - protein, 15 aa, Artificial sequence
  Synthetic peptide
VSVITPGTNASSEVA

SEQ ID NO: 610 - protein, 15 aa, Artificial sequence
  Synthetic peptide
SVITPGTNASSEVAV

SEQ ID NO: 611 - protein, 15 aa, Artificial sequence
  Synthetic peptide
VITPGTNASSEVAVL

SEQ ID NO: 612 - protein, 15 aa, Artificial sequence
  Synthetic peptide
ITPGTNASSEVAVLY

SEQ ID NO: 613 - protein, 15 aa, Artificial sequence
  Synthetic peptide
TPGTNASSEVAVLYQ

SEQ ID NO: 614 - protein, 15 aa, Artificial sequence
  Synthetic peptide
PGTNASSEVAVLYQD

SEQ ID NO: 615 - protein, 15 aa, Artificial sequence
  Synthetic peptide
GTNASSEVAVLYQDV

SEQ ID NO: 616 - protein, 15 aa, Artificial sequence
  Synthetic peptide
TNASSEVAVLYQDVN

SEQ ID NO: 617 - protein, 15 aa, Artificial sequence
  Synthetic peptide
NASSEVAVLYQDVNC

SEQ ID NO: 618 - protein, 15 aa, Artificial sequence
  Synthetic peptide
ASSEVAVLYQDVNCT

SEQ ID NO: 619 - protein, 15 aa, Artificial sequence
  Synthetic peptide
SSEVAVLYQDVNCTD

SEQ ID NO: 620 - protein, 15 aa, Artificial sequence
  Synthetic peptide
SEVAVLYQDVNCTDV

SEQ ID NO: 621 - protein, 15 aa, Artificial sequence
  Synthetic peptide
EVAVLYQDVNCTDVS

SEQ ID NO: 622 - protein, 15 aa, Artificial sequence
  Synthetic peptide
VAVLYQDVNCTDVST

SEQ ID NO: 623 - protein, 15 aa, Artificial sequence
  Synthetic peptide
AVLYQDVNCTDVSTA

SEQ ID NO: 624 - protein, 15 aa, Artificial sequence
  Synthetic peptide
VLYQDVNCTDVSTAI

SEQ ID NO: 625 - protein, 15 aa, Artificial sequence
  Synthetic peptide
LYQDVNCTDVSTAIH

SEQ ID NO: 626 - protein, 15 aa, Artificial sequence
  Synthetic peptide
YQDVNCTDVSTAIHA

SEQ ID NO: 627 - protein, 15 aa, Artificial sequence
  Synthetic peptide
QDVNCTDVSTAIHAD

SEQ ID NO: 628 - protein, 15 aa, Artificial sequence
  Synthetic peptide
DVNCTDVSTAIHADQ

SEQ ID NO: 629 - protein, 15 aa, Artificial sequence
  Synthetic peptide
VNCTDVSTAIHADQL

SEQ ID NO: 630 - protein, 15 aa, Artificial sequence
  Synthetic peptide
NCTDVSTAIHADQLT

SEQ ID NO: 631 - protein, 15 aa, Artificial sequence
  Synthetic peptide
CTDVSTAIHADQLTP

SEQ ID NO: 632 - protein, 15 aa, Artificial sequence
  Synthetic peptide
TDVSTAIHADQLTPA

SEQ ID NO: 633 - protein, 15 aa, Artificial sequence
  Synthetic peptide
DVSTAIHADQLTPAW

SEQ ID NO: 634 - protein, 15 aa, Artificial sequence
  Synthetic peptide
VSTAIHADQLTPAWR

SEQ ID NO: 635 - protein, 15 aa, Artificial sequence
  Synthetic peptide
STAIHADQLTPAWRI

SEQ ID NO: 636 - protein, 15 aa, Artificial sequence
  Synthetic peptide
TAIHADQLTPAWRIY

SEQ ID NO: 637 - protein, 15 aa, Artificial sequence
  Synthetic peptide
AIHADQLTPAWRIYS

SEQ ID NO: 638 - protein, 15 aa, Artificial sequence
  Synthetic peptide
IHADQLTPAWRIYST

SEQ ID NO: 639 - protein, 15 aa, Artificial sequence
  Synthetic peptide
HADQLTPAWRIYSTG

SEQ ID NO: 640 - protein, 15 aa, Artificial sequence
  Synthetic peptide
ADQLTPAWRIYSTGN

SEQ ID NO: 641 - protein, 15 aa, Artificial sequence
  Synthetic peptide
DQLTPAWRIYSTGNN

SEQ ID NO: 642 - protein, 15 aa, Artificial sequence
  Synthetic peptide
QLTPAWRIYSTGNNV

SEQ ID NO: 643 - protein, 15 aa, Artificial sequence
  Synthetic peptide
LTPAWRIYSTGNNVF

SEQ ID NO: 644 - protein, 15 aa, Artificial sequence
  Synthetic peptide
TPAWRIYSTGNNVFQ

SEQ ID NO: 645 - protein, 15 aa, Artificial sequence
  Synthetic peptide
PAWRIYSTGNNVFQT

SEQ ID NO: 646 - protein, 15 aa, Artificial sequence
  Synthetic peptide
AWRIYSTGNNVFQTQ

SEQ ID NO: 647 - protein, 15 aa, Artificial sequence
  Synthetic peptide
WRIYSTGNNVFQTQA

SEQ ID NO: 648 - protein, 15 aa, Artificial sequence
  Synthetic peptide
RIYSTGNNVFQTQAG

SEQ ID NO: 649 - protein, 15 aa, Artificial sequence
  Synthetic peptide
IYSTGNNVFQTQAGC

SEQ ID NO: 650 - protein, 15 aa, Artificial sequence
  Synthetic peptide
YSTGNNVFQTQAGCL

SEQ ID NO: 651 - protein, 15 aa, Artificial sequence
  Synthetic peptide
STGNNVFQTQAGCLI

SEQ ID NO: 652 - protein, 15 aa, Artificial sequence
  Synthetic peptide
TGNNVFQTQAGCLIG

SEQ ID NO: 653 - protein, 15 aa, Artificial sequence
  Synthetic peptide
GNNVFQTQAGCLIGA

SEQ ID NO: 654 - protein, 15 aa, Artificial sequence
  Synthetic peptide
NNVFQTQAGCLIGAE

SEQ ID NO: 655 - protein, 15 aa, Artificial sequence
  Synthetic peptide
NVFQTQAGCLIGAEH

SEQ ID NO: 656 - protein, 15 aa, Artificial sequence
  Synthetic peptide
VFQTQAGCLIGAEHV

SEQ ID NO: 657 - protein, 15 aa, Artificial sequence
  Synthetic peptide
FQTQAGCLIGAEHVD

SEQ ID NO: 658 - protein, 15 aa, Artificial sequence
  Synthetic peptide
QTQAGCLIGAEHVDT

SEQ ID NO: 659 - protein, 15 aa, Artificial sequence
  Synthetic peptide
TQAGCLIGAEHVDTS

SEQ ID NO: 660 - protein, 15 aa, Artificial sequence
  Synthetic peptide
QAGCLIGAEHVDTSY

SEQ ID NO: 661 - protein, 15 aa, Artificial sequence
  Synthetic peptide
AGCLIGAEHVDTSYE

SEQ ID NO: 662 - protein, 15 aa, Artificial sequence
  Synthetic peptide
GCLIGAEHVDTSYEC

SEQ ID NO: 663 - protein, 15 aa, Artificial sequence
  Synthetic peptide
CLIGAEHVDTSYECD

SEQ ID NO: 664 - protein, 15 aa, Artificial sequence
  Synthetic peptide
LIGAEHVDTSYECDI

SEQ ID NO: 665 - protein, 15 aa, Artificial sequence
  Synthetic peptide
IGAEHVDTSYECDIP

SEQ ID NO: 666 - protein, 15 aa, Artificial sequence
  Synthetic peptide
GAEHVDTSYECDIPI

SEQ ID NO: 667 - protein, 15 aa, Artificial sequence
  Synthetic peptide
AEHVDTSYECDIPIG

SEQ ID NO: 668 - protein, 15 aa, Artificial sequence
  Synthetic peptide
EHVDTSYECDIPIGA

SEQ ID NO: 669 - protein, 15 aa, Artificial sequence
  Synthetic peptide
HVDTSYECDIPIGAG

SEQ ID NO: 670 - protein, 15 aa, Artificial sequence
  Synthetic peptide
VDTSYECDIPIGAGI

SEQ ID NO: 671 - protein, 15 aa, Artificial sequence
  Synthetic peptide
DTSYECDIPIGAGIC

SEQ ID NO: 672 - protein, 15 aa, Artificial sequence
  Synthetic peptide
TSYECDIPIGAGICA

SEQ ID NO: 673 - protein, 15 aa, Artificial sequence
  Synthetic peptide
SYECDIPIGAGICAS

SEQ ID NO: 674 - protein, 15 aa, Artificial sequence
  Synthetic peptide
YECDIPIGAGICASY

SEQ ID NO: 675 - protein, 15 aa, Artificial sequence
  Synthetic peptide
ECDIPIGAGICASYH

SEQ ID NO: 676 - protein, 15 aa, Artificial sequence
  Synthetic peptide
CDIPIGAGICASYHT

SEQ ID NO: 677 - protein, 15 aa, Artificial sequence
  Synthetic peptide
DIPIGAGICASYHTV

SEQ ID NO: 678 - protein, 15 aa, Artificial sequence
  Synthetic peptide
IPIGAGICASYHTVS

SEQ ID NO: 679 - protein, 15 aa, Artificial sequence
  Synthetic peptide
PIGAGICASYHTVSL

SEQ ID NO: 680 - protein, 15 aa, Artificial sequence
  Synthetic peptide
IGAGICASYHTVSLL

SEQ ID NO: 681 - protein, 15 aa, Artificial sequence
  Synthetic peptide
GAGICASYHTVSLLR

SEQ ID NO: 682 - protein, 15 aa, Artificial sequence
  Synthetic peptide
AGICASYHTVSLLRS

SEQ ID NO: 683 - protein, 15 aa, Artificial sequence
  Synthetic peptide
GICASYHTVSLLRST

SEQ ID NO: 684 - protein, 15 aa, Artificial sequence
  Synthetic peptide
ICASYHTVSLLRSTS

SEQ ID NO: 685 - protein, 15 aa, Artificial sequence
  Synthetic peptide
CASYHTVSLLRSTSQ

SEQ ID NO: 686 - protein, 15 aa, Artificial sequence
  Synthetic peptide
ASYHTVSLLRSTSQK

SEQ ID NO: 687 - protein, 15 aa, Artificial sequence
  Synthetic peptide
SYHTVSLLRSTSQKS

SEQ ID NO: 688 - protein, 15 aa, Artificial sequence
  Synthetic peptide
YHTVSLLRSTSQKSI

SEQ ID NO: 689 - protein, 15 aa, Artificial sequence
  Synthetic peptide
HTVSLLRSTSQKSIV

SEQ ID NO: 690 - protein, 15 aa, Artificial sequence
  Synthetic peptide
TVSLLRSTSQKSIVA

SEQ ID NO: 691 - protein, 15 aa, Artificial sequence
  Synthetic peptide
VSLLRSTSQKSIVAY

SEQ ID NO: 692 - protein, 15 aa, Artificial sequence
  Synthetic peptide
SLLRSTSQKSIVAYT

SEQ ID NO: 693 - protein, 15 aa, Artificial sequence
  Synthetic peptide
LLRSTSQKSIVAYTM

SEQ ID NO: 694 - protein, 15 aa, Artificial sequence
  Synthetic peptide
LRSTSQKSIVAYTMS

SEQ ID NO: 695 - protein, 15 aa, Artificial sequence
  Synthetic peptide
RSTSQKSIVAYTMSL

SEQ ID NO: 696 - protein, 15 aa, Artificial sequence
  Synthetic peptide
STSQKSIVAYTMSLG

SEQ ID NO: 697 - protein, 15 aa, Artificial sequence
  Synthetic peptide
TSQKSIVAYTMSLGA

SEQ ID NO: 698 - protein, 15 aa, Artificial sequence
  Synthetic peptide
SQKSIVAYTMSLGAD

SEQ ID NO: 699 - protein, 15 aa, Artificial sequence
  Synthetic peptide
QKSIVAYTMSLGADS

SEQ ID NO: 700 - protein, 15 aa, Artificial sequence
  Synthetic peptide
KSIVAYTMSLGADSS

SEQ ID NO: 701 - protein, 15 aa, Artificial sequence
  Synthetic peptide
SIVAYTMSLGADSSI

SEQ ID NO: 702 - protein, 15 aa, Artificial sequence
  Synthetic peptide
IVAYTMSLGADSSIA

SEQ ID NO: 703 - protein, 15 aa, Artificial sequence
  Synthetic peptide
VAYTMSLGADSSIAY

SEQ ID NO: 704 - protein, 15 aa, Artificial sequence
  Synthetic peptide
AYTMSLGADSSIAYS

SEQ ID NO: 705 - protein, 15 aa, Artificial sequence
  Synthetic peptide
YTMSLGADSSIAYSN

SEQ ID NO: 706 - protein, 15 aa, Artificial sequence
  Synthetic peptide
TMSLGADSSIAYSNN

SEQ ID NO: 707 - protein, 15 aa, Artificial sequence
  Synthetic peptide
MSLGADSSIAYSNNT

SEQ ID NO: 708 - protein, 15 aa, Artificial sequence
  Synthetic peptide
SLGADSSIAYSNNTI

SEQ ID NO: 709 - protein, 15 aa, Artificial sequence
  Synthetic peptide
LGADSSIAYSNNTIA

SEQ ID NO: 710 - protein, 15 aa, Artificial sequence
  Synthetic peptide
GADSSIAYSNNTIAI

SEQ ID NO: 711 - protein, 15 aa, Artificial sequence
  Synthetic peptide
ADSSIAYSNNTIAIP

SEQ ID NO: 712 - protein, 15 aa, Artificial sequence
  Synthetic peptide
DSSIAYSNNTIAIPT

SEQ ID NO: 713 - protein, 15 aa, Artificial sequence
  Synthetic peptide
SSIAYSNNTIAIPTN

SEQ ID NO: 714 - protein, 15 aa, Artificial sequence
  Synthetic peptide
SIAYSNNTIAIPTNF

SEQ ID NO: 715 - protein, 15 aa, Artificial sequence
  Synthetic peptide
IAYSNNTIAIPTNFS

SEQ ID NO: 716 - protein, 15 aa, Artificial sequence
  Synthetic peptide
AYSNNTIAIPTNFSI

SEQ ID NO: 717 - protein, 15 aa, Artificial sequence
  Synthetic peptide
YSNNTIAIPTNFSIS

SEQ ID NO: 718 - protein, 15 aa, Artificial sequence
  Synthetic peptide
SNNTIAIPTNFSISI

SEQ ID NO: 719 - protein, 15 aa, Artificial sequence
  Synthetic peptide
NNTIAIPTNFSISIT

SEQ ID NO: 720 - protein, 15 aa, Artificial sequence
  Synthetic peptide
NTIAIPTNFSISITT

SEQ ID NO: 721 - protein, 15 aa, Artificial sequence
  Synthetic peptide
TIAIPTNFSISITTE

SEQ ID NO: 722 - protein, 15 aa, Artificial sequence
  Synthetic peptide
IAIPTNFSISITTEV

SEQ ID NO: 723 - protein, 15 aa, Artificial sequence
  Synthetic peptide
AIPTNFSISITTEVM

SEQ ID NO: 724 - protein, 15 aa, Artificial sequence
  Synthetic peptide
IPTNFSISITTEVMP

SEQ ID NO: 725 - protein, 15 aa, Artificial sequence
  Synthetic peptide
PTNFSISITTEVMPV

SEQ ID NO: 726 - protein, 15 aa, Artificial sequence
  Synthetic peptide
TNFSISITTEVMPVS

SEQ ID NO: 727 - protein, 15 aa, Artificial sequence
  Synthetic peptide
NFSISITTEVMPVSM

SEQ ID NO: 728 - protein, 15 aa, Artificial sequence
  Synthetic peptide
FSISITTEVMPVSMA

SEQ ID NO: 729 - protein, 15 aa, Artificial sequence
  Synthetic peptide
SISITTEVMPVSMAK

SEQ ID NO: 730 - protein, 15 aa, Artificial sequence
  Synthetic peptide
ISITTEVMPVSMAKT

SEQ ID NO: 731 - protein, 15 aa, Artificial sequence
  Synthetic peptide
SITTEVMPVSMAKTS

SEQ ID NO: 732 - protein, 15 aa, Artificial sequence
  Synthetic peptide
ITTEVMPVSMAKTSV

SEQ ID NO: 733 - protein, 15 aa, Artificial sequence
  Synthetic peptide
TTEVMPVSMAKTSVD

SEQ ID NO: 734 - protein, 15 aa, Artificial sequence
  Synthetic peptide
TEVMPVSMAKTSVDC

SEQ ID NO: 735 - protein, 15 aa, Artificial sequence
  Synthetic peptide
EVMPVSMAKTSVDCN

SEQ ID NO: 736 - protein, 15 aa, Artificial sequence
  Synthetic peptide
VMPVSMAKTSVDCNM

SEQ ID NO: 737 - protein, 15 aa, Artificial sequence
  Synthetic peptide
MPVSMAKTSVDCNMY

SEQ ID NO: 738 - protein, 15 aa, Artificial sequence
  Synthetic peptide
PVSMAKTSVDCNMYI

SEQ ID NO: 739 - protein, 15 aa, Artificial sequence
  Synthetic peptide
VSMAKTSVDCNMYIC

SEQ ID NO: 740 - protein, 15 aa, Artificial sequence
  Synthetic peptide
SMAKTSVDCNMYICG

SEQ ID NO: 741 - protein, 15 aa, Artificial sequence
  Synthetic peptide
MAKTSVDCNMYICGD

SEQ ID NO: 742 - protein, 15 aa, Artificial sequence
  Synthetic peptide
AKTSVDCNMYICGDS

SEQ ID NO: 743 - protein, 15 aa, Artificial sequence
  Synthetic peptide
KTSVDCNMYICGDST

SEQ ID NO: 744 - protein, 15 aa, Artificial sequence
  Synthetic peptide
TSVDCNMYICGDSTE

SEQ ID NO: 745 - protein, 15 aa, Artificial sequence
  Synthetic peptide
SVDCNMYICGDSTEC

SEQ ID NO: 746 - protein, 15 aa, Artificial sequence
  Synthetic peptide
VDCNMYICGDSTECA

SEQ ID NO: 747 - protein, 15 aa, Artificial sequence
  Synthetic peptide
DCNMYICGDSTECAN

SEQ ID NO: 748 - protein, 15 aa, Artificial sequence
  Synthetic peptide
CNMYICGDSTECANL

SEQ ID NO: 749 - protein, 15 aa, Artificial sequence
  Synthetic peptide
NMYICGDSTECANLL

SEQ ID NO: 750 - protein, 15 aa, Artificial sequence
  Synthetic peptide
MYICGDSTECANLLL

SEQ ID NO: 751 - protein, 15 aa, Artificial sequence
  Synthetic peptide
YICGDSTECANLLLQ

SEQ ID NO: 752 - protein, 15 aa, Artificial sequence
  Synthetic peptide
ICGDSTECANLLLQY

SEQ ID NO: 753 - protein, 15 aa, Artificial sequence
  Synthetic peptide
CGDSTECANLLLQYG

SEQ ID NO: 754 - protein, 15 aa, Artificial sequence
  Synthetic peptide
GDSTECANLLLQYGS

SEQ ID NO: 755 - protein, 15 aa, Artificial sequence
  Synthetic peptide
DSTECANLLLQYGSF

SEQ ID NO: 756 - protein, 15 aa, Artificial sequence
  Synthetic peptide
STECANLLLQYGSFC

SEQ ID NO: 757 - protein, 15 aa, Artificial sequence
  Synthetic peptide
TECANLLLQYGSFCT

SEQ ID NO: 758 - protein, 15 aa, Artificial sequence
  Synthetic peptide
ECANLLLQYGSFCTQ

SEQ ID NO: 759 - protein, 15 aa, Artificial sequence
  Synthetic peptide
CANLLLQYGSFCTQL

SEQ ID NO: 760 - protein, 15 aa, Artificial sequence
  Synthetic peptide
ANLLLQYGSFCTQLN

SEQ ID NO: 761 - protein, 15 aa, Artificial sequence
  Synthetic peptide
NLLLQYGSFCTQLNR

SEQ ID NO: 762 - protein, 15 aa, Artificial sequence
  Synthetic peptide
LLLQYGSFCTQLNRA

SEQ ID NO: 763 - protein, 15 aa, Artificial sequence
  Synthetic peptide
LLQYGSFCTQLNRAL

SEQ ID NO: 764 - protein, 15 aa, Artificial sequence
  Synthetic peptide
LQYGSFCTQLNRALS

SEQ ID NO: 765 - protein, 15 aa, Artificial sequence
  Synthetic peptide
QYGSFCTQLNRALSG

SEQ ID NO: 766 - protein, 15 aa, Artificial sequence
  Synthetic peptide
YGSFCTQLNRALSGI

SEQ ID NO: 767 - protein, 15 aa, Artificial sequence
  Synthetic peptide
GSFCTQLNRALSGIA

SEQ ID NO: 768 - protein, 15 aa, Artificial sequence
  Synthetic peptide
SFCTQLNRALSGIAA

SEQ ID NO: 769 - protein, 15 aa, Artificial sequence
  Synthetic peptide
FCTQLNRALSGIAAE

SEQ ID NO: 770 - protein, 15 aa, Artificial sequence
  Synthetic peptide
CTQLNRALSGIAAEQ

SEQ ID NO: 771 - protein, 15 aa, Artificial sequence
  Synthetic peptide
TQLNRALSGIAAEQD

SEQ ID NO: 772 - protein, 15 aa, Artificial sequence
  Synthetic peptide
QLNRALSGIAAEQDR

SEQ ID NO: 773 - protein, 15 aa, Artificial sequence
  Synthetic peptide
LNRALSGIAAEQDRN

SEQ ID NO: 774 - protein, 15 aa, Artificial sequence
  Synthetic peptide
NRALSGIAAEQDRNT

SEQ ID NO: 775 - protein, 15 aa, Artificial sequence
  Synthetic peptide
RALSGIAAEQDRNTR

SEQ ID NO: 776 - protein, 15 aa, Artificial sequence
  Synthetic peptide
ALSGIAAEQDRNTRE

SEQ ID NO: 777 - protein, 15 aa, Artificial sequence
  Synthetic peptide
LSGIAAEQDRNTREV

SEQ ID NO: 778 - protein, 15 aa, Artificial sequence
  Synthetic peptide
SGIAAEQDRNTREVF

SEQ ID NO: 779 - protein, 15 aa, Artificial sequence
  Synthetic peptide
GIAAEQDRNTREVFA

SEQ ID NO: 780 - protein, 15 aa, Artificial sequence
  Synthetic peptide
IAAEQDRNTREVFAQ

SEQ ID NO: 781 - protein, 15 aa, Artificial sequence
  Synthetic peptide
AAEQDRNTREVFAQV

SEQ ID NO: 782 - protein, 15 aa, Artificial sequence
  Synthetic peptide
AEQDRNTREVFAQVK

SEQ ID NO: 783 - protein, 15 aa, Artificial sequence
  Synthetic peptide
EQDRNTREVFAQVKQ

SEQ ID NO: 784 - protein, 15 aa, Artificial sequence
  Synthetic peptide
QDRNTREVFAQVKQM

SEQ ID NO: 785 - protein, 15 aa, Artificial sequence
  Synthetic peptide
DRNTREVFAQVKQMY

SEQ ID NO: 786 - protein, 15 aa, Artificial sequence
  Synthetic peptide
RNTREVFAQVKQMYK

SEQ ID NO: 787 - protein, 15 aa, Artificial sequence
  Synthetic peptide
NTREVFAQVKQMYKT

SEQ ID NO: 788 - protein, 15 aa, Artificial sequence
  Synthetic peptide
TREVFAQVKQMYKTP

SEQ ID NO: 789 - protein, 15 aa, Artificial sequence
  Synthetic peptide
REVFAQVKQMYKTPT

SEQ ID NO: 790 - protein, 15 aa, Artificial sequence
  Synthetic peptide
EVFAQVKQMYKTPTL

SEQ ID NO: 791 - protein, 15 aa, Artificial sequence
  Synthetic peptide
VFAQVKQMYKTPTLK

SEQ ID NO: 792 - protein, 15 aa, Artificial sequence
  Synthetic peptide
FAQVKQMYKTPTLKY

SEQ ID NO: 793 - protein, 15 aa, Artificial sequence
  Synthetic peptide
AQVKQMYKTPTLKYF

SEQ ID NO: 794 - protein, 15 aa, Artificial sequence
  Synthetic peptide
QVKQMYKTPTLKYFG

SEQ ID NO: 795 - protein, 15 aa, Artificial sequence
  Synthetic peptide
VKQMYKTPTLKYFGG

SEQ ID NO: 796 - protein, 15 aa, Artificial sequence
  Synthetic peptide
KQMYKTPTLKYFGGF

SEQ ID NO: 797 - protein, 15 aa, Artificial sequence
  Synthetic peptide
QMYKTPTLKYFGGFN

SEQ ID NO: 798 - protein, 15 aa, Artificial sequence
  Synthetic peptide
MYKTPTLKYFGGFNF

SEQ ID NO: 799 - protein, 15 aa, Artificial sequence
  Synthetic peptide
YKTPTLKYFGGFNFS

SEQ ID NO: 800 - protein, 15 aa, Artificial sequence
  Synthetic peptide
KTPTLKYFGGFNFSQ

SEQ ID NO: 801 - protein, 15 aa, Artificial sequence
  Synthetic peptide
TPTLKYFGGFNFSQI

SEQ ID NO: 802 - protein, 15 aa, Artificial sequence
  Synthetic peptide
PTLKYFGGFNFSQIL

SEQ ID NO: 803 - protein, 15 aa, Artificial sequence
  Synthetic peptide
TLKYFGGFNFSQILP

SEQ ID NO: 804 - protein, 15 aa, Artificial sequence
  Synthetic peptide
LKYFGGFNFSQILPD

SEQ ID NO: 805 - protein, 15 aa, Artificial sequence
  Synthetic peptide
KYFGGFNFSQILPDP

SEQ ID NO: 806 - protein, 15 aa, Artificial sequence
  Synthetic peptide
YFGGFNFSQILPDPL

SEQ ID NO: 807 - protein, 15 aa, Artificial sequence
  Synthetic peptide
FGGFNFSQILPDPLK

SEQ ID NO: 808 - protein, 15 aa, Artificial sequence
  Synthetic peptide
GGFNFSQILPDPLKP

SEQ ID NO: 809 - protein, 15 aa, Artificial sequence
  Synthetic peptide
GFNFSQILPDPLKPT

SEQ ID NO: 810 - protein, 15 aa, Artificial sequence
  Synthetic peptide
FNFSQILPDPLKPTK

SEQ ID NO: 811 - protein, 15 aa, Artificial sequence
  Synthetic peptide
NFSQILPDPLKPTKR

SEQ ID NO: 812 - protein, 15 aa, Artificial sequence
  Synthetic peptide
FSQILPDPLKPTKRS

SEQ ID NO: 813 - protein, 15 aa, Artificial sequence
  Synthetic peptide
SQILPDPLKPTKRSF

SEQ ID NO: 814 - protein, 15 aa, Artificial sequence
  Synthetic peptide
QILPDPLKPTKRSFI

SEQ ID NO: 815 - protein, 15 aa, Artificial sequence
  Synthetic peptide
ILPDPLKPTKRSFIE

SEQ ID NO: 816 - protein, 15 aa, Artificial sequence
  Synthetic peptide
LPDPLKPTKRSFIED

SEQ ID NO: 817 - protein, 15 aa, Artificial sequence
  Synthetic peptide
PDPLKPTKRSFIEDL

SEQ ID NO: 818 - protein, 15 aa, Artificial sequence
  Synthetic peptide
DPLKPTKRSFIEDLL

SEQ ID NO: 819 - protein, 15 aa, Artificial sequence
  Synthetic peptide
PLKPTKRSFIEDLLF

SEQ ID NO: 820 - protein, 15 aa, Artificial sequence
  Synthetic peptide
LKPTKRSFIEDLLFN

SEQ ID NO: 821 - protein, 15 aa, Artificial sequence
  Synthetic peptide
KPTKRSFIEDLLFNK

SEQ ID NO: 822 - protein, 15 aa, Artificial sequence
  Synthetic peptide
PTKRSFIEDLLFNKV

SEQ ID NO: 823 - protein, 15 aa, Artificial sequence
  Synthetic peptide
TKRSFIEDLLFNKVT

SEQ ID NO: 824 - protein, 15 aa, Artificial sequence
  Synthetic peptide
KRSFIEDLLFNKVTL

SEQ ID NO: 825 - protein, 15 aa, Artificial sequence
  Synthetic peptide
RSFIEDLLFNKVTLA

SEQ ID NO: 826 - protein, 15 aa, Artificial sequence
  Synthetic peptide
SFIEDLLFNKVTLAD

SEQ ID NO: 827 - protein, 15 aa, Artificial sequence
  Synthetic peptide
FIEDLLFNKVTLADA

SEQ ID NO: 828 - protein, 15 aa, Artificial sequence
  Synthetic peptide
IEDLLFNKVTLADAG

SEQ ID NO: 829 - protein, 15 aa, Artificial sequence
  Synthetic peptide
EDLLFNKVTLADAGF

SEQ ID NO: 830 - protein, 15 aa, Artificial sequence
  Synthetic peptide
DLLFNKVTLADAGFM

SEQ ID NO: 831 - protein, 15 aa, Artificial sequence
  Synthetic peptide
LLFNKVTLADAGFMK

SEQ ID NO: 832 - protein, 15 aa, Artificial sequence
  Synthetic peptide
LFNKVTLADAGFMKQ

SEQ ID NO: 833 - protein, 15 aa, Artificial sequence
  Synthetic peptide
FNKVTLADAGFMKQY

SEQ ID NO: 834 - protein, 15 aa, Artificial sequence
  Synthetic peptide
NKVTLADAGFMKQYG

SEQ ID NO: 835 - protein, 15 aa, Artificial sequence
  Synthetic peptide
KVTLADAGFMKQYGE

SEQ ID NO: 836 - protein, 15 aa, Artificial sequence
  Synthetic peptide
VTLADAGFMKQYGEC

SEQ ID NO: 837 - protein, 15 aa, Artificial sequence
  Synthetic peptide
TLADAGFMKQYGECL

SEQ ID NO: 838 - protein, 15 aa, Artificial sequence
  Synthetic peptide
LADAGFMKQYGECLG

SEQ ID NO: 839 - protein, 15 aa, Artificial sequence
  Synthetic peptide
ADAGFMKQYGECLGD

SEQ ID NO: 840 - protein, 15 aa, Artificial sequence
  Synthetic peptide
DAGFMKQYGECLGDI

SEQ ID NO: 841 - protein, 15 aa, Artificial sequence
  Synthetic peptide
AGFMKQYGECLGDIN

SEQ ID NO: 842 - protein, 15 aa, Artificial sequence
  Synthetic peptide
GFMKQYGECLGDINA

SEQ ID NO: 843 - protein, 15 aa, Artificial sequence
  Synthetic peptide
FMKQYGECLGDINAR

SEQ ID NO: 844 - protein, 15 aa, Artificial sequence
  Synthetic peptide
MKQYGECLGDINARD

SEQ ID NO: 845 - protein, 15 aa, Artificial sequence
  Synthetic peptide
KQYGECLGDINARDL

SEQ ID NO: 846 - protein, 15 aa, Artificial sequence
  Synthetic peptide
QYGECLGDINARDLI

SEQ ID NO: 847 - protein, 15 aa, Artificial sequence
  Synthetic peptide
YGECLGDINARDLIC

SEQ ID NO: 848 - protein, 15 aa, Artificial sequence
  Synthetic peptide
GECLGDINARDLICA

SEQ ID NO: 849 - protein, 15 aa, Artificial sequence
  Synthetic peptide
ECLGDINARDLICAQ

SEQ ID NO: 850 - protein, 15 aa, Artificial sequence
  Synthetic peptide
CLGDINARDLICAQK

SEQ ID NO: 851 - protein, 15 aa, Artificial sequence
  Synthetic peptide
LGDINARDLICAQKF

SEQ ID NO: 852 - protein, 15 aa, Artificial sequence
  Synthetic peptide
GDINARDLICAQKFN

SEQ ID NO: 853 - protein, 15 aa, Artificial sequence
  Synthetic peptide
DINARDLICAQKFNG

SEQ ID NO: 854 - protein, 15 aa, Artificial sequence
  Synthetic peptide
INARDLICAQKFNGL

SEQ ID NO: 855 - protein, 15 aa, Artificial sequence
  Synthetic peptide
NARDLICAQKFNGLT

SEQ ID NO: 856 - protein, 15 aa, Artificial sequence
  Synthetic peptide
ARDLICAQKFNGLTV

SEQ ID NO: 857 - protein, 15 aa, Artificial sequence
  Synthetic peptide
RDLICAQKFNGLTVL

SEQ ID NO: 858 - protein, 15 aa, Artificial sequence
  Synthetic peptide
DLICAQKFNGLTVLP

SEQ ID NO: 859 - protein, 15 aa, Artificial sequence
  Synthetic peptide
LICAQKFNGLTVLPP

SEQ ID NO: 860 - protein, 15 aa, Artificial sequence
  Synthetic peptide
ICAQKFNGLTVLPPL

SEQ ID NO: 861 - protein, 15 aa, Artificial sequence
  Synthetic peptide
CAQKFNGLTVLPPLL

SEQ ID NO: 862 - protein, 15 aa, Artificial sequence
  Synthetic peptide
AQKFNGLTVLPPLLT

SEQ ID NO: 863 - protein, 15 aa, Artificial sequence
  Synthetic peptide
QKFNGLTVLPPLLTD

SEQ ID NO: 864 - protein, 15 aa, Artificial sequence
  Synthetic peptide
KFNGLTVLPPLLTDD

SEQ ID NO: 865 - protein, 15 aa, Artificial sequence
  Synthetic peptide
FNGLTVLPPLLTDDM

SEQ ID NO: 866 - protein, 15 aa, Artificial sequence
  Synthetic peptide
NGLTVLPPLLTDDMI

SEQ ID NO: 867 - protein, 15 aa, Artificial sequence
  Synthetic peptide
GLTVLPPLLTDDMIA

SEQ ID NO: 868 - protein, 15 aa, Artificial sequence
  Synthetic peptide
LTVLPPLLTDDMIAA

SEQ ID NO: 869 - protein, 15 aa, Artificial sequence
  Synthetic peptide
TVLPPLLTDDMIAAY

SEQ ID NO: 870 - protein, 15 aa, Artificial sequence
  Synthetic peptide
VLPPLLTDDMIAAYT

SEQ ID NO: 871 - protein, 15 aa, Artificial sequence
  Synthetic peptide
LPPLLTDDMIAAYTA

SEQ ID NO: 872 - protein, 15 aa, Artificial sequence
  Synthetic peptide
PPLLTDDMIAAYTAA

SEQ ID NO: 873 - protein, 15 aa, Artificial sequence
  Synthetic peptide
PLLTDDMIAAYTAAL

SEQ ID NO: 874 - protein, 15 aa, Artificial sequence
  Synthetic peptide
LLTDDMIAAYTAALV

SEQ ID NO: 875 - protein, 15 aa, Artificial sequence
  Synthetic peptide
LTDDMIAAYTAALVS

SEQ ID NO: 876 - protein, 15 aa, Artificial sequence
  Synthetic peptide
TDDMIAAYTAALVSG

SEQ ID NO: 877 - protein, 15 aa, Artificial sequence
  Synthetic peptide
DDMIAAYTAALVSGT

SEQ ID NO: 878 - protein, 15 aa, Artificial sequence
  Synthetic peptide
DMIAAYTAALVSGTA

SEQ ID NO: 879 - protein, 15 aa, Artificial sequence
  Synthetic peptide
MIAAYTAALVSGTAT

SEQ ID NO: 880 - protein, 15 aa, Artificial sequence
  Synthetic peptide
IAAYTAALVSGTATA

SEQ ID NO: 881 - protein, 15 aa, Artificial sequence
  Synthetic peptide
AAYTAALVSGTATAG

SEQ ID NO: 882 - protein, 15 aa, Artificial sequence
  Synthetic peptide
AYTAALVSGTATAGW

SEQ ID NO: 883 - protein, 15 aa, Artificial sequence
  Synthetic peptide
YTAALVSGTATAGWT

SEQ ID NO: 884 - protein, 15 aa, Artificial sequence
  Synthetic peptide
TAALVSGTATAGWTF

SEQ ID NO: 885 - protein, 15 aa, Artificial sequence
  Synthetic peptide
AALVSGTATAGWTFG

SEQ ID NO: 886 - protein, 15 aa, Artificial sequence
  Synthetic peptide
ALVSGTATAGWTFGA

SEQ ID NO: 887 - protein, 15 aa, Artificial sequence
  Synthetic peptide
LVSGTATAGWTFGAG

SEQ ID NO: 888 - protein, 15 aa, Artificial sequence
  Synthetic peptide
VSGTATAGWTFGAGA

SEQ ID NO: 889 - protein, 15 aa, Artificial sequence
  Synthetic peptide
SGTATAGWTFGAGAA

SEQ ID NO: 890 - protein, 15 aa, Artificial sequence
  Synthetic peptide
GTATAGWTFGAGAAL

SEQ ID NO: 891 - protein, 15 aa, Artificial sequence
  Synthetic peptide
TATAGWTFGAGAALQ

SEQ ID NO: 892 - protein, 15 aa, Artificial sequence
  Synthetic peptide
ATAGWTFGAGAALQI

SEQ ID NO: 893 - protein, 15 aa, Artificial sequence
  Synthetic peptide
TAGWTFGAGAALQIP

SEQ ID NO: 894 - protein, 15 aa, Artificial sequence
  Synthetic peptide
AGWTFGAGAALQIPF

SEQ ID NO: 895 - protein, 15 aa, Artificial sequence
  Synthetic peptide
GWTFGAGAALQIPFA

SEQ ID NO: 896 - protein, 15 aa, Artificial sequence
  Synthetic peptide
WTFGAGAALQIPFAM

SEQ ID NO: 897 - protein, 15 aa, Artificial sequence
  Synthetic peptide
TFGAGAALQIPFAMQ

SEQ ID NO: 898 - protein, 15 aa, Artificial sequence
  Synthetic peptide
FGAGAALQIPFAMQM

SEQ ID NO: 899 - protein, 15 aa, Artificial sequence
  Synthetic peptide
GAGAALQIPFAMQMA

SEQ ID NO: 900 - protein, 15 aa, Artificial sequence
  Synthetic peptide
AGAALQIPFAMQMAY

SEQ ID NO: 901 - protein, 15 aa, Artificial sequence
  Synthetic peptide
GAALQIPFAMQMAYR

SEQ ID NO: 902 - protein, 15 aa, Artificial sequence
  Synthetic peptide
AALQIPFAMQMAYRF

SEQ ID NO: 903 - protein, 15 aa, Artificial sequence
  Synthetic peptide
ALQIPFAMQMAYRFN

SEQ ID NO: 904 - protein, 15 aa, Artificial sequence
  Synthetic peptide
LQIPFAMQMAYRFNG

SEQ ID NO: 905 - protein, 15 aa, Artificial sequence
  Synthetic peptide
QIPFAMQMAYRFNGI

SEQ ID NO: 906 - protein, 15 aa, Artificial sequence
  Synthetic peptide
IPFAMQMAYRFNGIG

SEQ ID NO: 907 - protein, 15 aa, Artificial sequence
  Synthetic peptide
PFAMQMAYRFNGIGV

SEQ ID NO: 908 - protein, 15 aa, Artificial sequence
  Synthetic peptide
FAMQMAYRFNGIGVT

SEQ ID NO: 909 - protein, 15 aa, Artificial sequence
  Synthetic peptide
AMQMAYRFNGIGVTQ

SEQ ID NO: 910 - protein, 15 aa, Artificial sequence
  Synthetic peptide
MQMAYRFNGIGVTQN

SEQ ID NO: 911 - protein, 15 aa, Artificial sequence
  Synthetic peptide
QMAYRFNGIGVTQNV

SEQ ID NO: 912 - protein, 15 aa, Artificial sequence
  Synthetic peptide
MAYRFNGIGVTQNVL

SEQ ID NO: 913 - protein, 15 aa, Artificial sequence
  Synthetic peptide
AYRFNGIGVTQNVLY

SEQ ID NO: 914 - protein, 15 aa, Artificial sequence
  Synthetic peptide
YRFNGIGVTQNVLYE

SEQ ID NO: 915 - protein, 15 aa, Artificial sequence
  Synthetic peptide
RFNGIGVTQNVLYEN

SEQ ID NO: 916 - protein, 15 aa, Artificial sequence
  Synthetic peptide
FNGIGVTQNVLYENQ

SEQ ID NO: 917 - protein, 15 aa, Artificial sequence
  Synthetic peptide
NGIGVTQNVLYENQK

SEQ ID NO: 918 - protein, 15 aa, Artificial sequence
  Synthetic peptide
GIGVTQNVLYENQKQ

SEQ ID NO: 919 - protein, 15 aa, Artificial sequence
  Synthetic peptide
IGVTQNVLYENQKQI

SEQ ID NO: 920 - protein, 15 aa, Artificial sequence
  Synthetic peptide
GVTQNVLYENQKQIA

SEQ ID NO: 921 - protein, 15 aa, Artificial sequence
  Synthetic peptide
VTQNVLYENQKQIAN

SEQ ID NO: 922 - protein, 15 aa, Artificial sequence
  Synthetic peptide
TQNVLYENQKQIANQ

SEQ ID NO: 923 - protein, 15 aa, Artificial sequence
  Synthetic peptide
QNVLYENQKQIANQF

SEQ ID NO: 924 - protein, 15 aa, Artificial sequence
  Synthetic peptide
NVLYENQKQIANQFN

SEQ ID NO: 925 - protein, 15 aa, Artificial sequence
  Synthetic peptide
VLYENQKQIANQFNK

SEQ ID NO: 926 - protein, 15 aa, Artificial sequence
  Synthetic peptide
LYENQKQIANQFNKA

SEQ ID NO: 927 - protein, 15 aa, Artificial sequence
  Synthetic peptide
YENQKQIANQFNKAI

SEQ ID NO: 928 - protein, 15 aa, Artificial sequence
  Synthetic peptide
ENQKQIANQFNKAIS

SEQ ID NO: 929 - protein, 15 aa, Artificial sequence
  Synthetic peptide
NQKQIANQFNKAISQ

SEQ ID NO: 930 - protein, 15 aa, Artificial sequence
  Synthetic peptide
QKQIANQFNKAISQI

SEQ ID NO: 931 - protein, 15 aa, Artificial sequence
  Synthetic peptide
KQIANQFNKAISQIQ

SEQ ID NO: 932 - protein, 15 aa, Artificial sequence
  Synthetic peptide
QIANQFNKAISQIQE

SEQ ID NO: 933 - protein, 15 aa, Artificial sequence
  Synthetic peptide
IANQFNKAISQIQES

SEQ ID NO: 934 - protein, 15 aa, Artificial sequence
  Synthetic peptide
ANQFNKAISQIQESL

SEQ ID NO: 935 - protein, 15 aa, Artificial sequence
  Synthetic peptide
NQFNKAISQIQESLT

SEQ ID NO: 936 - protein, 15 aa, Artificial sequence
  Synthetic peptide
QFNKAISQIQESLTT

SEQ ID NO: 937 - protein, 15 aa, Artificial sequence
  Synthetic peptide
FNKAISQIQESLTTT

SEQ ID NO: 938 - protein, 15 aa, Artificial sequence
  Synthetic peptide
NKAISQIQESLTTTS

SEQ ID NO: 939 - protein, 15 aa, Artificial sequence
  Synthetic peptide
KAISQIQESLTTTST

SEQ ID NO: 940 - protein, 15 aa, Artificial sequence
  Synthetic peptide
AISQIQESLTTTSTA

SEQ ID NO: 941 - protein, 15 aa, Artificial sequence
  Synthetic peptide
ISQIQESLTTTSTAL

SEQ ID NO: 942 - protein, 15 aa, Artificial sequence
  Synthetic peptide
SQIQESLTTTSTALG

SEQ ID NO: 943 - protein, 15 aa, Artificial sequence
  Synthetic peptide
QIQESLTTTSTALGK

SEQ ID NO: 944 - protein, 15 aa, Artificial sequence
  Synthetic peptide
IQESLTTTSTALGKL

SEQ ID NO: 945 - protein, 15 aa, Artificial sequence
  Synthetic peptide
QESLTTTSTALGKLQ

SEQ ID NO: 946 - protein, 15 aa, Artificial sequence
  Synthetic peptide
ESLTTTSTALGKLQD

SEQ ID NO: 947 - protein, 15 aa, Artificial sequence
  Synthetic peptide
SLTTTSTALGKLQDV

SEQ ID NO: 948 - protein, 15 aa, Artificial sequence
  Synthetic peptide
LTTTSTALGKLQDVV

SEQ ID NO: 949 - protein, 15 aa, Artificial sequence
  Synthetic peptide
TTTSTALGKLQDVVN

SEQ ID NO: 950 - protein, 15 aa, Artificial sequence
  Synthetic peptide
TTSTALGKLQDVVNQ

SEQ ID NO: 951 - protein, 15 aa, Artificial sequence
  Synthetic peptide
TSTALGKLQDVVNQN

SEQ ID NO: 952 - protein, 15 aa, Artificial sequence
  Synthetic peptide
STALGKLQDVVNQNA

SEQ ID NO: 953 - protein, 15 aa, Artificial sequence
  Synthetic peptide
TALGKLQDVVNQNAQ

SEQ ID NO: 954 - protein, 15 aa, Artificial sequence
  Synthetic peptide
ALGKLQDVVNQNAQA

SEQ ID NO: 955 - protein, 15 aa, Artificial sequence
  Synthetic peptide
LGKLQDVVNQNAQAL

SEQ ID NO: 956 - protein, 15 aa, Artificial sequence
  Synthetic peptide
GKLQDVVNQNAQALN

SEQ ID NO: 957 - protein, 15 aa, Artificial sequence
  Synthetic peptide
KLQDVVNQNAQALNT

SEQ ID NO: 958 - protein, 15 aa, Artificial sequence
  Synthetic peptide
LQDVVNQNAQALNTL

SEQ ID NO: 959 - protein, 15 aa, Artificial sequence
  Synthetic peptide
QDVVNQNAQALNTLV

SEQ ID NO: 960 - protein, 15 aa, Artificial sequence
  Synthetic peptide
DVVNQNAQALNTLVK

SEQ ID NO: 961 - protein, 15 aa, Artificial sequence
  Synthetic peptide
VVNQNAQALNTLVKQ

SEQ ID NO: 962 - protein, 15 aa, Artificial sequence
  Synthetic peptide
VNQNAQALNTLVKQL

SEQ ID NO: 963 - protein, 15 aa, Artificial sequence
  Synthetic peptide
NQNAQALNTLVKQLS

SEQ ID NO: 964 - protein, 15 aa, Artificial sequence
  Synthetic peptide
QNAQALNTLVKQLSS

SEQ ID NO: 965 - protein, 15 aa, Artificial sequence
  Synthetic peptide
NAQALNTLVKQLSSN

SEQ ID NO: 966 - protein, 15 aa, Artificial sequence
  Synthetic peptide
AQALNTLVKQLSSNF

SEQ ID NO: 967 - protein, 15 aa, Artificial sequence
  Synthetic peptide
QALNTLVKQLSSNFG

SEQ ID NO: 968 - protein, 15 aa, Artificial sequence
  Synthetic peptide
ALNTLVKQLSSNFGA

SEQ ID NO: 969 - protein, 15 aa, Artificial sequence
  Synthetic peptide
LNTLVKQLSSNFGAI

SEQ ID NO: 970 - protein, 15 aa, Artificial sequence
  Synthetic peptide
NTLVKQLSSNFGAIS

SEQ ID NO: 971 - protein, 15 aa, Artificial sequence
  Synthetic peptide
TLVKQLSSNFGAISS

SEQ ID NO: 972 - protein, 15 aa, Artificial sequence
  Synthetic peptide
LVKQLSSNFGAISSV

SEQ ID NO: 973 - protein, 15 aa, Artificial sequence
  Synthetic peptide
VKQLSSNFGAISSVL

SEQ ID NO: 974 - protein, 15 aa, Artificial sequence
  Synthetic peptide
KQLSSNFGAISSVLN

SEQ ID NO: 975 - protein, 15 aa, Artificial sequence
  Synthetic peptide
QLSSNFGAISSVLND

SEQ ID NO: 976 - protein, 15 aa, Artificial sequence
  Synthetic peptide
LSSNFGAISSVLNDI

SEQ ID NO: 977 - protein, 15 aa, Artificial sequence
  Synthetic peptide
SSNFGAISSVLNDIL

SEQ ID NO: 978 - protein, 15 aa, Artificial sequence
  Synthetic peptide
SNFGAISSVLNDILS

SEQ ID NO: 979 - protein, 15 aa, Artificial sequence
  Synthetic peptide
NFGAISSVLNDILSR

SEQ ID NO: 980 - protein, 15 aa, Artificial sequence
  Synthetic peptide
FGAISSVLNDILSRL

SEQ ID NO: 981 - protein, 15 aa, Artificial sequence
  Synthetic peptide
GAISSVLNDILSRLD

SEQ ID NO: 982 - protein, 15 aa, Artificial sequence
  Synthetic peptide
AISSVLNDILSRLDK

SEQ ID NO: 983 - protein, 15 aa, Artificial sequence
  Synthetic peptide
ISSVLNDILSRLDKV

SEQ ID NO: 984 - protein, 15 aa, Artificial sequence
  Synthetic peptide
SSVLNDILSRLDKVE

SEQ ID NO: 985 - protein, 15 aa, Artificial sequence
  Synthetic peptide
SVLNDILSRLDKVEA

SEQ ID NO: 986 - protein, 15 aa, Artificial sequence
  Synthetic peptide
VLNDILSRLDKVEAE

SEQ ID NO: 987 - protein, 15 aa, Artificial sequence
  Synthetic peptide
LNDILSRLDKVEAEV

SEQ ID NO: 988 - protein, 15 aa, Artificial sequence
  Synthetic peptide
NDILSRLDKVEAEVQ

SEQ ID NO: 989 - protein, 15 aa, Artificial sequence
  Synthetic peptide
DILSRLDKVEAEVQI

SEQ ID NO: 990 - protein, 15 aa, Artificial sequence
  Synthetic peptide
ILSRLDKVEAEVQID

SEQ ID NO: 991 - protein, 15 aa, Artificial sequence
  Synthetic peptide
LSRLDKVEAEVQIDR

SEQ ID NO: 992 - protein, 15 aa, Artificial sequence
  Synthetic peptide
SRLDKVEAEVQIDRL

SEQ ID NO: 993 - protein, 15 aa, Artificial sequence
  Synthetic peptide
RLDKVEAEVQIDRLI

SEQ ID NO: 994 - protein, 15 aa, Artificial sequence
  Synthetic peptide
LDKVEAEVQIDRLIT

SEQ ID NO: 995 - protein, 15 aa, Artificial sequence
  Synthetic peptide
DKVEAEVQIDRLITG

SEQ ID NO: 996 - protein, 15 aa, Artificial sequence
  Synthetic peptide
KVEAEVQIDRLITGR

SEQ ID NO: 997 - protein, 15 aa, Artificial sequence
  Synthetic peptide
VEAEVQIDRLITGRL

SEQ ID NO: 998 - protein, 15 aa, Artificial sequence
  Synthetic peptide
EAEVQIDRLITGRLQ

SEQ ID NO: 999 - protein, 15 aa, Artificial sequence
  Synthetic peptide
AEVQIDRLITGRLQS

SEQ ID NO: 1000 - protein, 15 aa, Artificial sequence
  Synthetic peptide
EVQIDRLITGRLQSL

SEQ ID NO: 1001 - protein, 15 aa, Artificial sequence
  Synthetic peptide
VQIDRLITGRLQSLQ

SEQ ID NO: 1002 - protein, 15 aa, Artificial sequence
  Synthetic peptide
QIDRLITGRLQSLQT

SEQ ID NO: 1003 - protein, 15 aa, Artificial sequence
  Synthetic peptide
IDRLITGRLQSLQTY

SEQ ID NO: 1004 - protein, 15 aa, Artificial sequence
  Synthetic peptide
DRLITGRLQSLQTYV

SEQ ID NO: 1005 - protein, 15 aa, Artificial sequence
  Synthetic peptide
RLITGRLQSLQTYVT

SEQ ID NO: 1006 - protein, 15 aa, Artificial sequence
  Synthetic peptide
LITGRLQSLQTYVTQ

SEQ ID NO: 1007 - protein, 15 aa, Artificial sequence
  Synthetic peptide
ITGRLQSLQTYVTQQ

SEQ ID NO: 1008 - protein, 15 aa, Artificial sequence
  Synthetic peptide
TGRLQSLQTYVTQQL

SEQ ID NO: 1009 - protein, 15 aa, Artificial sequence
  Synthetic peptide
GRLQSLQTYVTQQLI

SEQ ID NO: 1010 - protein, 15 aa, Artificial sequence
  Synthetic peptide
RLQSLQTYVTQQLIR

SEQ ID NO: 1011 - protein, 15 aa, Artificial sequence
  Synthetic peptide
LQSLQTYVTQQLIRA

SEQ ID NO: 1012 - protein, 15 aa, Artificial sequence
  Synthetic peptide
QSLQTYVTQQLIRAA

SEQ ID NO: 1013 - protein, 15 aa, Artificial sequence
  Synthetic peptide
SLQTYVTQQLIRAAE

SEQ ID NO: 1014 - protein, 15 aa, Artificial sequence
  Synthetic peptide
LQTYVTQQLIRAAEI

SEQ ID NO: 1015 - protein, 15 aa, Artificial sequence
  Synthetic peptide
QTYVTQQLIRAAEIR

SEQ ID NO: 1016 - protein, 15 aa, Artificial sequence
  Synthetic peptide
TYVTQQLIRAAEIRA

SEQ ID NO: 1017 - protein, 15 aa, Artificial sequence
  Synthetic peptide
YVTQQLIRAAEIRAS

SEQ ID NO: 1018 - protein, 15 aa, Artificial sequence
  Synthetic peptide
VTQQLIRAAEIRASA

SEQ ID NO: 1019 - protein, 15 aa, Artificial sequence
  Synthetic peptide
TQQLIRAAEIRASAN

SEQ ID NO: 1020 - protein, 15 aa, Artificial sequence
  Synthetic peptide
QQLIRAAEIRASANL

SEQ ID NO: 1021 - protein, 15 aa, Artificial sequence
  Synthetic peptide
QLIRAAEIRASANLA

SEQ ID NO: 1022 - protein, 15 aa, Artificial sequence
  Synthetic peptide
LIRAAEIRASANLAA

SEQ ID NO: 1023 - protein, 15 aa, Artificial sequence
  Synthetic peptide
IRAAEIRASANLAAT

SEQ ID NO: 1024 - protein, 15 aa, Artificial sequence
  Synthetic peptide
EIRASANLAATKMSE

SEQ ID NO: 1025 - protein, 15 aa, Artificial sequence
  Synthetic peptide
IRASANLAATKMSEC

SEQ ID NO: 1026 - protein, 15 aa, Artificial sequence
  Synthetic peptide
RASANLAATKMSECV

SEQ ID NO: 1027 - protein, 15 aa, Artificial sequence
  Synthetic peptide
ASANLAATKMSECVL

SEQ ID NO: 1028 - protein, 15 aa, Artificial sequence
  Synthetic peptide
SANLAATKMSECVLG

SEQ ID NO: 1029 - protein, 15 aa, Artificial sequence
  Synthetic peptide
ANLAATKMSECVLGQ

SEQ ID NO: 1030 - protein, 15 aa, Artificial sequence
  Synthetic peptide
NLAATKMSECVLGQS

SEQ ID NO: 1031 - protein, 15 aa, Artificial sequence
  Synthetic peptide
LAATKMSECVLGQSK

SEQ ID NO: 1032 - protein, 15 aa, Artificial sequence
  Synthetic peptide
AATKMSECVLGQSKR

SEQ ID NO: 1033 - protein, 15 aa, Artificial sequence
  Synthetic peptide
ATKMSECVLGQSKRV

SEQ ID NO: 1034 - protein, 15 aa, Artificial sequence
  Synthetic peptide
TKMSECVLGQSKRVD

SEQ ID NO: 1035 - protein, 15 aa, Artificial sequence
  Synthetic peptide
KMSECVLGQSKRVDF

SEQ ID NO: 1036 - protein, 15 aa, Artificial sequence
  Synthetic peptide
MSECVLGQSKRVDFC

SEQ ID NO: 1037 - protein, 15 aa, Artificial sequence
  Synthetic peptide
SECVLGQSKRVDFCG

SEQ ID NO: 1038 - protein, 15 aa, Artificial sequence
  Synthetic peptide
ECVLGQSKRVDFCGK

SEQ ID NO: 1039 - protein, 15 aa, Artificial sequence
  Synthetic peptide
CVLGQSKRVDFCGKG

SEQ ID NO: 1040 - protein, 15 aa, Artificial sequence
  Synthetic peptide
VLGQSKRVDFCGKGY

SEQ ID NO: 1041 - protein, 15 aa, Artificial sequence
  Synthetic peptide
LGQSKRVDFCGKGYH

SEQ ID NO: 1042 - protein, 15 aa, Artificial sequence
  Synthetic peptide
QSKRVDFCGKGYHLM

SEQ ID NO: 1043 - protein, 15 aa, Artificial sequence
  Synthetic peptide
SKRVDFCGKGYHLMS

SEQ ID NO: 1044 - protein, 15 aa, Artificial sequence
  Synthetic peptide
KRVDFCGKGYHLMSF

SEQ ID NO: 1045 - protein, 15 aa, Artificial sequence
  Synthetic peptide
RVDFCGKGYHLMSFP

SEQ ID NO: 1046 - protein, 15 aa, Artificial sequence
  Synthetic peptide
VDFCGKGYHLMSFPQ

SEQ ID NO: 1047 - protein, 15 aa, Artificial sequence
  Synthetic peptide
DFCGKGYHLMSFPQA

SEQ ID NO: 1048 - protein, 15 aa, Artificial sequence
  Synthetic peptide
FCGKGYHLMSFPQAA

SEQ ID NO: 1049 - protein, 15 aa, Artificial sequence
  Synthetic peptide
CGKGYHLMSFPQAAP

SEQ ID NO: 1050 - protein, 15 aa, Artificial sequence
  Synthetic peptide
GKGYHLMSFPQAAPH

SEQ ID NO: 1051 - protein, 15 aa, Artificial sequence
  Synthetic peptide
KGYHLMSFPQAAPHG

SEQ ID NO: 1052 - protein, 15 aa, Artificial sequence
  Synthetic peptide
GYHLMSFPQAAPHGV

SEQ ID NO: 1053 - protein, 15 aa, Artificial sequence
  Synthetic peptide
LMSFPQAAPHGVVFL

SEQ ID NO: 1054 - protein, 15 aa, Artificial sequence
  Synthetic peptide
MSFPQAAPHGVVFLH

SEQ ID NO: 1055 - protein, 15 aa, Artificial sequence
  Synthetic peptide
FPQAAPHGVVFLHVT

SEQ ID NO: 1056 - protein, 15 aa, Artificial sequence
  Synthetic peptide
PQAAPHGVVFLHVTY

SEQ ID NO: 1057 - protein, 15 aa, Artificial sequence
  Synthetic peptide
QAAPHGVVFLHVTYV

SEQ ID NO: 1058 - protein, 15 aa, Artificial sequence
  Synthetic peptide
AAPHGVVFLHVTYVP

SEQ ID NO: 1059 - protein, 15 aa, Artificial sequence
  Synthetic peptide
APHGVVFLHVTYVPS

SEQ ID NO: 1060 - protein, 15 aa, Artificial sequence
  Synthetic peptide
PHGVVFLHVTYVPSQ

SEQ ID NO: 1061 - protein, 15 aa, Artificial sequence
  Synthetic peptide
HGVVFLHVTYVPSQE

SEQ ID NO: 1062 - protein, 15 aa, Artificial sequence
  Synthetic peptide
GVVFLHVTYVPSQER

SEQ ID NO: 1063 - protein, 15 aa, Artificial sequence
  Synthetic peptide
VVFLHVTYVPSQERN

SEQ ID NO: 1064 - protein, 15 aa, Artificial sequence
  Synthetic peptide
VFLHVTYVPSQERNF

SEQ ID NO: 1065 - protein, 15 aa, Artificial sequence
  Synthetic peptide
FLHVTYVPSQERNFT

SEQ ID NO: 1066 - protein, 15 aa, Artificial sequence
  Synthetic peptide
LHVTYVPSQERNFTT

SEQ ID NO: 1067 - protein, 15 aa, Artificial sequence
  Synthetic peptide
HVTYVPSQERNFTTA

SEQ ID NO: 1068 - protein, 15 aa, Artificial sequence
  Synthetic peptide
VTYVPSQERNFTTAP

SEQ ID NO: 1069 - protein, 15 aa, Artificial sequence
  Synthetic peptide
PSQERNFTTAPAICH

SEQ ID NO: 1070 - protein, 15 aa, Artificial sequence
  Synthetic peptide
QERNFTTAPAICHEG

SEQ ID NO: 1071 - protein, 15 aa, Artificial sequence
  Synthetic peptide
ERNFTTAPAICHEGK

SEQ ID NO: 1072 - protein, 15 aa, Artificial sequence
  Synthetic peptide
RNFTTAPAICHEGKA

SEQ ID NO: 1073 - protein, 15 aa, Artificial sequence
  Synthetic peptide
FTTAPAICHEGKAYF

SEQ ID NO: 1074 - protein, 15 aa, Artificial sequence
  Synthetic peptide
TTAPAICHEGKAYFP

SEQ ID NO: 1075 - protein, 15 aa, Artificial sequence
  Synthetic peptide
TAPAICHEGKAYFPR

SEQ ID NO: 1076 - protein, 15 aa, Artificial sequence
  Synthetic peptide
APAICHEGKAYFPRE

SEQ ID NO: 1077 - protein, 15 aa, Artificial sequence
  Synthetic peptide
PAICHEGKAYFPREG

SEQ ID NO: 1078 - protein, 15 aa, Artificial sequence
  Synthetic peptide
AICHEGKAYFPREGV

SEQ ID NO: 1079 - protein, 15 aa, Artificial sequence
  Synthetic peptide
ICHEGKAYFPREGVF

SEQ ID NO: 1080 - protein, 15 aa, Artificial sequence
  Synthetic peptide
CHEGKAYFPREGVFV

SEQ ID NO: 1081 - protein, 15 aa, Artificial sequence
  Synthetic peptide
HEGKAYFPREGVFVF

SEQ ID NO: 1082 - protein, 15 aa, Artificial sequence
  Synthetic peptide
EGKAYFPREGVFVFN

SEQ ID NO: 1083 - protein, 15 aa, Artificial sequence
  Synthetic peptide
GKAYFPREGVFVFNG

SEQ ID NO: 1084 - protein, 15 aa, Artificial sequence
  Synthetic peptide
KAYFPREGVFVFNGT

SEQ ID NO: 1085 - protein, 15 aa, Artificial sequence
  Synthetic peptide
AYFPREGVFVFNGTS

SEQ ID NO: 1086 - protein, 15 aa, Artificial sequence
  Synthetic peptide
YFPREGVFVFNGTSW

SEQ ID NO: 1087 - protein, 15 aa, Artificial sequence
  Synthetic peptide
FPREGVFVFNGTSWF

SEQ ID NO: 1088 - protein, 15 aa, Artificial sequence
  Synthetic peptide
PREGVFVFNGTSWFI

SEQ ID NO: 1089 - protein, 15 aa, Artificial sequence
  Synthetic peptide
REGVFVFNGTSWFIT

SEQ ID NO: 1090 - protein, 15 aa, Artificial sequence
  Synthetic peptide
EGVFVFNGTSWFITQ

SEQ ID NO: 1091 - protein, 15 aa, Artificial sequence
  Synthetic peptide
GVFVFNGTSWFITQR

SEQ ID NO: 1092 - protein, 15 aa, Artificial sequence
  Synthetic peptide
VFVFNGTSWFITQRN

SEQ ID NO: 1093 - protein, 15 aa, Artificial sequence
  Synthetic peptide
FVFNGTSWFITQRNF

SEQ ID NO: 1094 - protein, 15 aa, Artificial sequence
  Synthetic peptide
VFNGTSWFITQRNFF

SEQ ID NO: 1095 - protein, 15 aa, Artificial sequence
  Synthetic peptide
FNGTSWFITQRNFFS

SEQ ID NO: 1096 - protein, 15 aa, Artificial sequence
  Synthetic peptide
NGTSWFITQRNFFSP

SEQ ID NO: 1097 - protein, 15 aa, Artificial sequence
  Synthetic peptide
GTSWFITQRNFFSPQ

SEQ ID NO: 1098 - protein, 15 aa, Artificial sequence
  Synthetic peptide
TSWFITQRNFFSPQI

SEQ ID NO: 1099 - protein, 15 aa, Artificial sequence
  Synthetic peptide
SWFITQRNFFSPQII

SEQ ID NO: 1100 - protein, 15 aa, Artificial sequence
  Synthetic peptide
WFITQRNFFSPQIIT

SEQ ID NO: 1101 - protein, 15 aa, Artificial sequence
  Synthetic peptide
FITQRNFFSPQIITT

SEQ ID NO: 1102 - protein, 15 aa, Artificial sequence
  Synthetic peptide
ITQRNFFSPQIITTD

SEQ ID NO: 1103 - protein, 15 aa, Artificial sequence
  Synthetic peptide
TQRNFFSPQIITTDN

SEQ ID NO: 1104 - protein, 15 aa, Artificial sequence
  Synthetic peptide
QRNFFSPQIITTDNT

SEQ ID NO: 1105 - protein, 15 aa, Artificial sequence
  Synthetic peptide
RNFFSPQIITTDNTF

SEQ ID NO: 1106 - protein, 15 aa, Artificial sequence
  Synthetic peptide
NFFSPQIITTDNTFV

SEQ ID NO: 1107 - protein, 15 aa, Artificial sequence
  Synthetic peptide
FFSPQIITTDNTFVS

SEQ ID NO: 1108 - protein, 15 aa, Artificial sequence
  Synthetic peptide
FSPQIITTDNTFVSG

SEQ ID NO: 1109 - protein, 15 aa, Artificial sequence
  Synthetic peptide
SPQIITTDNTFVSGN

SEQ ID NO: 1110 - protein, 15 aa, Artificial sequence
  Synthetic peptide
PQIITTDNTFVSGNC

SEQ ID NO: 1111 - protein, 15 aa, Artificial sequence
  Synthetic peptide
QIITTDNTFVSGNCD

SEQ ID NO: 1112 - protein, 15 aa, Artificial sequence
  Synthetic peptide
IITTDNTFVSGNCDV

SEQ ID NO: 1113 - protein, 15 aa, Artificial sequence
  Synthetic peptide
ITTDNTFVSGNCDVV

SEQ ID NO: 1114 - protein, 15 aa, Artificial sequence
  Synthetic peptide
TTDNTFVSGNCDVVI

SEQ ID NO: 1115 - protein, 15 aa, Artificial sequence
  Synthetic peptide
TDNTFVSGNCDVVIG

SEQ ID NO: 1116 - protein, 15 aa, Artificial sequence
  Synthetic peptide
DNTFVSGNCDVVIGI

SEQ ID NO: 1117 - protein, 15 aa, Artificial sequence
  Synthetic peptide
NTFVSGNCDVVIGII

SEQ ID NO: 1118 - protein, 15 aa, Artificial sequence
  Synthetic peptide
TFVSGNCDVVIGIIN

SEQ ID NO: 1119 - protein, 15 aa, Artificial sequence
  Synthetic peptide
FVSGNCDVVIGIINN

SEQ ID NO: 1120 - protein, 15 aa, Artificial sequence
  Synthetic peptide
VSGNCDVVIGIINNT

SEQ ID NO: 1121 - protein, 15 aa, Artificial sequence
  Synthetic peptide
SGNCDVVIGIINNTV

SEQ ID NO: 1122 - protein, 15 aa, Artificial sequence
  Synthetic peptide
GNCDVVIGIINNTVY

SEQ ID NO: 1123 - protein, 15 aa, Artificial sequence
  Synthetic peptide
NCDVVIGIINNTVYD

SEQ ID NO: 1124 - protein, 15 aa, Artificial sequence
  Synthetic peptide
CDVVIGIINNTVYDP

SEQ ID NO: 1125 - protein, 15 aa, Artificial sequence
  Synthetic peptide
DVVIGIINNTVYDPL

SEQ ID NO: 1126 - protein, 15 aa, Artificial sequence
  Synthetic peptide
VVIGIINNTVYDPLQ

SEQ ID NO: 1127 - protein, 15 aa, Artificial sequence
  Synthetic peptide
VIGIINNTVYDPLQP

SEQ ID NO: 1128 - protein, 15 aa, Artificial sequence
  Synthetic peptide
IGIINNTVYDPLQPE

SEQ ID NO: 1129 - protein, 15 aa, Artificial sequence
  Synthetic peptide
GIINNTVYDPLQPEL

SEQ ID NO: 1130 - protein, 15 aa, Artificial sequence
  Synthetic peptide
IINNTVYDPLQPELD

SEQ ID NO: 1131 - protein, 15 aa, Artificial sequence
  Synthetic peptide
INNTVYDPLQPELDS

SEQ ID NO: 1132 - protein, 15 aa, Artificial sequence
  Synthetic peptide
NNTVYDPLQPELDSF

SEQ ID NO: 1133 - protein, 15 aa, Artificial sequence
  Synthetic peptide
NTVYDPLQPELDSFK

SEQ ID NO: 1134 - protein, 15 aa, Artificial sequence
  Synthetic peptide
TVYDPLQPELDSFKE

SEQ ID NO: 1135 - protein, 15 aa, Artificial sequence
  Synthetic peptide
VYDPLQPELDSFKEE

SEQ ID NO: 1136 - protein, 15 aa, Artificial sequence
  Synthetic peptide
YDPLQPELDSFKEEL

SEQ ID NO: 1137 - protein, 15 aa, Artificial sequence
  Synthetic peptide
DPLQPELDSFKEELD

SEQ ID NO: 1138 - protein, 15 aa, Artificial sequence
  Synthetic peptide
PLQPELDSFKEELDK

SEQ ID NO: 1139 - protein, 15 aa, Artificial sequence
  Synthetic peptide
LQPELDSFKEELDKY

SEQ ID NO: 1140 - protein, 15 aa, Artificial sequence
  Synthetic peptide
QPELDSFKEELDKYF

SEQ ID NO: 1141 - protein, 15 aa, Artificial sequence
  Synthetic peptide
PELDSFKEELDKYFK

SEQ ID NO: 1142 - protein, 15 aa, Artificial sequence
  Synthetic peptide
ELDSFKEELDKYFKN

SEQ ID NO: 1143 - protein, 15 aa, Artificial sequence
  Synthetic peptide
LDSFKEELDKYFKNH

SEQ ID NO: 1144 - protein, 15 aa, Artificial sequence
  Synthetic peptide
DSFKEELDKYFKNHT

SEQ ID NO: 1145 - protein, 15 aa, Artificial sequence
  Synthetic peptide
SFKEELDKYFKNHTS

SEQ ID NO: 1146 - protein, 15 aa, Artificial sequence
  Synthetic peptide
FKEELDKYFKNHTSP

SEQ ID NO: 1147 - protein, 15 aa, Artificial sequence
  Synthetic peptide
ELDKYFKNHTSPDVD

SEQ ID NO: 1148 - protein, 15 aa, Artificial sequence
  Synthetic peptide
LDKYFKNHTSPDVDL

SEQ ID NO: 1149 - protein, 15 aa, Artificial sequence
  Synthetic peptide
DKYFKNHTSPDVDLG

SEQ ID NO: 1150 - protein, 15 aa, Artificial sequence
  Synthetic peptide
KYFKNHTSPDVDLGD

SEQ ID NO: 1151 - protein, 15 aa, Artificial sequence
  Synthetic peptide
YFKNHTSPDVDLGDI

SEQ ID NO: 1152 - protein, 15 aa, Artificial sequence
  Synthetic peptide
FKNHTSPDVDLGDIS

SEQ ID NO: 1153 - protein, 15 aa, Artificial sequence
  Synthetic peptide
KNHTSPDVDLGDISG

SEQ ID NO: 1154 - protein, 15 aa, Artificial sequence
  Synthetic peptide
NHTSPDVDLGDISGI

SEQ ID NO: 1155 - protein, 15 aa, Artificial sequence
  Synthetic peptide
HTSPDVDLGDISGIN

SEQ ID NO: 1156 - protein, 15 aa, Artificial sequence
  Synthetic peptide
TSPDVDLGDISGINA

SEQ ID NO: 1157 - protein, 15 aa, Artificial sequence
  Synthetic peptide
SPDVDLGDISGINAS

SEQ ID NO: 1158 - protein, 15 aa, Artificial sequence
  Synthetic peptide
PDVDLGDISGINASV

SEQ ID NO: 1159 - protein, 15 aa, Artificial sequence
  Synthetic peptide
DVDLGDISGINASVV

SEQ ID NO: 1160 - protein, 15 aa, Artificial sequence
  Synthetic peptide
VDLGDISGINASVVN

SEQ ID NO: 1161 - protein, 15 aa, Artificial sequence
  Synthetic peptide
DLGDISGINASVVNI

SEQ ID NO: 1162 - protein, 15 aa, Artificial sequence
  Synthetic peptide
LGDISGINASVVNIQ

SEQ ID NO: 1163 - protein, 15 aa, Artificial sequence
  Synthetic peptide
GDISGINASVVNIQK

SEQ ID NO: 1164 - protein, 15 aa, Artificial sequence
  Synthetic peptide
DISGINASVVNIQKE

SEQ ID NO: 1165 - protein, 15 aa, Artificial sequence
  Synthetic peptide
ISGINASVVNIQKEI

SEQ ID NO: 1166 - protein, 15 aa, Artificial sequence
  Synthetic peptide
SGINASVVNIQKEID

SEQ ID NO: 1167 - protein, 15 aa, Artificial sequence
  Synthetic peptide
GINASVVNIQKEIDR

SEQ ID NO: 1168 - protein, 15 aa, Artificial sequence
  Synthetic peptide
INASVVNIQKEIDRL

SEQ ID NO: 1169 - protein, 15 aa, Artificial sequence
  Synthetic peptide
NASVVNIQKEIDRLN

SEQ ID NO: 1170 - protein, 15 aa, Artificial sequence
  Synthetic peptide
ASVVNIQKEIDRLNE

SEQ ID NO: 1171 - protein, 15 aa, Artificial sequence
  Synthetic peptide
SVVNIQKEIDRLNEV

SEQ ID NO: 1172 - protein, 15 aa, Artificial sequence
  Synthetic peptide
VVNIQKEIDRLNEVA

SEQ ID NO: 1173 - protein, 15 aa, Artificial sequence
  Synthetic peptide
VNIQKEIDRLNEVAK

SEQ ID NO: 1174 - protein, 15 aa, Artificial sequence
  Synthetic peptide
NIQKEIDRLNEVAKN

SEQ ID NO: 1175 - protein, 15 aa, Artificial sequence
  Synthetic peptide
IQKEIDRLNEVAKNL

SEQ ID NO: 1176 - protein, 15 aa, Artificial sequence
  Synthetic peptide
QKEIDRLNEVAKNLN

SEQ ID NO: 1177 - protein, 15 aa, Artificial sequence
  Synthetic peptide
KEIDRLNEVAKNLNE

SEQ ID NO: 1178 - protein, 15 aa, Artificial sequence
  Synthetic peptide
EIDRLNEVAKNLNES

SEQ ID NO: 1179 - protein, 15 aa, Artificial sequence
  Synthetic peptide
IDRLNEVAKNLNESL

SEQ ID NO: 1180 - protein, 15 aa, Artificial sequence
  Synthetic peptide
DRLNEVAKNLNESLI

SEQ ID NO: 1181 - protein, 15 aa, Artificial sequence
  Synthetic peptide
RLNEVAKNLNESLID

SEQ ID NO: 1182 - protein, 15 aa, Artificial sequence
  Synthetic peptide
LNEVAKNLNESLIDL

SEQ ID NO: 1183 - protein, 15 aa, Artificial sequence
  Synthetic peptide
NEVAKNLNESLIDLQ

SEQ ID NO: 1184 - protein, 15 aa, Artificial sequence
  Synthetic peptide
EVAKNLNESLIDLQE

SEQ ID NO: 1185 - protein, 15 aa, Artificial sequence
  Synthetic peptide
VAKNLNESLIDLQEL

SEQ ID NO: 1186 - protein, 15 aa, Artificial sequence
  Synthetic peptide
AKNLNESLIDLQELG

SEQ ID NO: 1187 - protein, 15 aa, Artificial sequence
  Synthetic peptide
KNLNESLIDLQELGK

SEQ ID NO: 1188 - protein, 15 aa, Artificial sequence
  Synthetic peptide
NLNESLIDLQELGKY

SEQ ID NO: 1189 - protein, 15 aa, Artificial sequence
  Synthetic peptide
LNESLIDLQELGKYE

SEQ ID NO: 1190 - protein, 15 aa, Artificial sequence
  Synthetic peptide
NESLIDLQELGKYEQ

SEQ ID NO: 1191 - protein, 15 aa, Artificial sequence
  Synthetic peptide
ESLIDLQELGKYEQY

SEQ ID NO: 1192 - protein, 15 aa, Artificial sequence
  Synthetic peptide
SLIDLQELGKYEQYI

SEQ ID NO: 1193 - protein, 15 aa, Artificial sequence
  Synthetic peptide
LIDLQELGKYEQYIK

SEQ ID NO: 1194 - protein, 15 aa, Artificial sequence
  Synthetic peptide
IDLQELGKYEQYIKW

SEQ ID NO: 1195 - protein, 15 aa, Artificial sequence
  Synthetic peptide
DLQELGKYEQYIKWP

SEQ ID NO: 1196 - protein, 15 aa, Artificial sequence
  Synthetic peptide
LQELGKYEQYIKWPW

SEQ ID NO: 1197 - protein, 15 aa, Artificial sequence
  Synthetic peptide
QELGKYEQYIKWPWY

SEQ ID NO: 1198 - protein, 15 aa, Artificial sequence
  Synthetic peptide
ELGKYEQYIKWPWYV

SEQ ID NO: 1199 - protein, 15 aa, Artificial sequence
  Synthetic peptide
LGKYEQYIKWPWYVW

SEQ ID NO: 1200 - protein, 15 aa, Artificial sequence
  Synthetic peptide
GKYEQYIKWPWYVWL

SEQ ID NO: 1201 - protein, 15 aa, Artificial sequence
  Synthetic peptide
KYEQYIKWPWYVWLG

SEQ ID NO: 1202 - protein, 15 aa, Artificial sequence
  Synthetic peptide
YEQYIKWPWYVWLGF

SEQ ID NO: 1203 - protein, 15 aa, Artificial sequence
  Synthetic peptide
EQYIKWPWYVWLGFI

SEQ ID NO: 1204 - protein, 15 aa, Artificial sequence
  Synthetic peptide
QYIKWPWYVWLGFIA

SEQ ID NO: 1205 - protein, 15 aa, Artificial sequence
  Synthetic peptide
YIKWPWYVWLGFIAG

SEQ ID NO: 1206 - protein, 15 aa, Artificial sequence
  Synthetic peptide
IKWPWYVWLGFIAGL

SEQ ID NO: 1207 - protein, 15 aa, Artificial sequence
  Synthetic peptide
KWPWYVWLGFIAGLI

SEQ ID NO: 1208 - protein, 15 aa, Artificial sequence
  Synthetic peptide
WPWYVWLGFIAGLIA

SEQ ID NO: 1209 - protein, 15 aa, Artificial sequence
  Synthetic peptide
PWYVWLGFIAGLIAI

SEQ ID NO: 1210 - protein, 15 aa, Artificial sequence
  Synthetic peptide
WYVWLGFIAGLIAIV

SEQ ID NO: 1211 - protein, 15 aa, Artificial sequence
  Synthetic peptide
YVWLGFIAGLIAIVM

SEQ ID NO: 1212 - protein, 15 aa, Artificial sequence
  Synthetic peptide
VWLGFIAGLIAIVMV

SEQ ID NO: 1213 - protein, 15 aa, Artificial sequence
  Synthetic peptide
WLGFIAGLIAIVMVT

SEQ ID NO: 1214 - protein, 15 aa, Artificial sequence
  Synthetic peptide
LGFIAGLIAIVMVTI

SEQ ID NO: 1215 - protein, 15 aa, Artificial sequence
  Synthetic peptide
GFIAGLIAIVMVTIL

SEQ ID NO: 1216 - protein, 15 aa, Artificial sequence
  Synthetic peptide
FIAGLIAIVMVTILL

SEQ ID NO: 1217 - protein, 15 aa, Artificial sequence
  Synthetic peptide
IAGLIAIVMVTILLC

SEQ ID NO: 1218 - protein, 15 aa, Artificial sequence
  Synthetic peptide
AGLIAIVMVTILLCC

SEQ ID NO: 1219 - protein, 15 aa, Artificial sequence
  Synthetic peptide
GLIAIVMVTILLCCM

SEQ ID NO: 1220 - protein, 15 aa, Artificial sequence
  Synthetic peptide
LIAIVMVTILLCCMT

SEQ ID NO: 1221 - protein, 15 aa, Artificial sequence
  Synthetic peptide
IAIVMVTILLCCMTS

SEQ ID NO: 1222 - protein, 15 aa, Artificial sequence
  Synthetic peptide
AIVMVTILLCCMTSC

SEQ ID NO: 1223 - protein, 15 aa, Artificial sequence
  Synthetic peptide
IVMVTILLCCMTSCC

SEQ ID NO: 1224 - protein, 15 aa, Artificial sequence
  Synthetic peptide
VMVTILLCCMTSCCS

SEQ ID NO: 1225 - protein, 15 aa, Artificial sequence
  Synthetic peptide
MVTILLCCMTSCCSC

SEQ ID NO: 1226 - protein, 15 aa, Artificial sequence
  Synthetic peptide
VTILLCCMTSCCSCL

SEQ ID NO: 1227 - protein, 15 aa, Artificial sequence
  Synthetic peptide
TILLCCMTSCCSCLK

SEQ ID NO: 1228 - protein, 15 aa, Artificial sequence
  Synthetic peptide
ILLCCMTSCCSCLKG

SEQ ID NO: 1229 - protein, 15 aa, Artificial sequence
  Synthetic peptide
LLCCMTSCCSCLKGA

SEQ ID NO: 1230 - protein, 15 aa, Artificial sequence
  Synthetic peptide
LCCMTSCCSCLKGAC

SEQ ID NO: 1231 - protein, 15 aa, Artificial sequence
  Synthetic peptide
CCMTSCCSCLKGACS

SEQ ID NO: 1232 - protein, 15 aa, Artificial sequence
  Synthetic peptide
CMTSCCSCLKGACSC

SEQ ID NO: 1233 - protein, 15 aa, Artificial sequence
  Synthetic peptide
MTSCCSCLKGACSCG

SEQ ID NO: 1234 - protein, 15 aa, Artificial sequence
  Synthetic peptide
TSCCSCLKGACSCGS

SEQ ID NO: 1235 - protein, 15 aa, Artificial sequence
  Synthetic peptide
SCCKFDEDDSEPVLK

SEQ ID NO: 1236 - protein, 15 aa, Artificial sequence
  Synthetic peptide
CCKFDEDDSEPVLKG

SEQ ID NO: 1237 - protein, 15 aa, Artificial sequence
  Synthetic peptide
CKFDEDDSEPVLKGV

SEQ ID NO: 1238 - protein, 15 aa, Artificial sequence
  Synthetic peptide
KFDEDDSEPVLKGVK

SEQ ID NO: 1239 - protein, 15 aa, Artificial sequence
  Synthetic peptide
FDEDDSEPVLKGVKL

SEQ ID NO: 1240 - protein, 15 aa, Artificial sequence
  Synthetic peptide
DEDDSEPVLKGVKLH

SEQ ID NO: 1241 - protein, 15 aa, Artificial sequence
  Synthetic peptide
EDDSEPVLKGVKLHY

SEQ ID NO: 1242 - protein, 15 aa, Artificial sequence
  Synthetic peptide
DDSEPVLKGVKLHYT

SEQ ID NO: 1243 - protein, 1363 aa, Coronavirus OC43
MFLILLISLPTAFAVIGDLKCTTVSINDIDTGAPSISTDIVDVTNGLGTYYVLDRVYLNTTLLLNGYYPTSGSTYRNMALKGTLLLSRLWFKPPFLSDFINGIFAKVKNTKVIKKGVMYSEFPAITIGSTFVNTSYSVVVQPHTTNLDNKLQGLLEISVCQYTMCEYPHTICHPNLGNRRVELWHWDTGVVSCLYKRNFTYDVNADYLYFHFYQEGGTFYAYFTDTGVVTKFLFNVYLGTVLSHYYVLPLTCNSAMTLEYWVTPLTSKQYLLAFNQDGVIFNAVDCKSDFMSEIKCKTLSIAPSTGVYELNGYTVQPIADVYRRIPNLPDCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNRRFGFTEQSVFKPQPVGVFTHHDVVYAQHCFKAPTNFCPCKLDGSLCVGNGPGIDAGYKNSGIGTCPAGTNYLTCHNAAQCDCLCTPDPITSKSTGPYKCPQTKYLVGIGEHCSGLAIKSDYCGGNPCTCQPQAFLGWSVDSCLQGDRCNIFANFILHDVNSGTTCSTDLQKSNTDIILGVCVNYDLYGITGQGIFVEVNAPYYNSWQNLLYDSNGNLYGFRDYLTNRTFMIRSCYSGRVSAAFHANSSEPALLFRNIKCSYVFNNTLSRQLQPINYFDSYLGCVVNADNSTSSVVQTCDLTVGSGYCVDYSTKRRSRRAITTGYRFTNFEPFTVNSVNDSLEPVGGLYEIQIPSEFTIGNMEEFIQTSSPKVTIDCSAFVCGDYAACKSQLVEYGSFCDNINAILTEVNELLDTTQLQVANSLMNGVTLSTKLKDGVNFNVDDINFSPVLGCLGSACNKVSSRSAIEDLLFSKVKLSDVGFVEAYNNCTGGAEIRDLICVQSYNGIKVLPPLLSVNQISGYTLAATSASLFPPWSAAAGVPFYLNVQYRINGIGVTMDVLSQNQKLIANAFSNALDAIQEGFDATNSALVKIQAVVNANAEALNNLLQQLSNRFGAIGSSLQEILSRLDALEAQAQIDRLINGRLTALNAYVSQQLSDSTLVKFSAAQAMEKVNECVKSQSSRINFCGNGNHIISLVQNAPYGLYFIHFSYVPTKYVTAKVSPGLCIAGDRGIAPKSGYFVNVNNTWMFTGSGYYYPEPITGNNVVVMSTCAVNYTKAPDVMLNISTPNLHDFKEELDQWFKNQTSVAPDLSLDYINVTFLDLQDEMNRLQEAIKVLNQSYINLKDIGTYEYYVKWPWYVWLLIGFAGVAMLVLLFFICCCTGCGTSCFKICGGCCDDYTGHQELVIKTSHDD

SEQ ID NO: 1244 - protein, 13 aa, Artificial sequence
  Synthetic peptide
KPTKRSFIEDLLF

SEQ ID NO: 1245 - protein, 21 aa, Artificial sequence
  Synthetic peptide
VLYENQKQIANQFNKAISQIQ

SEQ ID NO: 1246 - protein, 26 aa, Artificial sequence
  Synthetic peptide
IRAAEIRASANLAATKMSECVLGQSK

SEQ ID NO: 1247 - protein, 48 aa, Artificial sequence
  Synthetic peptide
GYHLMSFPQAAPHGVVFLHVTYVPSQERNFTTAPAICHEGKAYFPREG

SEQ ID NO: 1248 - protein, 16 aa, Artificial sequence
  Synthetic peptide
GTQTHTMIFDNAFNCT

SEQ ID NO: 1249 - protein, 16 aa, Artificial sequence
  Synthetic peptide
SLDVSEKSGNFKHLRE

SEQ ID NO: 1250 - protein, 19 aa, Artificial sequence
  Synthetic peptide
ENGTITDAVDCSQNPLAEL

SEQ ID NO: 1251 - protein, 24 aa, Artificial sequence
  Synthetic peptide
DAVDCSQNPLAELKCSVKSFEIDK

SEQ ID NO: 1252 - protein, 20 aa, Artificial sequence
  Synthetic peptide
SNVPFSPDGKPCTPPALNCY

SEQ ID NO: 1253 - protein, 19 aa, Artificial sequence
  Synthetic peptide
SFGGVSVITPGTNASSEVA

SEQ ID NO: 1254 - protein, 20 aa, Artificial sequence
  Synthetic peptide
QAGCLIGAEHVDTSYECDIP

SEQ ID NO: 1255 - protein, 21 aa, Artificial sequence
  Synthetic peptide
AKTSVDCNMYICGDSTECANL

SEQ ID NO: 1256 - protein, 17 aa, Artificial sequence
  Synthetic peptide
MYICGDSTECANLLLQY

SEQ ID NO: 1257 - protein, 20 aa, Artificial sequence
  Synthetic peptide
LKPTKRSFIEDLLFNKVTLA

SEQ ID NO: 1258 - protein, 17 aa, Artificial sequence
  Synthetic peptide
SCCSCLKGACSCGSCCK

SEQ ID NO: 1259 - protein, 16 aa, Artificial sequence
  Synthetic peptide
CLKGACSCGSCCKFDE

SEQ ID NO: 1260 - protein, 16 aa, Artificial sequence
  Synthetic peptide
TLTSGSDLDRCTTFDD

SEQ ID NO: 1261 - protein, 16 aa, Artificial sequence
  Synthetic peptide
FKNHTSPDVDLGDISG

SEQ ID NO: 1262 - protein, 16 aa, Artificial sequence
  Synthetic peptide
RLNEVAKNLNESLIDL

SEQ ID NO: 1263 - protein, 16 aa, Artificial sequence
  Synthetic peptide
VAKNLNESLIDLQELG

SEQ ID NO: 1264 - protein, 17 aa, Artificial sequence
  Synthetic peptide
TSCCSCLKGACSCGSCC